(19) United States
(12) Patent Application Publication
(10) Pub. No.: US 2019/0241914 A1
(43) Pub. Date: Aug. 8, 2019

(54) Method for Producing L-Methionine or Metabolites Requiring S-Adenosylmethionine for Synthesis

(71) Applicant: AJINOMOTO CO., INC., Tokyo (JP)

(72) Inventors: Benjamin Mijts, San Carlos, CA (US); Christine Roche, Berkeley, CA (US); Peter Kelly, Oakland, CA (US); Sayaka Asari, Kanagawa (JP); Miku Toyazaki, Kanagawa (JP); Keita Fukui, Kanagawa (JP)

(73) Assignee: AJINOMOTO CO., INC., Tokyo (JP)

(21) Appl. No.: 16/392,776

(22) Filed: Apr. 24, 2019

Related U.S. Application Data
(63) Continuation of application No. PCT/JP2017/038798, filed on Oct. 26, 2017.
(60) Provisional application No. 62/413,044, filed on Oct. 26, 2016.
(60) Provisional application No. 62/417,609, filed on Nov. 4, 2016.

(51) Int. Cl. C12P 7/42

(52) U.S. Cl. CPC C12P 7/42

(57) ABSTRACT

A method for producing an objective substance such as vanillin and vanillic acid is provided. An objective substance is produced from a carbon source or a precursor of the objective substance by using a microorganism having an objective substance-producing ability, which microorganism has been modified so that the activity of NCgl2048 protein is reduced.

This application is a Continuation of, and claims priority under 35 U.S.C. § 120 to, International Application No. PCT/JP2017/038798, filed Oct. 26, 2017, and claims priority therethrough under 35 U.S.C. § 119 to U.S. Provisional Patent Application No. 62/413,044, filed Oct. 26, 2016, and U.S. Provisional Patent Application No. 62/417,609, filed Nov. 4, 2016, the entireties of which are incorporated by reference herein. Also, the Sequence Listing filed electronically herewith is hereby incorporated by reference (File name: 2019-04-24T US-552_Seq_List; File size: 156 KB; Date recorded: Apr. 24, 2019).

### BACKGROUND

### General Field

The present invention relates to a method for producing an objective substance such as vanillin and vanillic acid by using a microorganism.

### Brief Description of the Related Art

Vanillin is the major ingredient that provides the smell of vanilla, and is used as an aromatic in foods, drinks, perfumes, and so forth. Vanillin is usually produced by extraction from natural products or by chemical synthesis.

Bioengineering techniques have been tried in methods of producing vanillin, such as by using various microorganisms and raw materials, such as eugenol, isoeugenol, ferulic acid, glucose, vanillic acid, coconut husk, or the like (Kaur B. and Chakraborty D., Biotechnological and molecular approaches for vanillin production: a review. Appl Biochem Biotechnol. 2013 February;169(4):1353-72). In addition, other methods for producing vanillin using bioengineering techniques include producing vanillin as a glycoside (WO2013/022881 and WO2004/111254), producing vanillin from ferulic acid using vanillin synthase (JP2015-535181), producing vanillic acid by fermentation of Escherichia coli and then enzymatically converting vanillic acid into vanillin (U.S. Pat. No. 6,372,461).

The NCgl2048 gene of Corynebacterium glutamicum encodes a protein homologous to both the MetE and MetH proteins, which are encoded by the metE and metH genes, respectively. While the protein encoded by the NCgl2048 gene is annotated as methionine synthase in some databases, the actual function thereof has not been identified.

### SUMMARY

The present invention describes a novel technique for improving production of an objective substance, such as vanillin and vanillic acid, and thereby provides a method for efficiently producing the objective substance.

It is one aspect of the present invention that a microorganism can produce an objective substance such as vanillic acid in a significantly improved manner by modifying the microorganism so that expression of an NCgl2048 gene is attenuated.

It is an aspect of the present invention to provide a method for producing an objective substance, the method comprising the following step: producing the objective substance by using a microorganism having an ability to produce the objective substance, wherein the microorganism has been modified so that the activity of a protein encoded by NCgl2048 gene is reduced as compared with a non-modified strain, and wherein the objective substance is selected from the group consisting of L-methionine, metabolites the biosynthesis of which requires S-adenosylmethionine, and combinations thereof.

It is a further aspect of the present invention to provide the method as described above, wherein said producing comprises cultivating the microorganism in a culture medium containing a carbon source to produce and accumulate the objective substance in the culture medium.

It is a further aspect of the present invention to provide the method as described above, wherein said producing comprises converting a precursor of the objective substance into the objective substance by using the microorganism.

It is a further aspect of the present invention to provide the method as described above, wherein said converting comprises cultivating the microorganism in a culture medium containing the precursor to produce and accumulate the objective substance in the culture medium.

It is a further aspect of the present invention to provide the method as described above, wherein said converting comprises allowing cells of the microorganism to act on the precursor in a reaction mixture to produce and accumulate the objective substance in the reaction mixture.

It is a further aspect of the present invention to provide the method as described above, wherein the cells are cells present in a culture broth of the microorganism, cells collected from the culture broth, cells present in a processed product of the culture broth, cells present in a processed product of the collected cells, or a combination of these.

It is a further aspect of the present invention to provide the method as described above, wherein the precursor is selected from the group consisting of protocatechuic acid, protocatechualdehyde, L-tryptophan, L-histidine, L-phenylalanine, L-tyrosine, L-arginine, L-ornithine, glycine, and combinations thereof.

It is a further aspect of the present invention to provide the method as described above, the method further comprising collecting the objective substance.

It is a further aspect of the present invention to provide the method as described above, wherein the NCgl2048 gene encodes a protein selected from the group consisting of:
(a) a protein comprising the amino acid sequence of SEQ ID NO: 93,
(b) a protein comprising the amino acid sequence of SEQ ID NO: 93 but that includes substitution, deletion, insertion, and/or addition of 1 to 10 amino acid residues, and wherein said protein has a property that a reduction in the activity of the protein in a microorganism results in an increased production of an objective substance, and
(c) a protein comprising an amino acid sequence having an identity of 90% or higher to the amino acid sequence of SEQ ID NO: 93, and wherein said protein has a property that a reduction in the activity of the protein in a microorganism results in an increased production of an objective substance.

It is a further aspect of the present invention to provide the method as described above, wherein the activity of the protein encoded by the NCgl2048 gene is reduced by attenuating the expression of the NCgl2048 gene, or by disrupting the NCgl2048 gene.

It is a further aspect of the present invention to provide the method as described above, wherein the expression of the NCgl2048 gene is attenuated by modifying an expression control sequence of the NCgl2048 gene.

It is a further aspect of the present invention to provide the method as described above, wherein the microorganism is a bacterium belonging to the family Enterobacteriaceae, a coryneform bacterium, or yeast.

It is a further aspect of the present invention to provide the method as described above, wherein the microorganism is a bacterium belonging to the genus Corynebacterium.

It is a further aspect of the present invention to provide the method as described above, wherein the microorganism is Corynebacterium glutamicum.

It is a further aspect of the present invention to provide the method as described above, wherein the microorganism is a bacterium belonging to the genus Escherichia.

It is a further aspect of the present invention to provide the method as described above, wherein the microorganism is Escherichia coli.

It is a further aspect of the present invention to provide the method as described above, wherein the metabolites are selected from the group consisting of vanillin, vanillic acid, melatonin, ergothioneine, mugineic acid, ferulic acid, polyamine, guaiacol, 4-vinylguaiacol, 4-ethylguaiacol, and creatine.

It is a further aspect of the present invention to provide the method as described above, wherein the microorganism has been further modified so that the activity of an enzyme that is involved in the biosynthesis of the objective substance is increased as compared with a non-modified strain.

It is a further aspect of the present invention to provide the method as described above, wherein the enzyme that is involved in the biosynthesis of the objective substance is selected from the group consisting of 3-deoxy-D-arabino-heptulosonic acid 7-phosphate synthase, 3-dehydroquinate synthase, 3-dehydroquinate dehydratase, 3-dehydroshikimate dehydratase, O-methyltransferase, aromatic aldehyde oxidoreductase, and combinations thereof.

It is a further aspect of the present invention to provide the method as described above, wherein the microorganism has been further modified so that the activity of phosphopantetheinyl transferase is increased as compared with a non-modified strain.

It is a further aspect of the present invention to provide the method as described above, wherein the microorganism has been further modified so that the activity of an enzyme that is involved in the by-production of a substance other than the objective substance is reduced as compared with a non-modified strain.

It is a further aspect of the present invention to provide the method as described above, wherein the enzyme that is involved in the by-production of a substance other than the objective substance is selected from the group consisting of vanillate demethylase, protocatechuate 3,4-dioxygenase, alcohol dehydrogenase, shikimate dehydrogenase, and combinations thereof

It is a further aspect of the present invention to provide a method for producing vanillin, the method comprising producing vanillic acid by the method as described above; and converting said vanillic acid to vanillin.

### DETAILED DESCRIPTION OF THE PREFERRED EMBODIMENTS

### <1> Microorganism

The microorganism as described herein is a microorganism that has an ability to produce an objective substance, which microorganism has been modified so that the activity of a NCgl2048 protein, which is a protein encoded by a NCgl2048 gene, is reduced. The ability to produce an objective substance can also be referred to as an “objective substance-producing ability”.

<1-1> Microorganism having Objective Substance-Producing Ability

The phrase “microorganism having an objective substance-producing ability” can refer to a microorganism that is able to produce an objective substance.

The phrase “microorganism having an objective substance-producing ability” can refer to a microorganism that is able to produce an objective substance by fermentation, if the microorganism is used in a fermentation method. That is, the phrase “microorganism having an objective substance-producing ability” can refer to a microorganism that is able to produce an objective substance from a carbon source. Specifically, the phrase “microorganism having an objective substance-producing ability” can refer to a microorganism that is able to, upon being cultured in a culture medium, such as a culture medium containing a carbon source, produce and accumulate the objective substance in the culture medium to such a degree that the objective substance can be collected from the culture medium.

Also, the phrase “microorganism having an objective substance-producing ability” can refer to a microorganism that is able to produce an objective substance by bioconversion, if the microorganism is used in a bioconversion method. That is, the phrase “microorganism having an objective substance-producing ability” can refer to a microorganism that is able to produce an objective substance from a precursor of the objective substance. Specifically, the phrase “microorganism having an objective substance-producing ability” can refer to a microorganism that is able to, upon being cultured in a culture medium containing a precursor of an objective substance, produce and accumulate the objective substance in the culture medium to such a degree that the objective substance can be collected from the culture medium. Also, specifically, the phrase “microorganism having an objective substance-producing ability” can refer to a microorganism that is able to, upon being allowed to act on a precursor of an objective substance in a reaction mixture, produce and accumulate the objective substance in the reaction mixture to such a degree that the objective substance can be collected from the reaction mixture.

The microorganism having an objective substance-producing ability can be able to produce and accumulate the objective substance in the culture medium or reaction mixture in an amount larger than that can be obtained with a non-modified strain. A non-modified strain can also be referred to as a “strain of a non-modified microorganism” or a “non-modified microorganism”. The phrase “strain of a non-modified microorganism” or “non-modified strain” can refer to a control strain that has not been modified so that the activity of NCgl2048 protein is reduced. The microorganism having an objective substance-producing ability can be able to accumulate the objective substance in the culture medium or reaction mixture in an amount of, for example, 0.01 g/L or more, 0.05 g/L or more, or 0.09 g/L or more.

The objective substance can be selected from L-methionine and metabolites the biosynthesis of which requires S-adenosylmethionine (SAM). Examples of metabolites the biosynthesis of which requires SAM can include, for example, vanillin, vanillic acid, melatonin, ergothioneine, mugineic acid, ferulic acid, polyamine, guaiacol, 4-vinylguaiacol, 4-ethylguaiacol, and creatine. Examples of polyamine can include spermidine and spermine. The microorganism may be able to produce only one objective substance, or may be able to produce two or more objective substances. Also, the microorganism may be able to produce an objective substance from one precursor of the objective substance or from two or more precursors of the objective substance.

When the objective substance is a compound that can form a salt, the objective substance may be obtained as a free compound, a salt thereof, or a mixture of these. That is, the term “objective substance” can refer to an objective substance in a free form, a salt thereof, or a mixture thereof, unless otherwise stated. Examples of the salt can include, for example, sulfate salt, hydrochloride salt, carbonate salt, ammonium salt, sodium salt, and potassium salt. As the salt of the objective substance, one kind of salt may be employed, or two or more kinds of salts may be employed in combination.

A microorganism that can be used as a parent strain to construct the microorganism as described herein is not particularly limited. Examples of the microorganism can include bacteria and yeast.

Examples of the bacteria can include bacteria belonging to the family Enterobacteriaceae and coryneform bacteria.

Examples of bacteria belonging to the family Enterobacteriaceae can include bacteria belonging to the genus Escherichia, Enterobacter, Pantoea, Klebsiella, Serratia, Envinia, Photorhabdus, Providencia, Salmonella, Morganella, or the like. Specifically, bacteria classified into the family Enterobacteriaceae according to the taxonomy used in the NCBI (National Center for Biotechnology Information) database (ncbi.nlm.nih.gov/Taxonomy/Browser/wwwtax.cgi?id=91347) can be used.

The Escherichia bacteria are not particularly limited, and examples thereof can include those classified into the genus Escherichia according to the taxonomy known to those skilled in the field of microbiology. Examples of the Escherichia bacteria can include, for example, those described in the work of Neidhardt et al. (Backmann B. J., 1996, Derivations and Genotypes of some mutant derivatives of Escherichia coli K-12, pp. 2460-2488, Table 1, In F. D. Neidhardt (ed.), Escherichia coli and Salmonella Cellular and Molecular Biology/Second Edition, American Society for Microbiology Press, Washington, D.C.). Examples of the Escherichia bacteria can include, for example, Escherichia coli. Specific examples of Escherichia coli can include, for example, Escherichia coli K-12 strains such as W3110 strain (ATCC 27325) and MG1655 strain (ATCC 47076); Escherichia coli K5 strain (ATCC 23506); Escherichia coli B strains such as BL21 (DE3) strain; and derivative strains thereof.

The Enterobacter bacteria are not particularly limited, and examples can include those classified into the genus Enterobacter according to the taxonomy known to those skilled in the field of microbiology. Examples the Enterobacter bacterium can include, for example, Enterobacter agglomerans and Enterobacter aerogenes. Specific examples of Enterobacter agglomerans can include, for example, the Enterobacter agglomerans ATCC 12287 strain. Specific examples of Enterobacter aerogenes can include, for example, the Enterobacter aerogenes ATCC 13048 strain, NBRC 12010 strain (Biotechnol. Bioeng., 2007, March 27;98(2):340-348), and AJ110637 strain (FERM BP-10955). Examples the Enterobacter bacteria can also include, for example, the strains described in European Patent Application Laid-open (EP-A) No. 0952221. In addition, Enterobacter agglomerans can also include some strains classified as Pantoea agglomerans.

The Pantoea bacteria are not particularly limited, and examples can include those classified into the genus Pantoea according to the taxonomy known to those skilled in the field of microbiology. Examples the Pantoea bacteria can include, for example, Pantoea ananatis, Pantoea stewartii, Pantoea agglomerans, and Pantoea citrea. Specific examples of Pantoea ananatis can include, for example, the Pantoea ananatis LMG20103 strain, AJ13355 strain (FERM BP-6614), AJ13356 strain (FERM BP-6615), AJ13601 strain (FERM BP-7207), SC17 strain (FERM BP-11091), SC17(0) strain (VKPM B-9246), and SC17sucA strain (FERM BP-8646). Some of Enterobacter bacteria and Envinia bacteria were reclassified into the genus Pantoea (Int. J. Syst. Bacteriol., 39, 337-345 (1989); Int. J. Syst. Bacteriol., 43, 162-173 (1993)). For example, some strains of Enterobacter agglomerans were recently reclassified into Pantoea agglomerans, Pantoea ananatis, Pantoea stewartii, or the like on the basis of nucleotide sequence analysis of 16S rRNA etc. (Int. J. Syst. Bacteriol., 39, 337-345 (1989)). The Pantoea bacteria can include those reclassified into the genus Pantoea as described above.

Examples of the Envinia bacteria can include Envinia amylovora and Envinia carotovora. Examples of the Klebsiella bacteria can include Klebsiella planticola.

Examples of coryneform bacteria can include bacteria belonging to the genus Corynebacterium, Brevibacterium, Microbacterium, or the like.

Specific examples of such coryneform bacteria can include the following species:

Corynebacterium acetoacidophilum

Corynebacterium acetoglutamicum

Corynebacterium alkanolyticum

Corynebacterium callunae

Corynebacterium crenatum

Corynebacterium glutamicum

Corynebacterium lilium

Corynebacterium melassecola

Corynebacterium thermoaminogenes (Corynebacterium efficiens)

Corynebacterium herculis

Brevibacterium divaricatum (Corynebacterium glutamicum)

Brevibacterium flavum (Corynebacterium glutamicum)

Brevibacterium immariophilum

Brevibacterium lactofermentum (Corynebacterium glutamicum)

Brevibacterium roseum

Brevibacterium saccharolyticum

Brevibacterium thiogenitalis

Corynebacterium ammoniagenes (Corynebacterium stationis)

Brevibacterium album

Brevibacterium cerinum

Microbacterium ammoniaphilum

Specific examples of the coryneform bacteria can include the following strains:

Corynebacterium acetoacidophilum ATCC 13870

Corynebacterium acetoglutamicum ATCC 15806

Corynebacterium alkanolyticum ATCC 21511

Corynebacterium callunae ATCC 15991

Corynebacterium crenatum AS1.542

Corynebacterium glutamicum ATCC 13020, ATCC 13032, ATCC 13060, ATCC 13869, FERM BP-734

Corynebacterium lilium ATCC 15990

Corynebacterium melassecola ATCC 17965

Corynebacterium efficiens (Corynebacterium thermoaminogenes) AJ12340 (FERM BP-1539)

Corynebacterium herculis ATCC 13868

Brevibacterium divaricatum (Corynebacterium glutamicum) ATCC 14020

Brevibacterium flavum (Corynebacterium glutamicum) ATCC 13826, ATCC 14067, AJ12418 (FERM BP-2205)

Brevibacterium immariophilum ATCC 14068

Brevibacterium lactofermentum (Corynebacterium glutamicum) ATCC 13869

Brevibacterium roseum ATCC 13825

Brevibacterium saccharolyticum ATCC 14066

Brevibacterium thiogenitalis ATCC 19240

Corynebacterium ammoniagenes (Corynebacterium stationis) ATCC 6871, ATCC 6872

Brevibacterium album ATCC 15111

Brevibacterium cerinum ATCC 15112

Microbacterium ammoniaphilum ATCC 15354

The coryneform bacteria can include bacteria that had previously been classified into the genus Brevibacterium, but are now united into the genus Corynebacterium (Int. J. Syst. Bacteriol., 41, 255 (1991)). Moreover, Corynebacterium stationis can include bacteria that had previously been classified as Corynebacterium ammoniagenes, but are now re-classified into Corynebacterium stationis on the basis of nucleotide sequence analysis of 16S rRNA etc. (Int. J. Syst. Evol. Microbiol., 60, 874-879 (2010)).

The yeast may be a budding or fission yeast. The yeast may be a haploid, diploid, or more polyploid yeast. Examples of the yeast can include yeast belonging to the genus Saccharomyces such as Saccharomyces cerevisiae; the genus Pichia, which can also be referred to as the genus Wickerhamomyces, such as Pichia ciferrii, Pichia sydowiorum, and Pichia pastoris; the genus Candida such as Candida utilis; the genus Hansenula such as Hansenula polymorpha; and the genus Schizosaccharomyces such as Schizosaccharomyces pombe

These strains are available from, for example, the American Type Culture Collection (Address: P.O. Box 1549, Manassas, Va. 20108, United States of America; or atcc.org). That is, registration numbers are given to the respective strains, and the strains can be ordered using these registration numbers (refer to atcc.org). The registration numbers of the strains are listed in the catalogue of the American Type Culture Collection. These strains can also be obtained from, for example, the depositories at which the strains were deposited.

The microorganism may inherently have an objective substance-producing ability, or may have been modified so that it has an objective substance-producing ability. The microorganism having an objective substance-producing ability can be obtained by imparting an objective substance-producing ability to such a microorganism as described above, or enhancing an objective substance-producing ability of such a microorganism as mentioned above.

Hereafter, specific examples of the methods for imparting or enhancing an objective substance-producing ability will be explained. Such modifications as exemplified below for imparting or enhancing an objective substance-producing ability may be employed independently, or in an appropriate combination.

An objective substance can be generated by the action of an enzyme that is involved in the biosynthesis of the objective substance. Such an enzyme can also be referred to as an “objective substance biosynthesis enzyme”. Therefore, the microorganism may have an objective substance biosynthesis enzyme. In other words, the microorganism may have a gene encoding an objective substance biosynthesis enzyme. Such a gene can also be referred to as an “objective substance biosynthesis gene”. The microorganism may inherently have an objective substance biosynthesis gene, or may have been introduced with an objective substance biosynthesis gene. The methods for introducing a gene will be explained herein.

Also, an objective substance-producing ability of a microorganism can be improved by increasing the activity of an objective substance biosynthesis enzyme. That is, examples of the method for imparting or enhancing an objective substance-producing ability can include a method of increasing the activity of an objective substance biosynthesis enzyme. That is, the microorganism can be modified so that the activity of an objective substance biosynthesis enzyme is increased. The activity of one objective substance biosynthesis enzyme may be increased, or the activities of two or more objective substance biosynthesis enzymes may be increased. The method for increasing the activity of a protein, such as an enzyme etc., will be described herein. The activity of a protein, such as an enzyme etc., can be increased by, for example, increasing the expression of a gene encoding the protein.

An objective substance can be generated from, for example, a carbon source and/or a precursor of the objective substance. Hence, examples of the objective substance biosynthesis enzyme can include, for example, enzymes that catalyze the conversion of the carbon source and/or the precursor into the objective substance. For example, 3-dehydroshikimic acid can be produced via a part of shikimate pathway, which may include steps catalyzed by 3-deoxy-D-arabino-heptulosonic acid 7-phosphate synthase (DAHP synthase), 3-dehydroquinate synthase, and 3-dehydroquinate dehydratase; 3-dehydroshikimic acid can be converted to protocatechuic acid by the action of 3-dehydroshikimate dehydratase (DHSD); protocatechuic acid can be converted to vanillic acid or protocatechualdehyde by the action of O-methyltransferase (OMT) or aromatic aldehyde oxidoreductase, such as aromatic carboxylic acid reductase; ACAR, respectively; and vanillic acid or protocatechualdehyde can be converted to vanillin by the action of ACAR or OMT, respectively. That is, specific examples of the objective substance biosynthesis enzyme can include, for example, DAHP synthase, 3-dehydroquinate synthase, 3-dehydroquinate dehydratase, DHSD, OMT, and ACAR.

The term “3-deoxy-D-arabino-heptulosonic acid 7-phosphate synthase (DAHP synthase)” can refer to a protein that has the activity of catalyzing the reaction of converting D-erythrose 4-phosphate and phosphoenolpyruvic acid into 3-deoxy-D-arabino-heptulosonate 7-phosphate (DAHP) and phosphate (EC 2.5.1.54). A gene encoding a DAHP synthase can also be referred to as a “DAHP synthase gene”. Examples of a DAHP synthase can include the AroF, AroG, and AroH proteins, which are encoded by the aroF, aroG, and aroH genes, respectively. Among these, AroG may function as the major DAHP synthase. Examples of a DAHP synthase such as the AroF, AroG, and AroH proteins can include those native to various organisms such as Enterobacteriaceae bacteria and coryneform bacteria. Specific examples of a DAHP synthase can include the AroF, AroG, and AroH proteins native to E. coli. The nucleotide sequence of the aroG gene native to the E. coli K-12 MG1655 strain is shown as SEQ ID NO: 1, and the amino acid sequence of the AroG protein encoded by this gene is shown as SEQ ID NO: 2.

The DAHP synthase activity can be measured by, for example, incubating the enzyme with substrates, such as D-erythrose 4-phosphate and phosphoenolpyruvic acid, and measuring the enzyme- and substrate-dependent generation of DAHP.

The term “3-dehydroquinate synthase” can refer to a protein that has the activity of catalyzing the reaction of dephosphorylating DAHP to generate 3-dehydroquinic acid (EC 4.2.3.4). A gene encoding a 3-dehydroquinate synthase can also be referred to as a “3-dehydroquinate synthase gene”. Examples of a 3-dehydroquinate synthase can include the AroB protein, which is encoded by the aroB gene. Examples of a 3-dehydroquinate synthase such as the AroB protein can include those native to various organisms such as Enterobacteriaceae bacteria and coryneform bacteria. Specific examples of a 3-dehydroquinate synthase can include the AroB native to E. coli. The nucleotide sequence of the aroB gene native to the E. coli K-12 MG1655 strain is shown as SEQ ID NO: 3, and the amino acid sequence of the AroB protein encoded by this gene is shown as SEQ ID NO: 4.

The 3-dehydroquinate synthase activity can be measured by, for example, incubating the enzyme with a substrate, such as DAHP, and measuring the enzyme- and substrate-dependent generation of 3-dehydroquinic acid.

The term “3-dehydroquinate dehydratase” can refer to a protein that has the activity of catalyzing the reaction of dehydrating 3-dehydroquinic acid to generate 3-dehydroshikimic acid (EC 4.2.1.10). A gene encoding a 3-dehydroquinate dehydratase can also be referred to as a “3-dehydroquinate dehydratase gene”. Examples of a 3-dehydroquinate dehydratase can include the AroD protein, which is encoded by the aroD gene. Examples of a 3-dehydroquinate dehydratase such as the AroD protein can include those native to various organisms such as Enterobacteriaceae bacteria and coryneform bacteria. Specific examples of a 3-dehydroquinate dehydratase can include the AroD protein native to E. coli. The nucleotide sequence of the aroD gene native to the E. coli K-12 MG1655 strain is shown as SEQ ID NO: 5, and the amino acid sequence of the AroD protein encoded by this gene is shown as SEQ ID NO: 6.

The 3-dehydroquinate dehydratase activity can be measured by, for example, incubating the enzyme with a substrate, such as 3-dehydroquinic acid, and measuring the enzyme- and substrate-dependent generation of 3-dehydroshikimic acid.

The term “3-dehydroshikimate dehydratase (DHSD)” can refer to a protein that has the activity of catalyzing the reaction of dehydrating 3-dehydroshikimic acid to generate protocatechuic acid (EC 4.2.1.118). A gene encoding a DHSD can also be referred to as a “DHSD gene”. Examples of a DHSD can include the AsbF protein, which is encoded by the asbF gene. Examples of a DHSD such as the AsbF protein can include those native to various organisms such as Bacillus thuringiensis, Neurospora crassa, and Podospora pauciseta. The nucleotide sequence of the asbF gene native to the Bacillus thuringiensis BMB171 strain is shown as SEQ ID NO: 7, and the amino acid sequence of the AsbF protein encoded by this gene is shown as SEQ ID NO: 8.

The DHSD activity can be measured by, for example, incubating the enzyme with a substrate, such as 3-dehydroshikimic acid, and measuring the enzyme- and substrate-dependent generation of protocatechuic acid.

The expression of a gene encoding an enzyme of the shikimate pathway, such as a DAHP synthase, 3-dehydroquinate synthase, and 3-dehydroquinate dehydratase, is repressed by the tyrosine repressor TyrR, which is encoded by the tyrR gene. Therefore, the activity of an enzyme of the shikimate pathway can also be increased by reducing the activity of the tyrosine repressor TyrR. The nucleotide sequence of the tyrR gene native to the E. coli K-12 MG1655 strain is shown as SEQ ID NO: 9, and the amino acid sequence of the TyrR protein encoded by this gene is shown as SEQ ID NO: 10.

The term “O-methyltransferase (OMT)” can refer to a protein that has the activity of catalyzing the reaction of methylating hydroxyl group of a substance in the presence of a methyl group donor (EC 2.1.1.68 etc.). This activity can also be referred to as an “OMT activity”. A gene encoding OMT can also be referred to as an “OMT gene”. OMT can have a required substrate specificity depending on the specific biosynthesis pathway via which an objective substance is produced in the method as described herein. For example, when an objective substance is produced via the conversion of protocatechuic acid into vanillic acid, OMT that is specific for at least protocatechuic acid can be used. Also, for example, when an objective substance is produced via the conversion of protocatechualdehyde into vanillin, OMT that is specific for at least protocatechualdehyde can be used. That is, specifically, the term “O-methyltransferase (OMT)” can refer to a protein that has the activity of catalyzing the reaction of methylating protocatechuic acid and/or protocatechualdehyde in the presence of a methyl group donor to generate vanillic acid and/or vanillin, that is, methylation of hydroxyl group at the meta-position. OMT may be specific for both protocatechuic acid and protocatechualdehyde as the substrate, but is not necessarily limited thereto. Examples of the methyl group donor can include S-adenosylmethionine (SAM). Examples of OMT can include OMTs native to various organisms, such as OMT native to Homo sapiens (Hs) (GenBank Accession No. NP_000745 and NP_009294), OMT native to Arabidopsis thaliana (GenBank Accession Nos. NP_200227 and NP_009294), OMT native to Fragaria x ananassa (GenBank Accession No. AAF28353), and other various OMTs native to mammals, plants, and microorganisms exemplified in WO2013/022881A1. Four kinds of transcript variants and two kinds of OMT isoforms are known for the OMT gene native to Homo sapiens. The nucleotide sequences of these four transcript variants (transcript variant 1-4, GenBank Accession No. NM_000754.3, NM_001135161.1, NM_001135162.1, and NM_007310.2) are shown as SEQ ID NOS: 11 to 14, the amino acid sequence of the longer OMT isoform (MB-COMT, GenBank Accession No. NP_000745.1) is shown as SEQ ID NO: 15, and the amino acid sequence of the shorter OMT isoform (S-COMT, GenBank Accession No. NP_009294.1) is shown as SEQ ID NO: 16. SEQ ID NO: 16 corresponds to SEQ ID NO: 15 of which the N-terminal 50 amino acid residues are truncated.

OMT may also catalyze the reaction of methylating protocatechuic acid and/or protocatechualdehyde to generate isovanillic acid and/or isovanillin, that is, methylation of hydroxyl group at the para-position, as a side reaction. OMT may selectively catalyze the methylation of a hydroxyl group at the meta-position. The expression “selectively catalyzing the methylation of hydroxyl group at the meta-position” can mean that OMT selectively generates vanillic acid from protocatechuic acid and/or that OMT selectively generates vanillin from protocatechualdehyde. The expression “selectively generating vanillic acid from protocatechuic acid” can mean that OMT generates vanillic acid in an amount of, for example, 3 times or more, 5 times or more, 10 times or more, 15 times or more, 20 times or more, 25 times or more, or 30 times or more of that of isovanillic acid in terms of molar ratio, when OMT is allowed to act on protocatechuic acid. Also, the expression “selectively generating vanillic acid from protocatechualdehyde” can mean that OMT generates vanillin in an amount of, for example, 3 times or more, 5 times or more, 10 times or more, 15 times or more, 20 times or more, 25 times or more, or 30 times or more of that of isovanillin in terms of molar ratio, when OMT is allowed to act on protocatechualdehyde. Examples of OMT that selectively catalyzes the methylation of hydroxyl group at the meta-position can include an OMT having a “specific mutation”, which is described herein.

OMT having a “specific mutation” can also be referred to as a “mutant OMT”. A gene encoding a mutant OMT can also be referred to as a “mutant OMT gene”.

OMT not having a “specific mutation” can also be referred to as a “wild-type OMT”. A gene encoding a wild-type OMT can also be referred to as a “wild-type OMT gene”. The term “wild-type” referred to herein is used for convenience to distinguish the “wild-type” OMT from the “mutant” OMT, and the “wild-type” OMT is not limited to those obtained as natural substances, and can include any OMT not having the “specific mutation”. Examples of the wild-type OMT can include, for example, OMTs exemplified above. In addition, all conservative variants of OMTs exemplified above should be included in wild-type OMTs, provided that such conservative variants do not have the “specific mutation”.

Examples of a “specific mutation” can include the mutations contained in the mutant OMTs described in WO2013/022881A1. That is, examples of a “specific mutation” can include a mutation in which the leucine residue at position 198 of the wild-type OMT (L198) is replaced with an amino acid residue having a hydrophobic index (hydropathy index) lower than that of a leucine residue, and a mutation in which the glutamate residue at position 199 of the wild-type OMT (E199) is replaced with an amino acid residue having either a neutral or positive side-chain charge at pH 7.4. The mutant OMT may have either one or both of these mutations.

Examples of the “amino acid residue having a hydrophobic index (hydropathy index) lower than that of leucine residue” can include Ala, Arg, Asn, Asp, Cys, Glu, Gln, Gly, His, Lys, Met, Phe, Pro, Ser, Thr, Trp, and Tyr. As the “amino acid residue showing a hydrophobic index (hydropathy index) lower than that of leucine residue”, especially, an amino acid residue selected from Ala, Arg, Asn, Asp, Glu, Gln, Gly, His, Lys, Met, Pro, Ser, Thr, Trp, and Tyr is a particular example, and Tyr is a more particular example.

The “amino acid residue having either a neutral or positive side-chain charge at pH 7.4” can include Ala, Arg, Asn, Cys, Gln, Gly, His, Ile, Leu, Lys, Met, Phe, Pro, Ser, Thr, Trp, Tyr, and Val. As the “amino acid residue having either a neutral or positive side-chain charge at pH 7.4”, Ala and Gln are particular examples.

The terms “L198” and “E199” in an arbitrary wild-type OMT can refer to “an amino acid residue corresponding to the leucine residue at position 198 of the amino acid sequence shown as SEQ ID NO: 16” and “an amino acid residue corresponding to the glutamate residue at position 199 of the amino acid sequence shown as SEQ ID NO: 16”, respectively. The positions of these amino acid residues represent relative positions, and their absolute positions may shift due to deletion, insertion, addition, and so forth of amino acid residue(s). For example, if one amino acid residue is deleted or inserted at a position on the N-terminus side of position X in the amino acid sequence shown as SEQ ID NO: 16, the amino acid residue originally at position X is relocated at position X−1 or X+1, however, it is still regarded as the “amino acid residue corresponding to the amino acid residue at position X of the amino acid sequence shown as SEQ ID NO: 16”. Furthermore, although “L198” and “E199” are usually leucine residue and glutamate residue, respectively, they may not be leucine residue and glutamate residue, respectively. That is, when “L198” and “E199” are not leucine residue and glutamate residue, respectively, the “specific mutation” can include a mutation in which those amino acid residues each are replaced with any of the aforementioned amino acid residues.

In the amino acid sequence of an arbitrary OMT, which amino acid residue is the amino acid residue corresponding to “L198” or “E199” can be determined by aligning the amino acid sequence of the arbitrary OMT and the amino acid sequence of SEQ ID NO: 16. The alignment can be performed by, for example, using known gene analysis software. Specific examples of such software can include DNASIS produced by Hitachi Solutions, GENETYX produced by Genetyx, and so forth (Elizabeth C. Tyler et al., Computers and Biomedical Research, 24 (1) 72-96, 1991; Barton G J et al., Journal of Molecular Biology, 198 (2), 327-37, 1987).

A mutant OMT gene can be obtained by, for example, modifying a wild-type OMT gene so that OMT encoded thereby has the “specific mutation”. The wild-type OMT gene to be modified can be obtained by, for example, cloning from an organism having the wild-type OMT gene, or chemical synthesis. Furthermore, a mutant OMT gene can also be obtained without using a wild-type OMT gene. For example, a mutant OMT gene may be directly obtained by chemical synthesis. The obtained mutant OMT gene may be used as it is, or may be further modified before use.

Genes can be modified using a known method. For example, an objective mutation can be introduced into a target site of DNA by the site-specific mutagenesis method. Examples of the site-specific mutagenesis method can include a method using PCR (Higuchi, R., 61, in PCR Technology, Erlich, H. A. Eds., Stockton Press (1989); Carter P., Meth. In Enzymol., 154, 382 (1987)), and a method of using a phage (Kramer, W. and Frits, H. J., Meth. in Enzymol., 154, 350 (1987); Kunkel, T. A. et al., Meth. in Enzymol., 154, 367 (1987)).

The OMT activity can be measured by, for example, incubating the enzyme with a substrate, such as protocatechuic acid or protocatechualdehyde, in the presence of SAM, and measuring the enzyme- and substrate-dependent generation of the corresponding product, such as vanillic acid or vanillin (WO2013/022881A1). Furthermore, by measuring the generation of the corresponding by-product, such as isovanillic acid or isovanillin, under the same conditions, and comparing the generation of the by-product with the generation of the product, it can be determined whether OMT selectively generates the product.

The term “aromatic aldehyde oxidoreductase (aromatic carboxylic acid reductase; ACAR)” can refer to a protein that has an activity of catalyzing the reaction of reducing vanillic acid and/or protocatechuic acid in the presence of an electron donor and ATP to generate vanillin and/or protocatechualdehyde (EC 1.2.99.6 etc.). This activity can also be referred to as “ACAR activity”. A gene encoding ACAR can also be referred to as an “ACAR gene”. ACAR may generally use both vanillic acid and protocatechuic acid as the substrate, but is not necessarily limited thereto. That is, ACAR can have a required substrate specificity depending on the specific biosynthesis pathway via which an objective substance is produced in the method as described herein. For example, when an objective substance is produced via the conversion of vanillic acid into vanillin, ACAR that is specific for at least vanillic acid can be used. Also, for example, when an objective substance is produced via the conversion of protocatechuic acid into protocatechualdehyde, ACAR that is specific for at least protocatechuic acid can be used. Examples of the electron donor can include NADH and NADPH. Examples of ACAR can include ACARs native to various organisms such as Nocardia sp. strain NRRL 5646, Actinomyces sp., Clostridium thermoaceticum, Aspergillus niger, Corynespora melonis, Coriolus sp., and Neurospora sp. (J. Biol. Chem., 2007, Vol. 282, No. 1, pp. 478-485). The Nocardia sp. strain NRRL 5646 has been classified into Nocardia iowensis. Examples of ACAR further can include ACARs native to other Nocardia bacteria such as Nocardia brasiliensis and Nocardia vulneris. The nucleotide sequence of the ACAR gene native to Nocardia brasiliensis ATCC 700358 is shown as SEQ ID NO: 17, and the amino acid sequence of ACAR encoded by this gene is shown as SEQ ID NO: 18. The nucleotide sequence of an example of variant ACAR gene native to Nocardia brasiliensis ATCC 700358 is shown as SEQ ID NO: 19, and the amino acid sequence of ACAR encoded by this gene is shown as SEQ ID NO: 20.

The ACAR activity can be measured by, for example, incubating the enzyme with a substrate, such as vanillic acid or protocatechuic acid, in the presence of ATP and NADPH, and measuring the enzyme- and substrate-dependent oxidation of NADPH (modification of the method described in J. Biol. Chem., 2007, Vol. 282, No. 1, pp. 478-485).

ACAR can be made into an active enzyme by phosphopantetheinylation (J. Biol. Chem., 2007, Vol. 282, No. 1, pp. 478-485). Therefore, ACAR activity can also be increased by increasing the activity of an enzyme that catalyzes phosphopantetheinylation of a protein, which can also be referred to as a “phosphopantetheinylation enzyme”. That is, examples of the method for imparting or enhancing an objective substance-producing ability can include a method of increasing the activity of a phosphopantetheinylation enzyme. That is, the microorganism can be modified so that the activity of a phosphopantetheinylation enzyme is increased. Examples of the phosphopantetheinylation enzyme can include phosphopantetheinyl transferase (PPT).

The term “phosphopantetheinyl transferase (PPT)” can refer to a protein that has an activity of catalyzing the reaction of phosphopantetheinylating ACAR in the presence of a phosphopantetheinyl group donor. This activity can also be referred to as “PPT activity”. A gene encoding PPT can also be referred to as a “PPT gene”. Examples of the phosphopantetheinyl group donor can include coenzyme A (CoA). Examples of PPT can include the EntD protein, which is encoded by the entD gene. Examples of PPT such as the EntD protein can include those native to various organisms. Specific examples of PPT can include the EntD protein native to E. coli. The nucleotide sequence of the entD gene native to the E. coli K-12 MG1655 strain is shown as SEQ ID NO: 21, and the amino acid sequence of the EntD protein encoded by this gene is shown as SEQ ID NO: 22. Specific examples of PPT can also include PPT native to Nocardia brasiliensis, PPT native to Nocardia farcinica IFM10152 (J. Biol. Chem., 2007, Vol. 282, No. 1, pp. 478-485), and PPT native to Corynebacterium glutamicum (App. Env. Microbiol. 2009, Vol.75, No.9, pp. 2765-2774). The nucleotide sequence of the PPT gene native to the C. glutamicum ATCC 13032 strain is shown as SEQ ID NO: 23, and the amino acid sequence of PPT encoded by this gene is shown as SEQ ID NO: 24.

The PPT activity can be measured on the basis of, for example, enhancement of the ACAR activity observed when the enzyme is incubated with ACAR in the presence of CoA (J. Biol. Chem., 2007, Vol. 282, No. 1, pp. 478-485).

Melatonin can be produced from L-tryptophan. That is, examples of the objective substance biosynthesis enzyme can also include, for example, L-tryptophan biosynthesis enzymes and enzymes that catalyze the conversion of L-tryptophan into melatonin. Examples of the L-tryptophan biosynthesis enzymes can include common biosynthesis enzymes of aromatic amino acids, such as 3-deoxy-D-arabinoheptulosonate-7-phosphate synthase (aroF, aroG, aroH), 3-dehydroquinate synthase (aroB), 3-dehydroquinate dehydratase (aroD), shikimate dehydrogenase (aroF), shikimate kinase (aroK, aroL), 5-enolpyruvylshikimate-3-phosphate synthase (aroA), and chorismate synthase (aroC); as well as anthranilate synthase (trpED), and tryptophan synthase (trpAB). Shown in the parentheses after the names of the enzymes are examples of the names of the genes encoding the enzymes (the same shall apply to the same occasions hereafter). L-tryptophan can be converted successively to ydroxytryptophan, serotonin, N-acetylserotonin, and melatonin by the action of tryptophan 5-hydroxylase (EC 1.14.16.4), 5-hydroxytryptophan decarboxylase (EC 4.1.1.28), aralkylamine N-acetyltransferase (AANAT; EC 2.3.1.87), and acetylserotonin O-methyltransferase (EC 2.1.1.4). That is, examples of enzymes that catalyze the conversion of L-tryptophan into melatonin can include these enzymes. Notably, acetylserotonin O-methyltransferase is an example of an OMT that catalyzes the reaction of methylating N-acetylserotonin to generate melatonin, using SAM as the methyl donor.

Ergothioneine can be produced from L-histidine. That is, examples of the objective substance biosynthesis enzyme can also include, for example, L-histidine biosynthesis enzymes and enzymes that catalyze the conversion of L-histidine into ergothioneine. Examples of the L-histidine biosynthesis enzymes can include ATP phosphoribosyltransferase (hisG), phosphoribosyl AMP cyclohydrolase (hisI), phosphoribosyl-ATP pyrophosphohydrolase (hisI), phosphoribosylformimino-5-aminoimidazole carboxamide ribotide isomerase (hisA), amidotransferase (hisH), histidinol phosphate aminotransferase (hisC), histidinol phosphatase (hisB), and histidinol dehydrogenase (hisD). L-histidine can be converted successively to hercynine, hercynyl-gamma-L-glutamyl-L-cysteine sulfoxide, hercynyl-L-cysteine sulfoxide, and ergothioneine by the action of the EgtB, EgtC, EgtD, and EgtE proteins, which are encoded by the egtB, egtC, egtD, and egtE genes, respectively. Hercynine can also be converted to hercynyl-L-cysteine sulfoxide by the action of the Egtl protein, which is encoded by the egtI gene. That is, examples of the enzymes that catalyze the conversion of L-histidine into ergothioneine can include these enzymes. Notably, EgtD is an S-adenosyl-1-methionine (SAM)-dependent histidine N,N,N-methyltransferase that catalyzes the reaction of methylating histidine to generate hercynine, using SAM as the methyl donor.

Guaiacol can be produced from vanillic acid. Hence, the aforementioned descriptions concerning objective substance biosynthesis enzymes for vanillic acid can be applied mutatis mutandis to objective substance biosynthesis enzymes for guaiacol. Vanillic acid can be converted to guaiacol by the action of vanillic acid decarboxylase (VDC). That is, examples of the objective substance biosynthesis enzyme can also include VDC.

Ferulic acid, 4-vinylguaiacol, and 4-ethylguaiacol can be produced from L-phenylalanine or L-tyrosine. That is, examples of the objective substance biosynthesis enzyme can also include, for example, L-phenylalanine biosynthesis enzymes, L-tyrosine biosynthesis enzymes, and enzymes that catalyze the conversion of L-phenylalanine or L-tyrosine into ferulic acid, 4-vinylguaiacol, or 4-ethylguaiacol. Examples of the L-phenylalanine biosynthesis enzymes can include the common biosynthesis enzymes of aromatic amino acids exemplified above, as well as chorismate mutase (pheA), prephenate dehydratase (pheA), and tyrosine amino transferase (tyrB). Chorismate mutase and prephenate dehydratase may be encoded by the pheA gene as a bifunctional enzyme. Examples of the L-tyrosine biosynthesis enzymes can include the common biosynthesis enzymes of aromatic amino acids exemplified above, as well as chorismate mutase (tyrA), prephenate dehydrogenase (tyrA), and tyrosine amino transferase (tyrB). Chorismate mutase and prephenate dehydrogenase may be encoded by the tyrA gene as a bifunctional enzyme. L-phenylalanine can be converted to cinnamic acid by the action of phenylalanine ammonia lyase (PAL; EC 4.3.1.24), and then to p-coumaric acid by the action of cinnamic acid 4-hydroxylase (C4H; EC 1.14.13.11). Also, L-tyrosine can be converted to p-coumaric acid by the action of tyrosine ammonia lyase (TAL; EC 4.3.1.23). p-Coumaric acid can be converted successively to caffeic acid, ferulic acid, 4-vinylguaiacol, and 4-ethylguaiacol by the action of hydroxycinnamic acid 3-hydroxylase (C3H), O-methyltransferase (OMT), ferulic acid decarboxylase (FDC), and vinylphenol reductase (VPR), respectively. That is, examples of enzymes that catalyze the conversion of L-phenylalanine or L-tyrosine into ferulic acid, 4-vinylguaiacol, or 4-ethylguaiacol can include these enzymes. For producing ferulic acid, 4-vinylguaiacol, or 4-ethylguaiacol, OMT that uses at least caffeic acid can be used.

Polyamines can be produced from L-arginine or L-ornithine. That is, examples of the objective substance biosynthesis enzyme can also include, for example, L-arginine biosynthesis enzymes, L-ornithine biosynthesis enzymes, and enzymes that catalyze the conversion of L-arginine or L-ornithine into a polyamine. Examples of the L-ornithine biosynthesis enzymes can include N-acetylglutamate synthase (argA), N-acetylglutamate kinase (argB), N-acetylglutamyl phosphate reductase (argC), acetylornithine transaminase (argD), and acetylornithine deacetylase (argE). Examples of the L-arginine biosynthesis enzymes can include the L-ornithine biosynthesis enzymes exemplified above, as well as carbamoyl phosphate synthetase (carAB), ornithine carbamoyl transferase (argF, argI), argininosuccinate synthetase (argG), argininosuccinate lyase (argH). L-arginine can be converted to agmatine by the action of arginine decarboxylase (speA; EC 4.1.1.19), and then to putrescine by the action of agmatine ureohydrolase (speB; EC 3.5.3.11). Also, L-ornithine can be converted to putrescine by the action of ornithine decarboxylase (speC; EC 4.1.1.17). Putrescine can be converted to spermidine by the action of spermidine synthase (speE; EC 2.5.1.16), and then to spermine by the action of spermine synthase (EC 2.5.1.22). Agmatine can also be converted to aminopropylagmatine by the action of agmatine/triamine aminopropyl transferase, and then to spermidine by the action of aminopropylagmatine ureohydrolase. That is, examples of the enzymes that catalyze the conversion of L-arginine or L-ornithine into a polyamine can include these enzymes. Notably, spermidine synthase, spermine synthase, and agmatine/triamine aminopropyl transferase each catalyze the reaction of transferring a propylamine group from decarboxylated S-adenosyl methionine (dcSAM), which can be generated from SAM by decarboxylation, into the corresponding substrate.

Creatine can be produced from L-arginine and glycine. That is, examples of the objective substance biosynthesis enzyme can also include, for example, L-arginine biosynthesis enzymes, glycine biosynthesis enzymes, and enzymes that catalyze the conversion of L-arginine and glycine into creatine. L-arginine and glycine can be combined to generate guanidinoacetate and ornithine by the action of arginine:glycine amidinotransferase (AGAT, EC 2.1.4.1); and guanidinoacetate can be methylated to generate creatine by the action of guanidinoacetate N-methyltransferase (GAMT, EC 2.1.1.2), using SAM as the methyl donor. That is, examples of the enzymes that catalyze the conversion of L-arginine and glycine into creatine can include these enzymes.

Mugineic acid can be produced from SAM. That is, examples of the objective substance biosynthesis enzyme can also include, for example, enzymes that catalyze the conversion of SAM into mugineic acid. One molecule of nicotianamine can be synthesized from three molecules of SAM by the action of nicotianamine synthase (EC 2.5.1.43). Nicotianamine can be converted successively to 3″-deamino-3″-oxonicotianamine, 2′-deoxymugineic-acid, and mugineic-acid by the action of nicotianamine aminotransferase (EC 2.6.1.80), 3″-deamino-3″-oxonicotianamine reductase (EC 1.1.1.285), and 2′-deoxymugineic-acid 2′-dioxygenase (EC 1.14.11.24), respectively. That is, examples of the enzymes that catalyze the conversion of SAM into mugineic acid can include these enzymes.

L-Methionine can be produced from L-cysteine. That is, examples of the objective substance biosynthesis enzyme can also include, for example, L-cysteine biosynthesis enzymes and enzymes that catalyze the conversion of L-cysteine into L-methionine. Examples of the L-cysteine biosynthesis enzymes can include the CysIXHDNYZ proteins, Fpr2 protein, and CysK protein, which are encoded by the cysIXHDNYZ genes, fpr2 gene, and cysK gene, respectively. Examples of the enzymes that catalyze the conversion of L-cysteine into L-methionine can include cystathionine-gamma-synthase and cystathionine-beta-lyase.

Examples of a method for imparting or enhancing an objective substance-producing ability can also include the method of increasing the activity of an uptake system of a substance other than an objective substance, such as a substance generated as an intermediate during production of an objective substance and a substance used as a precursor of an objective substance. That is, the microorganism can be modified so that the activity of such an uptake system is increased. The term “uptake system of a substance” can refer to a protein having a function of incorporating the substance from the outside of a cell into the cell. This activity can also be referred to as an “uptake activity of a substance”. A gene encoding such an uptake system can also be referred to as an “uptake system gene”. Examples of such an uptake system can include a vanillic acid uptake system and a protocatechuic acid uptake system. Examples of the vanillic acid uptake system can include the VanK protein, which is encoded by the vanK gene (M. T. Chaudhry, et al., Microbiology, 2007, 153:857-865). The nucleotide sequence of the vanK gene (NCgl2302) native to the C. glutamicum ATCC 13869 strain is shown as SEQ ID NO: 25, and the amino acid sequence of the VanK protein encoded by this gene is shown as SEQ ID NO: 26. Examples of the protocatechuic acid uptake system gene can include the PcaK protein, which is encoded by the pcaK gene (M. T. Chaudhry, et al., Microbiology, 2007, 153:857-865). The nucleotide sequence of the pcaK gene (NCgl1031) native to the C. glutamicum ATCC 13869 strain is shown as SEQ ID NO: 27, and the amino acid sequence of the PcaK protein encoded by this gene is shown as SEQ ID NO: 28.

The uptake activity of a substance can be measured according to, for example, a known method (M. T. Chaudhry, et al., Microbiology, 2007. 153:857-865).

Examples of the method for imparting or enhancing an objective substance-producing ability further can include a method of reducing the activity of an enzyme that is involved in the by-production of a substance other than an objective substance. Such a substance other than an objective substance can also be referred to as a “byproduct”. Such an enzyme can also be referred to as a “byproduct generation enzyme”. Examples of the byproduct generation enzyme can include, for example, enzymes that are involved in the utilization of an objective substance, and enzymes that catalyze a reaction branching away from the biosynthetic pathway of an objective substance to generate a substance other than the objective substance. The method for reducing the activity of a protein, such as an enzyme etc., will be described herein. The activity of a protein, such as an enzyme etc., can be reduced by, for example, disrupting a gene that encodes the protein. For example, it has been reported that, in coryneform bacteria, vanillin is metabolized in the order of vanillin−>vanillic acid−> protocatechuic acid, and utilized (Current Microbiology, 2005, Vol. 51, pp. 59-65). That is, specific examples of the byproduct generation enzyme can include an enzyme that catalyzes the conversion of vanillin into protocatechuic acid and enzymes that catalyze further metabolization of protocatechuic acid. Examples of such enzymes can include vanillate demethylase, protocatechuate 3,4-dioxygenase, and various enzymes that further decompose the reaction product of protocatechuate 3,4-dioxygenase to succinyl-CoA and acetyl-CoA (Appl. Microbiol. Biotechnol., 2012, Vol. 95, p77-89). In addition, vanillin can be converted into vanillyl alcohol by the action of alcohol dehydrogenase (Kunjapur A M. et al., J. Am. Chem. Soc., 2014, Vol. 136, p11644-11654.; Hansen E H. et al., App. Env. Microbiol., 2009, Vol. 75, p2765-2774.). That is, specific examples of the byproduct generation enzyme can also include alcohol dehydrogenase (ADH). In addition, 3-dehydroshikimic acid, which is an intermediate of the biosynthetic pathway of vanillic acid and vanillin, can also be converted into shikimic acid by the action of shikimate dehydrogenase. That is, specific examples of the byproduct generation enzyme can also include shikimate dehydrogenase.

The term “vanillate demethylase” can refer to a protein having an activity for catalyzing the reaction of demethylating vanillic acid to generate protocatechuic acid. This activity can also be referred to as “vanillate demethylase activity”. A gene encoding vanillate demethylase can also be referred to as a “vanillate demethylase gene”. Examples of vanillate demethylase can include the VanAB proteins, which are encoded by the vanAB genes (Current Microbiology, 2005, Vol. 51, pp. 59-65). The vanA gene and vanB gene encode the subunit A and subunit B of vanillate demethylase, respectively. To reduce the vanillate demethylase activity, both the vanAB genes may be disrupted or the like, or only one of the two may be disrupted or the like. The nucleotide sequences of the vanAB genes native to the C. glutamicum ATCC 13869 strain are shown as SEQ ID NOS: 29 and 31, and the amino acid sequences of the VanAB proteins encoded by these genes are shown as SEQ ID NOS: 30 and 32, respectively. The vanAB genes usually constitute the vanABK operon together with the vanK gene. Therefore, in order to reduce the vanillate demethylase activity, the vanABK operon may be totally disrupted or the like, for example, deleted. In such a case, the vanK gene may be introduced to a host again. For example, when vanillic acid present outside cells is used, and the vanABK operon is totally disrupted or the like, for example, deleted, it is preferable to introduce the vanK gene anew.

The vanillate demethylase activity can be measured by, for example, incubating the enzyme with a substrate, such as vanillic acid, and measuring the enzyme- and substrate-dependent generation of protocatechuic acid (J Bacteriol, 2001, Vol. 183, p 3276-3281).

The term “protocatechuate 3,4-dioxygenase” can refer to a protein having an activity for catalyzing the reaction of oxidizing protocatechuic acid to generate beta-Carboxy-cis,cis-muconic acid. This activity can also be referred to as “protocatechuate 3,4-dioxygenase activity”. A gene encoding protocatechuate 3,4-dioxygenase can also be referred to as a “protocatechuate 3,4-dioxygenase gene”. Examples of protocatechuate 3,4-dioxygenase can include the PcaGH proteins, which are encoded by the pcaGH genes (Appl. Microbiol. Biotechnol., 2012, Vol. 95, p 77-89). The pcaG gene and pcaH gene encode the alpha subunit and beta subunit of protocatechuate 3,4-dioxygenase, respectively. To reduce the protocatechuate 3,4-dioxygenase activity, both the pcaGH genes may be disrupted or the like, or only one of the two may be disrupted or the like. The nucleotide sequences of the pcaGH genes native to the C. glutamicum ATCC 13032 strain are shown as SEQ ID NOS: 33 and 35, and the amino acid sequences of the PcaGH proteins encoded by these genes are shown as SEQ ID NOS: 34 and 36, respectively.

The protocatechuate 3,4-dioxygenase activity can be measured by, for example, incubating the enzyme with a substrate, such as protocatechuic acid, and measuring the enzyme- and substrate-dependent oxygen consumption (Meth. Enz., 1970, Vol. 17A, p 526-529).

The term “alcohol dehydrogenase (ADH)” can refer to a protein that has an activity for catalyzing the reaction of reducing an aldehyde in the presence of an electron donor to generate an alcohol (EC 1.1.1.1, EC 1.1.1.2, EC 1.1.1.71, etc.). This activity can also be referred to as “ADH activity”. A gene encoding ADH can also be referred to as an “ADH gene”. Examples of the electron donor can include NADH and NADPH.

As ADH, one having an activity for catalyzing the reaction of reducing vanillin in the presence of an electron donor to generate vanillyl alcohol is a particular example. This activity can also be especially referred to as “vanillyl alcohol dehydrogenase activity”. Furthermore, ADH having the vanillyl alcohol dehydrogenase activity can also be especially referred to as “vanillyl alcohol dehydrogenase”.

Examples of ADH can include the YqhD protein, NCgl0324 protein, NCgl0313 protein, NCgl2709 protein, NCgl0219 protein, and NCgl2382 protein, which are encoded by the yqhD gene, NCgl0324 gene, NCgl0313 gene, NCgl2709 gene, NCgl0219 gene, and NCgl2382 gene, respectively. The yqhD gene and the NCgl0324 gene encode vanillyl alcohol dehydrogenase. The yqhD gene can be found in, for example, bacteria belonging to the family Enterobacteriaceae such as E. coli. The NCgl0324 gene, NCgl0313 gene, NCgl2709 gene, NCgl0219 gene, and NCgl2382 gene can be found in, for example, coryneform bacteria such as C. glutamicum. The nucleotide sequence of the yqhD gene native to E. coli K-12 MG1655 strain is shown as SEQ ID NO: 37, and the amino acid sequence of the YqhD protein encoded by this gene is shown as SEQ ID NO: 38. The nucleotide sequences of the NCgl0324 gene, NCgl0313 gene, and NCgl2709 gene native to the C. glutamicum ATCC 13869 strain are shown as SEQ ID NOS: 39, 41, and 43, respectively, and the amino acid sequences of the proteins encoded by these genes are shown as SEQ ID NOS: 40, 42, and 44, respectively. The nucleotide sequences of the NCgl0219 gene and NCgl2382 gene native to the C. glutamicum ATCC 13032 strain are shown as SEQ ID NOS: 45 and 47, respectively, and the amino acid sequences of the proteins encoded by these genes are shown as SEQ ID NOS: 46 and 48, respectively. The activity of one kind of ADH may be reduced, or the activities of two or more kinds of ADHs may be reduced. For example, the activity or activities of one or more of the NCgl0324 protein, NCgl2709 protein, and NCgl0313 protein may be reduced. Particularly, at least the activity of NCgl0324 protein may be reduced.

The ADH activity can be measured by, for example, incubating the enzyme with a substrate, such as an aldehyde such as vanillin, in the presence of NADPH or NADH, and measuring the enzyme- and substrate-dependent oxidation of NADPH or NADH.

The term “shikimate dehydrogenase” can refer to a protein that has the activity of catalyzing the reaction of reducing 3-dehydroshikimic acid in the presence of an electron donor to generate shikimic acid (EC 1.1.1.25). This activity can also be referred to as “shikimate dehydrogenase activity”. A gene encoding shikimate dehydrogenase can also be referred to as a “shikimate dehydrogenase gene”. Examples of the electron donor can include NADH and NADPH. Examples of a shikimate dehydrogenase can include the AroE protein, which is encoded by the aroE gene. The nucleotide sequence of the aroE gene native to the E. coli K-12 MG1655 strain is shown as SEQ ID NO: 49, and the amino acid sequence of the AroE protein encoded by this gene is shown as SEQ ID NO: 50.

The shikimate dehydrogenase activity can be measured by, for example, incubating the enzyme with a substrate, such as 3-dehydroshikimic acid in the presence of NADPH or NADH, and measuring the enzyme- and substrate-dependent oxidation of NADPH or NADH.

The protein of which the activity is to be modified can be appropriately chosen depending on the type of biosynthesis pathway via which an objective substance is produced and on the types and activities of the proteins inherently present in the chosen microorganism. For example, when vanillin is produced by bioconversion of protocatechuic acid, it may be preferable to enhance the activity or activities of one or more of OMT, ACAR, PPT, and the protocatechuic acid uptake system. Also, when vanillin is produced by bioconversion of protocatechualdehyde, it may be preferable to enhance the activity of OMT.

The genes and proteins used for breeding a microorganism having an objective substance-producing ability may have, for example, the above-exemplified or other known nucleotide sequences and amino acid sequences, respectively. Also, the genes and proteins used for breeding a microorganism having an objective substance-producing ability may be conservative variants of the genes and proteins exemplified above, such as genes and proteins having the above-exemplified or other known nucleotide sequences and amino acid sequences, respectively. Specifically, for example, the genes used for breeding a microorganism having an objective substance-producing ability may each be a gene encoding a protein having the amino acid sequence exemplified above or the amino acid sequence of a known protein, but which can include substitution, deletion, insertion, and/or addition of one or several some amino acid residues at one or several positions, so long as the original function of the protein, such as its enzymatic activity, transporter activity, etc., is maintained. As for conservative variants of genes and proteins, the descriptions concerning conservative variants of NCgl2048 gene and NCgl2048 protein described later can be applied mutatis mutandis.

### <1-2> Reduction in Activity of NCgl2048 Protein

The microorganism can be modified so that the activity of the NCgl2048 protein is reduced. Specifically, the microorganism can be modified so that the activity of the NCgl2048 protein is reduced as compared with a non-modified strain. By modifying a microorganism so that the activity of NCgl2048 protein is reduced, an objective substance-producing ability of the microorganism can be improved, and that is, the production of an objective substance by using the microorganism can be increased. Also, by modifying a microorganism so that the activity of NCgl2048 protein is reduced, an ability of the microorganism for generating or regenerating SAM may possibly be improved. That is, specifically, an increase in an objective substance-producing ability of a microorganism may be due to an increase in an ability of the microorganism for generating or regenerating SAM.

The microorganism can be obtained by modifying a microorganism having an objective substance-producing ability so that the activity of NCgl2048 protein is reduced. The microorganism can also be obtained by modifying a microorganism so that the activity of NCgl2048 protein is reduced, and then imparting an objective substance-producing ability to the microorganism or enhancing an objective substance-producing ability of the microorganism. In addition, the microorganism may have acquired an objective substance-producing ability as a result of a modification that reduces the activity of NCgl2048 protein, or as a result of a combination of a modification that reduces the activity of NCgl2048 protein and other modification(s) for imparting or enhancing an objective substance-producing ability. The modifications for constructing the microorganism can be performed in an arbitrary order.

The term “NCgl2048 protein” can refer to a protein encoded by the NCgl2048 gene. Examples of the NCgl2048 protein can include those native to various organisms such as Enterobacteriaceae bacteria and coryneform bacteria. Specific examples of the NCgl2048 protein can include the NCgl2048 protein native to C. glutamicum. The nucleotide sequence of the NCgl2048 gene native to the C. glutamicum ATCC 13869 strain is shown as SEQ ID NO: 92, and the amino acid sequence of the protein encoded by this gene is shown as SEQ ID NO: 93.

That is, the NCgl2048 gene may be, for example, a gene having the nucleotide sequence shown as SEQ ID NO: 92. Also, NCgl2048 protein may be, for example, a protein having the amino acid sequence shown as SEQ ID NO: 93. The expression “a gene or protein has a nucleotide or amino acid sequence” encompasses when a gene or protein includes the nucleotide or amino acid sequence, and when a gene or protein includes only the nucleotide or amino acid sequence.

The NCgl2048 gene may be a variant of any of the NCgl2048 genes exemplified above, that is, a gene having the nucleotide sequence shown as SEQ ID NO: 92, so long as the original function thereof is maintained. Similarly, the NCgl2048 protein may be a variant of any of the NCgl2048 proteins exemplified above, that is, a protein having the amino acid sequence shown as SEQ ID NO: 93, so long as the original function thereof is maintained. A variant that maintains the original function thereof can also be referred to as a “conservative variant”. The term “NCgl2048 gene” can include not only the NCgl2048 gene exemplified above, such as the NCgl2048 gene having the nucleotide sequence shown as SEQ ID NO: 92, but can also include conservative variants thereof. Similarly, the term “NCgl2048 protein” can include not only the NCgl2048 protein exemplified above, such as the NCgl2048 protein having the amino acid sequence shown as SEQ ID NO: 93, but can also include conservative variants thereof. Examples of the conservative variants can include, for example, homologues and artificially modified versions of the genes and proteins exemplified above.

The expression “the original function is maintained” means that a variant of a gene or protein has a function, such as activity or property, corresponding to the function, such as activity or property, of the original gene or protein. The expression “the original function is maintained” when referring to a gene means that a variant of the gene encodes a protein that maintains the original function. That is, the expression “the original function is maintained” when referring to the NCgl2048 gene means that the variant of the gene encodes a protein having the function of NCgl2048 protein, such as the function of the protein consisting of the amino acid sequence shown as SEQ ID NO: 93. The expression “the original function is maintained” when referring to the NCgl2048 gene may also mean that the variant of the gene has a property that a reduction in the expression of the gene in a microorganism provides an increased production of an objective substance. The expression “the original function is maintained” when referring to the NCgl2048 protein means that the variant of the protein has the function of NCgl2048 protein, such as the function of the protein having the amino acid sequence shown as SEQ ID NO: 93. The expression “the original function is maintained” when referring to the NCgl2048 protein may also mean that the variant of the protein has a property that a reduction in the activity of the protein in a microorganism provides an increased production of an objective substance.

Hereafter, examples of the conservative variants will be explained.

Homologues of the NCgl2048 gene or NCgl2048 protein can be easily obtained from public databases by, for example, BLAST search or FASTA search using any of the nucleotide sequences of the NCgl2048 genes exemplified above or any of the amino acid sequences of NCgl2048 proteins exemplified above as a query sequence. Furthermore, homologues of the NCgl2048 gene can be obtained by, for example, PCR using a chromosome of an organism such as coryneform bacteria as the template, and oligonucleotides prepared on the basis of any of the nucleotide sequences of the NCgl2048 genes exemplified above as primers.

The NCgl2048 gene may encode a protein having any of the aforementioned amino acid sequences, such as the amino acid sequence shown as SEQ ID NO: 93, but that includes substitution, deletion, insertion, and/or addition of one or several amino acid residues at one or several positions, so long as the original function is maintained. For example, the encoded protein may have an extended or deleted N-terminus and/or C-terminus. Although the number meant by the term “one or several” used above may differ depending on the positions of amino acid residues in the three-dimensional structure of the protein or the types of amino acid residues, specifically, it is, for example, 1 to 50, 1 to 40, 1 to 30, 1 to 20, 1 to 10, 1 to 5, or 1 to 3.

The aforementioned substitution, deletion, insertion, and/or addition of one or several amino acid residues can each be a conservative mutation that maintains the original function of the protein. Typical examples of the conservative mutation are conservative substitutions. The conservative substitution is a mutation wherein substitution takes place mutually among Phe, Trp, and Tyr, if the substitution site is an aromatic amino acid; among Leu, Ile, and Val, if it is a hydrophobic amino acid; between Gln and Asn, if it is a polar amino acid; among Lys, Arg, and His, if it is a basic amino acid; between Asp and Glu, if it is an acidic amino acid; and between Ser and Thr, if it is an amino acid having a hydroxyl group. Examples of substitutions considered as conservative substitutions can include, specifically, substitution of Ser or Thr for Ala, substitution of Gln, His, or Lys for Arg, substitution of Glu, Gln, Lys, His, or Asp for Asn, substitution of Asn, Glu, or Gln for Asp, substitution of Ser or Ala for Cys, substitution of Asn, Glu, Lys, His, Asp, or Arg for Gln, substitution of Gly, Asn, Gln, Lys, or Asp for Glu, substitution of Pro for Gly, substitution of Asn, Lys, Gln, Arg, or Tyr for His, substitution of Leu, Met, Val, or Phe for Ile, substitution of Ile, Met, Val, or Phe for Leu, substitution of Asn, Glu, Gln, His, or Arg for Lys, substitution of Ile, Leu, Val, or Phe for Met, substitution of Trp, Tyr, Met, Ile, or Leu for Phe, substitution of Thr or Ala for Ser, substitution of Ser or Ala for Thr, substitution of Phe or Tyr for Trp, substitution of His, Phe, or Trp for Tyr, and substitution of Met, Ile, or Leu for Val. Furthermore, such substitution, deletion, insertion, addition, or the like of amino acid residues as mentioned above can include a naturally occurring mutation due to an individual difference, or a difference of species of the organism from which the gene is derived (mutant or variant).

Furthermore, the NCgl2048 gene may be a gene encoding a protein having an amino acid sequence having a homology of, for example, 50% or more, 65% or more, 80% or more, 90% or more, 95% or more, 97% or more, or 99% or more, to the total amino acid sequence of any of the aforementioned amino acid sequences, so long as the original function is maintained. In addition, in this specification, “homology” is equivalent to “identity”.

Furthermore, the NCgl2048 gene may be a gene, such as a DNA, that is able to hybridize under stringent conditions with a probe that can be prepared from any of the aforementioned nucleotide sequences, such as the nucleotide sequence shown as SEQ ID NO: 92, for example, with a sequence complementary to the whole sequence or a partial sequence of any of the aforementioned nucleotide sequences, so long as the original function is maintained. The “stringent conditions” can refer to conditions under which a so-called specific hybrid is formed, and a non-specific hybrid is not formed. Examples of the stringent conditions can include those under which highly homologous DNAs hybridize to each other, for example, DNAs not less than 50%, 65%, or 80% homologous, not less than 90% homologous, not less than 95% homologous, not less than 97% homologous, or not less than 99% homologous, hybridize to each other, and DNAs less homologous than the above do not hybridize to each other, or conditions of washing of typical Southern hybridization, that is, conditions of washing once, or 2 or 3 times, at a salt concentration and temperature corresponding to 1×SSC, 0.1% SDS at 60° C.; 0.1×SSC, 0.1% SDS at 60° C.; or 0.1×SSC, 0.1% SDS at 68° C.

The probe used for the aforementioned hybridization may be a part of a sequence that is complementary to the gene as described above. Such a probe can be prepared by PCR using oligonucleotides prepared on the basis of a known gene sequence as primers and a DNA fragment containing any of the aforementioned genes as a template. As the probe, for example, a DNA fragment having a length of about 300 bp can be used. When a DNA fragment having a length of about 300 bp is used as the probe, in particular, the washing conditions of the hybridization may be, for example, 50° C., 2×SSC and 0.1% SDS.

Furthermore, since properties concerning the degeneracy of codons changes depending on the host, the NCgl2048 gene can include substitution of respective equivalent codons for arbitrary codons. That is, NCgl2048 gene may be a variant of any of the NCgl2048 genes exemplified above due to the degeneracy of the genetic code. For example, NCgl2048 gene may be a gene modified so that it has optimal codons according to codon frequencies in the chosen host.

The percentage of the sequence identity between two sequences can be determined by, for example, a mathematical algorithm. Non-limiting examples of such a mathematical algorithm can include the algorithm of Myers and Miller (1988) CABIOS 4:11-17, the local homology algorithm of Smith et al (1981) Adv. Appl. Math. 2:482, the homology alignment algorithm of Needleman and Wunsch (1970) J. Mol. Biol. 48:443-453, the method for searching homology of Pearson and Lipman (1988) Proc. Natl. Acad. Sci. 85:2444-2448, and a modified version of the algorithm of Karlin and Altschul (1990) Proc. Natl. Acad. Sci. USA 87:2264, such as that described in Karlin and Altschul (1993) Proc. Natl. Acad. Sci. USA 90:5873-5877.

By using a program based on such a mathematical algorithm, sequence comparison, and an alignment for determining the sequence identity can be performed. The program can be appropriately executed by a computer. Examples of such a program can include, but are not limited to, CLUSTAL of PC/Gene program (available from Intelligenetics, Mountain View, Calif.), ALIGN program (Version 2.0), and GAP, BESTFIT, BLAST, FASTA, and TFASTA of Wisconsin Genetics Software Package, Version 8 (available from Genetics Computer Group (GCG), 575 Science Drive, Madison, Wis., USA). Alignment using these programs can be performed by using, for example, initial parameters. The CLUSTAL program is well described in Higgins et al. (1988) Gene 73:237-244 (1988), Higgins et al. (1989) CABIOS 5:151-153, Corpet et al. (1988) Nucleic Acids Res. 16:10881-90, Huang et al. (1992) CABIOS 8:155-65, and Pearson et al. (1994) Meth. Mol. Biol. 24:307-331.

In order to obtain a nucleotide sequence homologous to a target nucleotide sequence, in particular, for example, BLAST nucleotide search can be performed by using BLASTN program with score of 100 and word length of 12. In order to obtain an amino acid sequence homologous to a target protein, in particular, for example, BLAST protein search can be performed by using BLASTX program with score of 50 and word length of 3. See ncbi.nlm.nih.gov for BLAST nucleotide search and BLAST protein search. In addition, Gapped BLAST (BLAST 2.0) can be used in order to obtain an alignment including gap(s) for the purpose of comparison. In addition, PSI-BLAST can be used in order to perform repetitive search for detecting distant relationships between sequences. See Altschul et al. (1997) Nucleic Acids Res. 25:3389 for Gapped BLAST and PSI-BLAST. When using BLAST, Gapped BLAST, or PSI-BLAST, initial parameters of each program (e.g. BLASTN for nucleotide sequences, and BLASTX for amino acid sequences) can be used. Alignment can also be manually performed.

The sequence identity between two sequences is calculated as the ratio of residues matching in the two sequences when aligning the two sequences so as to fit maximally with each other. The term “identity” between amino acid sequences may specifically mean an identity calculated by blastp with default scoring parameters (i.e. Matrix, BLOSUM62; Gap Costs, Existence=11, Extension=1; Compositional Adjustments, Conditional compositional score matrix adjustment), unless otherwise stated. The term “identity” between nucleotide sequences may specifically mean an identity calculated by blastn with default scoring parameters (i.e. Match/Mismatch Scores=1, -2; Gap Costs=Linear), unless otherwise stated.

The aforementioned descriptions concerning conservative variants of the genes and proteins can be applied mutatis mutandis to variants of arbitrary proteins such as objective substance biosynthesis enzymes and genes encoding them.

### <1-3> Methods for Increasing Activity of Protein

Hereafter, the methods for increasing the activity of a protein will be explained.

The expression “the activity of a protein is increased” means that the activity of the protein is increased as compared with a non-modified strain. Specifically, the expression “the activity of a protein is increased” can mean that the activity of the protein per cell is increased as compared with that of a non-modified strain. The term “non-modified strain” or “strain of a non-modified microorganism” can refer to a control strain that has not been modified so that the activity of an objective protein is increased. Examples of the non-modified strain can include a wild-type strain and parent strain. Specific examples of the non-modified strain can include the respective type strains of the species of microorganisms. Specific examples of the non-modified strain can also include strains exemplified above in relation to the description of microorganisms. That is, in an embodiment, the activity of a protein may be increased as compared with a type strain, i.e. the type strain of the species to which a microorganism belongs. In another embodiment, the activity of a protein may also be increased as compared with the C. glutamicum ATCC 13869 strain. In another embodiment, the activity of a protein may also be increased as compared with the C. glutamicum ATCC 13032 strain. In another embodiment, the activity of a protein may also be increased as compared with the E. coli K-12 MG1655 strain.

The phrase “the activity of a protein is increased” may also be expressed as “the activity of a protein is enhanced”. More specifically, the expression “the activity of a protein is increased” may mean that the number of molecules of the protein per cell is increased, and/or the function of each molecule of the protein is increased as compared with those of a non-modified strain. That is, the term “activity” in the expression “the activity of a protein is increased” is not limited to the catalytic activity of the protein, but may also mean the transcription amount of a gene, that is, the amount of mRNA, encoding the protein, or the translation amount of the protein, that is, the amount of the protein. Furthermore, the phrase “the activity of a protein is increased” can include not only when the activity of an objective protein is increased in a strain inherently having the activity of the objective protein, but also when the activity of an objective protein is imparted to a strain not inherently having the activity of the objective protein. Furthermore, so long as the activity of the protein is eventually increased, the activity of an objective protein inherently present in a host may be attenuated and/or eliminated, and then an appropriate type of the objective protein may be imparted to the host.

The degree of the increase in the activity of a protein is not particularly limited, so long as the activity of the protein is increased as compared with a non-modified strain. The activity of the protein may be increased to, for example, 1.2 times or more, 1.5 times or more, 2 times or more, or 3 times or more of that of a non-modified strain. Furthermore, when the non-modified strain does not have the activity of the objective protein, it is sufficient that the protein is produced as a result of introduction of the gene encoding the protein, and for example, the protein may be produced to such an extent that the activity thereof can be measured.

The modification for increasing the activity of a protein can be attained by, for example, increasing the expression of a gene encoding the protein. The phrase “the expression of a gene is increased” means that the expression of the gene is increased as compared with a non-modified strain, such as a wild-type strain and parent strain. Specifically, the phrase “the expression of a gene is increased” may mean that the expression amount of the gene per cell is increased as compared with that of a non-modified strain. More specifically, the phrase “the expression of a gene is increased” may mean that the transcription amount of the gene, that is, the amount of mRNA, is increased, and/or the translation amount of the gene, that is, the amount of the protein expressed from the gene, is increased. The phrase “the expression of a gene is increased” can also be referred to as “the expression of a gene is enhanced”. The expression of a gene may be increased to, for example, 1.2 times or more, 1.5 times or more, 2 times or more, or 3 times or more of that of a non-modified strain. Furthermore, the phrase “the expression of a gene is increased” can include not only when the expression amount of an objective gene is increased in a strain that inherently expresses the objective gene, but also when the gene is introduced into a strain that does not inherently express the objective gene, and is expressed therein. That is, the phrase “the expression of a gene is increased” may also mean, for example, that an objective gene is introduced into a strain that does not possess the gene, and is expressed therein.

The expression of a gene can be increased by, for example, increasing the copy number of the gene.

The copy number of a gene can be increased by introducing the gene into the chromosome of a host. A gene can be introduced into a chromosome by, for example, using homologous recombination (Miller, J. H., Experiments in Molecular Genetics, 1972, Cold Spring Harbor Laboratory). Examples of the gene transfer method utilizing homologous recombination can include, for example, a method of using a linear DNA such as Red-driven integration (Datsenko, K. A., and Wanner, B. L., Proc. Natl. Acad. Sci. USA, 97:6640-6645 (2000)), a method of using a plasmid containing a temperature sensitive replication origin, a method of using a plasmid capable of conjugative transfer, a method of using a suicide vector not having a replication origin that functions in a host, and a transduction method using a phage. Only one copy of a gene may be introduced, or two or more copies of a gene may be introduced. For example, by performing homologous recombination using a sequence which is present in multiple copies on a chromosome as a target, multiple copies of a gene can be introduced into the chromosome. Examples of such a sequence which is present in multiple copies on a chromosome can include repetitive DNAs, and inverted repeats located at the both ends of a transposon. Alternatively, homologous recombination may be performed by using an appropriate sequence on a chromosome, such as a gene, unnecessary for the production of an objective substance as a target. Furthermore, a gene can also be randomly introduced into a chromosome by using a transposon or Mini-Mu (Japanese Patent Laid-open (Kokai) No. 2-109985, U.S. Pat. No. 5,882,888, EP 805867 B1).

Introduction of a target gene into a chromosome can be confirmed by Southern hybridization using a probe having a sequence complementary to the whole gene or a part thereof, PCR using primers prepared on the basis of the sequence of the gene, or the like.

Furthermore, the copy number of a gene can also be increased by introducing a vector containing the gene into a host. For example, the copy number of a target gene can be increased by ligating a DNA fragment containing the target gene with a vector that functions in a host to construct an expression vector of the gene, and transforming the host with the expression vector. The DNA fragment containing the target gene can be obtained by, for example, PCR using the genomic DNA of a microorganism having the target gene as the template. As the vector, a vector autonomously replicable in the cell of the host can be used. The vector can be a multi-copy vector. Furthermore, the vector can have a marker such as an antibiotic resistance gene for selection of transformant. Furthermore, the vector can have a promoter and/or terminator for expressing the introduced gene. The vector may be, for example, a vector derived from a bacterial plasmid, a vector derived from a yeast plasmid, a vector derived from a bacteriophage, cosmid, phagemid, or the like. Specific examples of a vector autonomously replicable in Enterobacteriaceae bacteria such as Escherichia coli can include, for example, pUC19, pUC18, pHSG299, pHSG399, pHSG398, pBR322, pSTV29 (all of these are available from Takara Bio), pACYC184, pMW219 (NIPPON GENE), pTrc99A (Pharmacia), pPROK series vectors (Clontech), pKK233-2 (Clontech), pET series vectors (Novagen), pQE series vectors (QIAGEN), pCold TF DNA (TaKaRa), pACYC series vectors, and the broad host spectrum vector RSF1010. Specific examples of a vector autonomously replicable in coryneform bacteria can include, for example, pHM1519 (Agric. Biol. Chem., 48, 2901-2903 (1984)); pAM330 (Agric. Biol. Chem., 48, 2901-2903 (1984)); plasmids obtained by improving these and having a drug resistance gene; plasmid pCRY30 described in Japanese Patent Laid-open (Kokai) No. 3-210184; plasmids pCRY21, pCRY2KE, pCRY2KX, pCRY31, pCRY3KE, and pCRY3KX described in Japanese Patent Laid-open (Kokai) No. 2-72876 and U.S. Pat. No. 5,185,262; plasmids pCRY2 and pCRY3 described in Japanese Patent Laid-open (Kokai) No. 1-191686; pAJ655, pAJ611, and pAJ1844 described in Japanese Patent Laid-open (Kokai) No. 58-192900; pCG1 described in Japanese Patent Laid-open (Kokai) No. 57-134500; pCG2 described in Japanese Patent Laid-open (Kokai) No. 58-35197; pCG4 and pCG11 described in Japanese Patent Laid-open (Kokai) No. 57-183799; pVK7 described in Japanese Patent Laid-open (Kokai) No. 10-215883; pVK9 described in WO2007/046389; pVS7 described in WO2013/069634; and pVC7 described in Japanese Patent Laid-open (Kokai) No. 9-070291.

When a gene is introduced, it is sufficient that the gene can be expressed by a host. Specifically, it is sufficient that the gene is present in a host so that it is expressed under control of a promoter that functions in the host. The term “a promoter that functions in a host” can refer to a promoter that shows a promoter activity in the host. The promoter may be a promoter derived from the host, or a heterogenous promoter. The promoter may be the native promoter of the gene to be introduced, or a promoter of another gene. As the promoter, for example, such a stronger promoter as described herein may also be used.

A terminator for termination of gene transcription may be located downstream of the gene. The terminator is not particularly limited so long as it functions in the chosen host. The terminator may be a terminator derived from the host, or a heterogenous terminator. The terminator may be the native terminator of the gene to be introduced, or a terminator of another gene. Specific examples of the terminator can include, for example, T7 terminator, T4 terminator, fd phage terminator, tet terminator, and trpA terminator.

Vectors, promoters, and terminators available in various microorganisms are disclosed in detail in “Fundamental Microbiology Vol. 8, Genetic Engineering, KYORITSU SHUPPAN CO., LTD, 1987”, and those can be used.

Furthermore, when two or more of genes are introduced, it is sufficient that the genes each can be expressed by a host. For example, all the genes may be carried by a single expression vector or a chromosome. Furthermore, the genes may be separately carried by two or more expression vectors, or separately carried by a single or two or more expression vectors and a chromosome. An operon constituted by two or more genes may also be introduced. The phrase “introducing two or more genes” can mean, for example, introducing respective genes encoding two or more kinds of proteins, such as enzymes, introducing respective genes encoding two or more subunits constituting a single protein complex, such as an enzyme complex, and a combination thereof.

The gene to be introduced is not particularly limited so long as it encodes a protein that functions in the host. The gene to be introduced may be a gene derived from the host, or may be a heterogenous gene. The gene to be introduced can be obtained by, for example, PCR using primers designed on the basis of the nucleotide sequence of the gene, and using the genomic DNA of an organism having the gene, a plasmid carrying the gene, or the like as a template. The gene to be introduced may also be totally synthesized, for example, on the basis of the nucleotide sequence of the gene (Gene, 60(1), 115-127 (1987)). The obtained gene can be used as it is, or after being modified as required. That is, a variant of a gene may be obtained by modifying the gene. A gene can be modified by a known technique. For example, an objective mutation can be introduced into an objective site of DNA by the site-specific mutation method. That is, the coding region of a gene can be modified by the site-specific mutation method so that a specific site of the encoded protein includes substitution, deletion, insertion, and/or addition of amino acid residues. Examples of the site-specific mutation method can include the method utilizing PCR (Higuchi, R., 61, in PCR Technology, Erlich, H. A. Eds., Stockton Press (1989); Carter, P., Meth. in Enzymol., 154, 382 (1987)), and the method utilizing phage (Kramer, W. and Frits, H. J., Meth. in Enzymol., 154, 350 (1987); Kunkel, T. A. et al., Meth. in Enzymol., 154, 367 (1987)). Alternatively, a variant of a gene may be totally synthesized.

In addition, when a protein functions as a complex made up of a plurality of subunits, a part or all of the plurality of subunits may be modified, so long as the activity of the protein is eventually increased. That is, for example, when the activity of a protein is increased by increasing the expression of a gene, the expression of a part or all of the plurality of genes that encode the subunits may be enhanced. It is usually preferable to enhance the expression of all of the plurality of genes encoding the subunits. Furthermore, the subunits constituting the complex may be derived from a single kind of organism or two or more kinds of organisms, so long as the complex has a function of the objective protein. That is, for example, genes of the same organism encoding a plurality of subunits may be introduced into a host, or genes of different organisms encoding a plurality of subunits may be introduced into a host.

Furthermore, the expression of a gene can be increased by improving the transcription efficiency of the gene. In addition, the expression of a gene can also be increased by improving the translation efficiency of the gene. The transcription efficiency of the gene and the translation efficiency of the gene can be improved by, for example, modifying an expression control sequence of the gene. The term “expression control sequence” collectively can refer to sites that affect the expression of a gene. Examples of the expression control sequence can include, for example, a promoter, a Shine-Dalgarno (SD) sequence, which can also be referred to as ribosome binding site (RBS), and a spacer region between RBS and the start codon. Expression control sequences can be identified by using a promoter search vector or gene analysis software such as GENETYX. These expression control sequences can be modified by, for example, a method of using a temperature sensitive vector, or the Red driven integration method (WO2005/010175).

The transcription efficiency of a gene can be improved by, for example, replacing the promoter of the gene on a chromosome with a stronger promoter. The term “stronger promoter” can refer to a promoter providing an improved transcription of a gene compared with the inherent wild-type promoter of the gene. Examples of stronger promoters can include, for example, the known high expression promoters such as T7 promoter, trp promoter, lac promoter, thr promoter, tac promoter, trc promoter, tet promoter, araBAD promoter, rpoH promoter, msrA promoter, Pm1 promoter (derived from the genus Bifidobacterium), PR promoter, and PL promoter. Examples of stronger promoters usable in coryneform bacteria can include, for example, the artificially modified P54-6 promoter (Appl. Microbiol. Biotechnol., 53, 674-679 (2000)), pta, aceA, aceB, adh, and amyE promoters inducible in coryneform bacteria with acetic acid, ethanol, pyruvic acid, or the like, cspB, SOD, and tuf (EF-Tu) promoters, which are potent promoters capable of providing a large expression amount in coryneform bacteria (Journal of Biotechnology, 104 (2003) 311-323; Appl. Environ. Microbiol., 2005 December; 71 (12):8587-96), P2 promoter (position 942-1034 of SEQ ID NO: 81), and P3 promoter (SEQ ID NO: 84), as well as lac promoter, tac promoter, and trc promoter. Furthermore, as the stronger promoter, a highly-active type of an existing promoter may also be obtained by using various reporter genes. For example, by making the -35 and -10 regions in a promoter region closer to the consensus sequence, the activity of the promoter can be enhanced (WO00/18935). Examples of a highly active-type promoter can include various tac-like promoters (Katashkina J I et al., Russian Federation Patent Application No. 2006134574). Methods for evaluating the strength of promoters and examples of strong promoters are described in the paper of Goldstein et al. (Prokaryotic Promoters in Biotechnology, Biotechnol. Annu. Rev., 1, 105-128 (1995)), and so forth.

The translation efficiency of a gene can be improved by, for example, replacing the Shine-Dalgarno (SD) sequence, which can also be referred to as ribosome binding site (RBS), for the gene on a chromosome with a stronger SD sequence. The term “stronger SD sequence” can refer to a SD sequence that provides an improved translation of mRNA compared with the inherent wild-type SD sequence of the gene. Examples of stronger SD sequences can include, for example, RBS of the gene 10 derived from phage T7 (Olins P. O. et al, Gene, 1988, 73, 227-235). Furthermore, it is known that substitution, insertion, or deletion of several nucleotides in a spacer region between RBS and the start codon, especially in a sequence immediately upstream of the start codon (5′-UTR), significantly affects the stability and translation efficiency of mRNA, and hence, the translation efficiency of a gene can also be improved by modification.

The translation efficiency of a gene can also be improved by, for example, modifying codons. For example, the translation efficiency of the gene can be improved by replacing a rare codon present in the gene with a more frequently used synonymous codon. That is, the gene to be introduced may be modified, for example, so as to contain optimal codons according to the frequencies of codons observed in the chosen host. Codons can be replaced by, for example, the site-specific mutation method for introducing an objective mutation into an objective site of DNA. Alternatively, a gene fragment in which objective codons are replaced may be entirely synthesized. Frequencies of codons in various organisms are disclosed in the “Codon Usage Database” (kazusa.or.jp/codon; Nakamura, Y. et al, Nucl. Acids Res., 28, 292 (2000)).

Furthermore, the expression of a gene can also be increased by amplifying a regulator that increases the expression of the gene, or deleting or attenuating a regulator that reduces the expression of the gene.

Such methods for increasing the gene expression as described above may be used independently or in an arbitrary combination.

Furthermore, the modification that increases the activity of a protein can also be attained by, for example, enhancing the specific activity of the enzyme. Enhancement of the specific activity can also include desensitization to feedback inhibition. That is, when a protein is subject to feedback inhibition by a metabolite, the activity of the protein can be increased by mutating a gene or protein in the chosen host to be desensitized to the feedback inhibition. The phrase “desensitized to feedback inhibition” can include complete elimination of the feedback inhibition, and attenuation of the feedback inhibition, unless otherwise stated. Also, the phrase “being desensitized to feedback inhibition”, that is, when feedback inhibition is eliminated or attenuated, can also be referred to as “tolerant to feedback inhibition”. A protein showing an enhanced specific activity can be obtained by, for example, searching various organisms. Furthermore, a highly-active type of an existing protein may also be obtained by introducing a mutation into the existing protein. The mutation to be introduced may be, for example, substitution, deletion, insertion, and/or addition of one or several amino acid residues at one or several positions of the protein. The mutation can be introduced by, for example, such a site-specific mutation method as mentioned above. The mutation may also be introduced by, for example, a mutagenesis treatment. Examples of the mutagenesis treatment can include irradiation of X-ray, irradiation of ultraviolet, and a treatment with a mutation agent such as N-methyl-N′-nitro-N-nitrosoguanidine (MNNG), ethyl methanesulfonate (EMS), and methyl methanesulfonate (MMS). Furthermore, a random mutation may be induced by directly treating DNA in vitro with hydroxylamine. Enhancement of the specific activity may be independently used, or may be used in an arbitrary combination with such methods for enhancing gene expression as mentioned above.

The method for the transformation is not particularly limited, and conventionally known methods can be used. There can be used, for example, a method of treating recipient cells with calcium chloride so as to increase the permeability thereof for DNA, which has been reported for the Escherichia coli K-12 strain (Mandel, M. and Higa, A., J. Mol. Biol., 1970, 53, 159-162), and a method of preparing competent cells from cells which are in the growth phase, followed by transformation with DNA, which has been reported for Bacillus subtilis (Duncan, C. H., Wilson, G. A. and Young, F. E., Gene, 1977, 1:153-167). Alternatively, a method can be used of making DNA-recipient cells into protoplasts or spheroplasts, which can easily take up recombinant DNA, followed by introducing a recombinant DNA into the DNA-recipient cells, which is known to be applicable to Bacillus subtilis, actinomycetes, and yeasts (Chang, S. and Choen, S. N., 1979, Mol. Gen. Genet., 168:111-115; Bibb, M. J., Ward, J. M. and Hopwood, O. A., 1978, Nature, 274:398-400; Hinnen, A., Hicks, J. B. and Fink, G. R., 1978, Proc. Natl. Acad. Sci. USA, 75:1929-1933). Furthermore, the electric pulse method reported for coryneform bacteria (Japanese Patent Laid-open (Kokai) No. 2-207791) can also be used.

An increase in the activity of a protein can be confirmed by measuring the activity of the protein.

An increase in the activity of a protein can also be confirmed by confirming an increase in the expression of a gene encoding the protein. An increase in the expression of a gene can be confirmed by confirming an increase in the transcription amount of the gene, or by confirming an increase in the amount of a protein expressed from the gene.

An increase of the transcription amount of a gene can be confirmed by comparing the amount of mRNA transcribed from the gene with that of a non-modified strain such as a wild-type strain or parent strain. Examples of the method for evaluating the amount of mRNA can include Northern hybridization, RT-PCR, and so forth (Sambrook, J., et al., Molecular Cloning A Laboratory Manual/Third Edition, Cold Spring Harbor Laboratory Press, Cold Spring Harbor (USA), 2001). The amount of mRNA may be increased to, for example, 1.2 times or more, 1.5 times or more, 2 times or more, or 3 times or more of that of a non-modified strain.

An increase in the amount of a protein can be confirmed by Western blotting using antibodies (Molecular Cloning, Cold Spring Harbor Laboratory Press, Cold Spring Harbor (USA), 2001). The amount of the protein, such as the number of molecules of the protein per cell, may be increased to, for example, 1.2 times or more, 1.5 times or more, 2 times or more, or 3 times or more of that of a non-modified strain.

The aforementioned methods for increasing the activity of a protein can be applied to enhancement of the activities of arbitrary proteins such as an objective substance biosynthesis enzyme, phosphopantetheinylation enzyme, and uptake system of a substance, and enhancement of the expression of arbitrary genes such as genes encoding those arbitrary proteins.

### <1-4> Method for Reducing Activity of Protein

Hereafter, the methods for reducing the activity of a protein such as NCgl2048 protein will be explained.

The expression “the activity of a protein is reduced” means that the activity of the protein is reduced as compared with a non-modified strain. Specifically, the expression “the activity of a protein is reduced” may mean that the activity of the protein per cell is reduced as compared with that of a non-modified strain. The term “non-modified strain” can refer to a control strain that has not been modified so that the activity of an objective protein is reduced. Examples of the non-modified strain can include a wild-type strain and parent strain. Specific examples of the non-modified strain can include the respective type strains of the species of microorganisms. Specific examples of the non-modified strain can also include strains exemplified above in relation to the description of microorganisms. That is, in an embodiment, the activity of a protein may be reduced as compared with a type strain, i.e. the type strain of the species to which a microorganism belongs. In another embodiment, the activity of a protein may also be reduced as compared with the C. glutamicum ATCC 13869 strain. In another embodiment, the activity of a protein may also be reduced as compared with the C. glutamicum ATCC 13032 strain. In another embodiment, the activity of a protein may also be reduced as compared with the E. coli K-12 MG1655 strain. The phrase “the activity of a protein is reduced” can also include when the activity of the protein has completely disappeared. More specifically, the expression “the activity of a protein is reduced” may mean that the number of molecules of the protein per cell is reduced, and/or the function of each molecule of the protein is reduced as compared with those of a non-modified strain. That is, the term “activity” in the expression “the activity of a protein is reduced” is not limited to the catalytic activity of the protein, but may also mean the transcription amount of a gene, that is, the amount of mRNA, encoding the protein or the translation amount of the protein, that is, the amount of the protein. The phrase “the number of molecules of the protein per cell is reduced” can also include when the protein does not exist at all. The phrase “the function of each molecule of the protein is reduced” can also include when the function of each protein molecule has completely disappeared. The degree of the reduction in the activity of a protein is not particularly limited, so long as the activity is reduced as compared with that of a non-modified strain. The activity of a protein may be reduced to, for example, 50% or less, 20% or less, 10% or less, 5% or less, or 0% of that of a non-modified strain.

The modification for reducing the activity of a protein can be attained by, for example, reducing the expression of a gene encoding the protein. The phrase “the expression of a gene is reduced” means that the expression of the gene is reduced as compared with a non-modified strain, such as a wild-type strain and parent strain. Specifically, the phrase “the expression of a gene is reduced” may mean that the expression of the gene per cell is reduced as compared with that of a non-modified strain. More specifically, the phrase “the expression of a gene is reduced” may mean that the transcription amount of the gene, that is the amount of mRNA, is reduced, and/or the translation amount of the gene, that is, the amount of the protein expressed from the gene, is reduced. The phrase “the expression of a gene is reduced” can also include when the gene is not expressed at all. The phrase “the expression of a gene is reduced” can also be referred to as “the expression of a gene is attenuated”. The expression of a gene may be reduced to, for example, 50% or less, 20% or less, 10% or less, 5% or less, or 0% of that of a non-modified strain.

The reduction in gene expression may be due to, for example, a reduction in the transcription efficiency, a reduction in the translation efficiency, or a combination. The expression of a gene can be reduced by modifying an expression control sequence of the gene, such as a promoter, the Shine-Dalgarno (SD) sequence, which can also be referred to as ribosome-binding site (RBS), and a spacer region between RBS and the start codon of the gene. When an expression control sequence is modified, one or more nucleotides, two or more nucleotides, or three or more nucleotides, of the expression control sequence are modified. For example, the transcription efficiency of a gene can be reduced by, for example, replacing the promoter of the gene on a chromosome with a weaker promoter. The term “weaker promoter” can refer to a promoter providing an attenuated transcription of a gene compared with an inherent wild-type promoter of the gene. Examples of weaker promoters can include, for example, inducible promoters. That is, an inducible promoter may function as a weaker promoter under a non-induced condition, such as in the absence of the corresponding inducer. Examples of weaker promoters can also include, for example, P4 and P8 promoters (position 872-969 of SEQ ID NO: 82 and position 901-1046 of SEQ ID NO: 83, respectively). Furthermore, a part of or the entire expression control sequence may be deleted. The expression of a gene can also be reduced by, for example, manipulating a factor responsible for expression control. Examples of the factor responsible for expression control can include low molecules responsible for transcription or translation control, such as inducers, inhibitors, etc., proteins responsible for transcription or translation control, such as transcription factors etc., nucleic acids responsible for transcription or translation control, such as siRNA etc., and so forth. Furthermore, the expression of a gene can also be reduced by, for example, introducing a mutation that reduces the expression of the gene into the coding region of the gene. For example, the expression of a gene can be reduced by replacing a codon in the coding region of the gene with a synonymous codon used less frequently in a host. Furthermore, for example, the gene expression may be reduced due to disruption of a gene as described herein.

The modification for reducing the activity of a protein can also be attained by, for example, disrupting a gene encoding the protein. The phrase “a gene is disrupted” can mean that a gene is modified so that a protein that can normally function is not produced. The phrase “a protein that normally functions is not produced” can include when the protein is not produced at all from the gene, and when the protein of which the function, such as activity or property, per molecule is reduced or eliminated is produced from the gene.

Disruption of a gene can be attained by, for example, deleting the gene on a chromosome. The term “deletion of a gene” can refer to deletion of a partial or entire region of the coding region of the gene. Furthermore, the whole of a gene including sequences upstream and downstream from the coding region of the gene on a chromosome may be deleted. The region to be deleted may be any region, such as an N-terminal region (i.e. a region encoding an N-terminal region of a protein), an internal region, or a C-terminal region (i.e. a region encoding a C-terminal region of a protein), so long as the activity of the protein can be reduced. Deletion of a longer region will usually more surely inactivate the gene. The region to be deleted may be, for example, a region having a length of 10% or more, 20% or more, 30% or more, 40% or more, 50% or more, 60% or more, 70% or more, 80% or more, 90% or more, or 95% or more of the total length of the coding region of the gene. Furthermore, it is preferred that reading frames of the sequences upstream and downstream from the region to be deleted are not the same. Inconsistency of reading frames may cause a frameshift downstream of the region to be deleted.

Disruption of a gene can also be attained by, for example, introducing a mutation for an amino acid substitution (missense mutation), a stop codon (nonsense mutation), addition or deletion of one or two nucleotide residues (frame shift mutation), or the like into the coding region of the gene on a chromosome (Journal of Biological Chemistry, 272:8611-8617 (1997); Proceedings of the National Academy of Sciences, USA, 95 5511-5515 (1998); Journal of Biological Chemistry, 26 116, 20833-20839 (1991)).

Disruption of a gene can also be attained by, for example, inserting another nucleotide sequence into a coding region of the gene on a chromosome. Site of the insertion may be in any region of the gene, and insertion of a longer nucleotide sequence will usually more surely inactivate the gene. It is preferred that reading frames of the sequences upstream and downstream from the insertion site are not the same. Inconsistency of reading frames may cause a frameshift downstream of the region to be deleted. The other nucleotide sequence is not particularly limited so long as a sequence that reduces or eliminates the activity of the encoded protein is chosen, and examples thereof can include, for example, a marker gene such as antibiotic resistance genes, and a gene useful for production of an objective substance.

Particularly, disruption of a gene may be carried out so that the amino acid sequence of the encoded protein is deleted. In other words, the modification for reducing the activity of a protein can be attained by, for example, deleting the amino acid sequence of the protein, specifically, modifying a gene so as to encode a protein of which the amino acid sequence is deleted. The phrase “deletion of the amino acid sequence of a protein” can refer to deletion of a partial or entire region of the amino acid sequence of the protein. In addition, the phrase “deletion of the amino acid sequence of a protein” can mean that the original amino acid sequence disappears in the protein, and can also include when the original amino acid sequence is changed to another amino acid sequence. That is, for example, a region that was changed to another amino acid sequence by frameshift may be regarded as a deleted region. When the amino acid sequence of a protein is deleted, the total length of the protein is typically shortened, but there can also be cases where the total length of the protein is not changed or is extended. For example, by deletion of a partial or entire region of the coding region of a gene, a region encoded by the deleted region can be deleted in the encoded protein. In addition, for example, by introduction of a stop codon into the coding region of a gene, a region encoded by the downstream region of the introduction site can be deleted in the encoded protein. In addition, for example, by frameshift in the coding region of a gene, a region encoded by the frameshift region can be deleted in the encoded protein. The aforementioned descriptions concerning the position and length of the region to be deleted in deletion of a gene can be applied mutatis mutandis to the position and length of the region to be deleted in deletion of the amino acid sequence of a protein.

Such modification of a gene on a chromosome as described above can be attained by, for example, preparing a disruption-type gene modified so that it is unable to produce a protein that normally functions, and transforming a host with a recombinant DNA containing the disruption-type gene to cause homologous recombination between the disruption-type gene and the wild-type gene on a chromosome and thereby substitute the disruption-type gene for the wild-type gene on the chromosome. In this procedure, if a marker gene selected according to the characteristics of the host such as auxotrophy is included in the recombinant DNA, the operation becomes easier. Examples of the disruption-type gene can include a gene of which a partial or entire region of the coding region is deleted, a gene including a missense mutation, a gene including a nonsense mutation, a gene including a frame shift mutation, and a gene including insertion of a transposon or marker gene. The protein encoded by the disruption-type gene has a conformation different from that of the wild-type protein, even if it is produced, and thus the function thereof is reduced or eliminated. Such gene disruption based on gene substitution utilizing homologous recombination has already been established, and there are methods of using a linear DNA such as a method called “Red driven integration” (Datsenko, K. A, and Wanner, B. L., Proc. Natl. Acad. Sci. USA, 97:6640-6645 (2000)), and a method utilizing the Red driven integration in combination with an excision system derived from λ phage (Cho, E. H., Gumport, R. I., Gardner, J. F., J. Bacteriol., 184:5200-5203 (2002)) (refer to WO2005/010175), a method of using a plasmid having a temperature sensitive replication origin, a method of using a plasmid capable of conjugative transfer, a method of utilizing a suicide vector not having a replication origin that functions in a host (U.S. Pat. No. 6,303,383, Japanese Patent Laid-open (Kokai) No. 05-007491), and so forth.

Modification for reducing activity of a protein can also be attained by, for example, a mutagenesis treatment. Examples of the mutagenesis treatment can include irradiation of X-ray or ultraviolet and treatment with a mutation agent such as N-methyl-N′-nitro-N-nitrosoguanidine (MNNG), ethyl methanesulfonate (EMS), and methyl methanesulfonate (MMS).

Such methods for reducing the activity of a protein as mentioned above may be used independently or in an arbitrary combination.

When a protein functions as a complex made up of a plurality of subunits, a part or all of the plurality of subunits may be modified, so long as the activity of the protein is eventually reduced. That is, for example, a part or all of a plurality of genes that encode the respective subunits may be disrupted or the like. Furthermore, when there is a plurality of isozymes of a protein, a part or all of the activities of the plurality of isozymes may be reduced, so long as the activity of the protein is eventually reduced. That is, for example, a part or all of a plurality of genes that encode the respective isozymes may be disrupted or the like.

A reduction in the activity of a protein can be confirmed by measuring the activity of the protein.

A reduction in the activity of a protein can also be confirmed by confirming a reduction in the expression of a gene encoding the protein. A reduction in the expression of a gene can be confirmed by confirming a reduction in the transcription amount of the gene or a reduction in the amount of the protein expressed from the gene.

A reduction in the transcription amount of a gene can be confirmed by comparing the amount of mRNA transcribed from the gene with that observed in a non-modified strain. Examples of the method for evaluating the amount of mRNA can include Northern hybridization, RT-PCR, and so forth (Molecular Cloning, Cold Spring Harbor Laboratory Press, Cold Spring Harbor (USA), 2001). The amount of mRNA can be reduced to, for example, 50% or less, 20% or less, 10% or less, 5% or less, or 0%, of that of a non-modified strain.

A reduction in the amount of a protein can be confirmed by Western blotting using antibodies (Molecular Cloning, Cold Spring Harbor Laboratory Press, Cold Spring Harbor (USA) 2001). The amount of the protein, such as the number of molecules of the protein per cell, can be reduced to, for example, 50% or less, 20% or less, 10% or less, 5% or less, or 0%, of that of a non-modified strain.

Disruption of a gene can be confirmed by determining nucleotide sequence of a part or the whole of the gene, restriction enzyme map, full length, or the like of the gene depending on the means used for the disruption.

The aforementioned methods for reducing the activity of a protein can be applied to reduction in the activities of arbitrary proteins such as a byproduct generation enzyme, and reduction in the expression of arbitrary genes such as genes encoding those arbitrary proteins, besides attenuation of the activity of NCgl2048 protein.

### <2> Method for Producing Objective Substance

The method as described herein is a method for producing an objective substance by using the microorganism as described herein.

### <2-1> Fermentation Method

An objective substance can be produced by, for example, fermentation of the microorganism as described herein. That is, an embodiment of the method as described herein may be a method for producing an objective substance by fermentation of the microorganism. This embodiment can also be referred to as a “fermentation method”. Also, the step of producing an objective substance by fermentation of the microorganism as described herein can also be referred to as a “fermentation step”.

The fermentation step can be performed by cultivating the microorganism as described herein. Specifically, in the fermentation method, an objective substance can be produced from a carbon source. That is, the fermentation step may be, for example, a step of cultivating the microorganism in a culture medium, such as a culture medium containing a carbon source, to produce and accumulate the objective substance in the culture medium. That is, the fermentation method may be a method for producing an objective substance that comprises the step of cultivating the microorganism in a culture medium, such as a culture medium containing a carbon source, to produce and accumulate the objective substance in the culture medium. Also, in other words, the fermentation step may be, for example, a step of producing an objective substance from a carbon source by using the microorganism.

The culture medium to be used is not particularly limited, so long as the microorganism can proliferate in it and produce an objective substance. As the culture medium, for example, a typical culture medium used for culture of microorganisms such as bacteria and yeast can be used. The culture medium may contain carbon source, nitrogen source, phosphate source, and sulfur source, as well as other medium components such as various organic components and inorganic components as required. The types and concentrations of the medium components can be appropriately determined according to various conditions such as the type of the chosen microorganism.

The carbon source is not particularly limited, so long as the microorganism can utilize it and produce an objective substance. Specific examples of the carbon source can include, for example, saccharides such as glucose, fructose, sucrose, lactose, galactose, xylose, arabinose, blackstrap molasses, hydrolysates of starches, and hydrolysates of biomass; organic acids such as acetic acid, citric acid, succinic acid, and gluconic acid; alcohols such as ethanol, glycerol, and crude glycerol; and fatty acids. As the carbon source, in particular, plant-derived materials can be used. Examples of the plant can include, for example, corn, rice, wheat, soybean, sugarcane, beet, and cotton. Examples of the plant-derived materials can include, for example, organs such as root, stem, trunk, branch, leaf, flower, and seed, plant bodies including them, and decomposition products of these plant organs. The forms of the plant-derived materials at the time of use thereof are not particularly limited, and they can be used in any form such as unprocessed product, juice, ground product, and purified product. Pentoses such as xylose, hexoses such as glucose, or mixtures of them can be obtained from, for example, plant biomass, and used. Specifically, these saccharides can be obtained by subjecting a plant biomass to such a treatment as steam treatment, hydrolysis with concentrated acid, hydrolysis with diluted acid, hydrolysis with an enzyme such as cellulase, and alkaline treatment. Since hemicellulose is generally more easily hydrolyzed compared with cellulose, hemicellulose in a plant biomass may be hydrolyzed beforehand to liberate pentoses, and then cellulose may be hydrolyzed to generate hexoses. Furthermore, xylose may be supplied by conversion from hexoses by, for example, imparting a pathway for converting hexose such as glucose to xylose to the microorganism. As the carbon source, one carbon source may be used, or two or more carbon sources may be used in combination.

The concentration of the carbon source in the culture medium is not particularly limited, so long as the microorganism can proliferate and produce an objective substance. The concentration of the carbon source in the culture medium may be as high as possible within such a range that production of the objective substance is not inhibited. The initial concentration of the carbon source in the culture medium may be, for example, 5 to 30% (w/v), or 10 to 20% (w/v). Furthermore, the carbon source may be added to the culture medium as required. For example, the carbon source may be added to the culture medium in proportion to decrease or depletion of the carbon source accompanying progress of the fermentation. While the carbon source may be temporarily depleted so long as an objective substance can be eventually produced, it may be preferable to perform the culture so that the carbon source is not depleted or the carbon source does not continue to be depleted.

Specific examples of the nitrogen source can include, for example, ammonium salts such as ammonium sulfate, ammonium chloride, and ammonium phosphate, organic nitrogen sources such as peptone, yeast extract, meat extract, and soybean protein decomposition products, ammonia, and urea. Ammonia gas and aqueous ammonia used for pH adjustment may also be used as a nitrogen source. As the nitrogen source, one nitrogen source may be used, or two or more nitrogen sources may be used in combination.

Specific examples of the phosphate source can include, for example, phosphate salts such as potassium dihydrogenphosphate and dipotassium hydrogenphosphate, and phosphoric acid polymers such as pyrophosphoric acid. As the phosphate source, one phosphate source may be used, or two or more phosphate sources may be used in combination.

Specific examples of the sulfur source can include, for example, inorganic sulfur compounds such as sulfates, thiosulfates, and sulfites, and sulfur-containing amino acids such as cysteine, cystine, and glutathione. As the sulfur source, one sulfur source may be used, or two or more sulfur sources may be used in combination.

Specific examples of other various organic and inorganic components can include, for example, inorganic salts such as sodium chloride and potassium chloride; trace metals such as iron, manganese, magnesium, and calcium; vitamins such as vitamin B1, vitamin B2, vitamin B6, nicotinic acid, nicotinamide, and vitamin B12; amino acids; nucleic acids; and organic components containing these such as peptone, casamino acid, yeast extract, and soybean protein decomposition product. As the other various organic and inorganic components, one component may be used, or two or more components may be used in combination.

Furthermore, when an auxotrophic mutant strain that requires a nutrient such as amino acids for growth thereof is used, it is preferred that the culture medium contains such a required nutrient. Furthermore, the culture medium may contain a component used for production of an objective substance. Specific examples of such a component can include, for example, methyl group donors such as SAM and precursors thereof such as methionine.

Culture conditions are not particularly limited, so long as the microorganism can proliferate, and an objective substance is produced. The culture can be performed with, for example, typical conditions used for culture of microorganisms such as bacteria and yeast. The culture conditions may be appropriately determined according to various conditions such as the type of the chosen microorganism.

The culture can be performed by using a liquid medium. At the time of the culture, for example, the microorganism cultured on a solid medium such as agar medium may be directly inoculated into a liquid medium, or the microorganism cultured in a liquid medium as seed culture may be inoculated into a liquid medium for main culture. That is, the culture may be performed separately as seed culture and main culture. In such a case, the culture conditions of the seed culture and the main culture may be or may not be the same. It is sufficient that an objective substance is produced at least during the main culture. The amount of the microorganism present in the culture medium at the time of the start of the culture is not particularly limited. For example, a seed culture broth showing an OD660 of 4 to 100 may be inoculated to a culture medium for main culture in an amount of 0.1 to 100 mass %, or 1 to 50 mass %, at the time of the start of the culture.

The culture can be performed as batch culture, fed-batch culture, continuous culture, or a combination of these. The culture medium used at the start of the culture can also be referred to as a “starting medium”. The culture medium added to the culture system (e.g. fermentation tank) in the fed-batch culture or the continuous culture can also be referred to as a “feed medium”. To add a feed medium to the culture system in the fed-batch culture or the continuous culture can also be referred to as “feed”. Furthermore, when the culture is performed separately as seed culture and main culture, the culture schemes of the seed culture and the main culture may be or may not be the same. For example, both the seed culture and the main culture may be performed as batch culture. Alternatively, for example, the seed culture may be performed as batch culture, and the main culture may be performed as fed-batch culture or continuous culture.

The various components such as the carbon source may be present in the starting medium, feed medium, or both. That is, the various components such as the carbon source may be added to the culture medium independently or in an arbitrary combination during the culture. These components may be added once or a plurality of times, or may be continuously added. The types of the components present in the starting medium may be or may not be the same as the types of the components present in the feed medium. Furthermore, the concentrations of the components present in the starting medium may be or may not be the same as the concentrations of the components present in the feed medium. Furthermore, two or more kinds of feed media containing components of different types and/or different concentrations may be used. For example, when feeding is intermittently performed two or more times, the types and/or concentrations of components present in the feed medium may be or may not be the same for each feeding.

The culture can be performed, for example, under an aerobic condition. The term “aerobic condition” can refer to a condition where the dissolved oxygen concentration in the culture medium is 0.33 ppm or higher, or 1.5 ppm or higher. The oxygen concentration can be controlled to be, for example, 1 to 50%, or about 5%, of the saturated oxygen concentration. The culture can be performed, for example, with aeration or shaking. The pH of the culture medium may be, for example, 3 to 10, or 4.0 to 9.5. The pH of the culture medium can be adjusted during the culture as required. The pH of the culture medium can be adjusted by using various alkaline and acidic substances such as ammonia gas, aqueous ammonia, sodium carbonate, sodium bicarbonate, potassium carbonate, potassium bicarbonate, magnesium carbonate, sodium hydroxide, calcium hydroxide, and magnesium hydroxide. The culture temperature may be, for example, 20 to 45° C., or 25 to 37° C. The culture time may be, for example, 10 to 120 hours. The culture may be continued, for example, until the carbon source present in the culture medium is consumed, or until the activity of the microorganism is lost.

By cultivating the microorganism under such conditions as described above, an objective substance is accumulated in the culture medium.

Production of an objective substance can be confirmed by known methods used for detection or identification of compounds. Examples of such methods can include, for example, HPLC, UPLC, LC/MS, GC/MS, and NMR. These methods may be independently used, or may be used in an appropriate combination. These methods can also be used for determining the concentrations of various components present in the culture medium.

The produced objective substance can be appropriately collected. That is, the fermentation method may further comprise a step of collecting the objective substance. This step can also be referred to as a “collection step”. The collection step may be a step of collecting the objective substance from the culture broth, specifically from the culture medium. The objective substance can be collected by known methods used for separation and purification of compounds. Examples of such methods can include, for example, ion-exchange resin method, membrane treatment, precipitation, extraction, distillation, and crystallization. The objective substance can be collected specifically by extraction with an organic solvent such as ethyl acetate or by steam distillation. These methods may be independently used, or may be used in an appropriate combination.

Furthermore, when an objective substance precipitates in the culture medium, it can be collected by, for example, centrifugation or filtration. The objective substance precipitated in the culture medium and the objective substance dissolved in the culture medium may be isolated together after the objective substance dissolved in the culture medium is crystallized.

The collected objective substance may contain, for example, microbial cells, medium components, moisture, and by-product metabolites of the microorganism, in addition to the objective substance. Purity of the collected objective substance may be, for example, 30% (w/w) or higher, 50% (w/w) or higher, 70% (w/w) or higher, 80% (w/w) or higher, 90% (w/w) or higher, or 95% (w/w) or higher.

### <2-2> Bioconversion Method

An objective substance can also be produced by, for example, bioconversion using the microorganism as described herein. That is, another embodiment of the method as described herein may be a method for producing an objective substance by bioconversion using the microorganism. This embodiment can also be referred to as a “bioconversion method”. Also, the step of producing an objective substance by bioconversion using the microorganism can also be referred to as a “bioconversion step”.

Specifically, in the bioconversion method, an objective substance can be produced from a precursor of the objective substance. More specifically, in the bioconversion method, an objective substance can be produced by converting a precursor of the objective substance into the objective substance by using the microorganism. That is, the bioconversion step may be a step of converting a precursor of an objective substance into the objective substance by using the microorganism.

A precursor of an objective substance can also be referred to simply as a “precursor”. Examples of the precursor can include substances of which conversion into an object substance requires SAM. Specific examples of the precursor can include intermediates of the biosynthesis pathway of an object substance, such as those recited in relation to the descriptions of the objective substance biosynthesis enzymes, provided that conversion of the intermediates into the object substance requires SAM. More specific examples of the precursor can include, for example, protocatechuic acid, protocatechualdehyde, L-tryptophan, L-histidine, L-phenylalanine, L-tyrosine, L-arginine, L-ornithine, and glycine. Protocatechuic acid may be used as a precursor for producing, for example, vanillin, vanillic acid, or guaiacol. Protocatechualdehyde may be used as a precursor for producing, for example, vanillin. L-tryptophan may be used as a precursor for producing, for example, melatonin. L-histidine may be used as a precursor for producing, for example, ergothioneine. L-phenylalanine and L-tyrosine each may be used as a precursor for producing, for example, ferulic acid, 4-vinylguaiacol, or 4-ethylguaiacol. L-arginine and L-ornithine each may be used as a precursor for producing, for example, a polyamine. L-arginine and glycine each may be used as a precursor for producing, for example, creatine. As the precursor, one kind of precursor may be used, or two or more kinds of precursors may be used in combination. In cases where the precursor is a compound that can form a salt, the precursor may be used as a free compound, a salt thereof, or a mixture thereof. That is, the term “precursor” can refer to a precursor in a free form, a salt thereof, or a mixture thereof, unless otherwise stated. Examples of the salt can include, for example, sulfate salt, hydrochloride salt, carbonate salt, ammonium salt, sodium salt, and potassium salt. As the salt of the precursor, one kind of salt may be employed, or two or more kinds of salts may be employed in combination.

As the precursor, a commercial product may be used, or one appropriately prepared and obtained may be used. That is, the bioconversion method may further include a step of producing a precursor. The method for producing a precursor is not particularly limited, and for example, known methods can be used. A precursor can be produced by, for example, a chemical synthesis method, enzymatic method, bioconversion method, fermentation method, extraction method, or a combination of these. That is, for example, a precursor of an objective substance can be produced from a further precursor thereof using an enzyme that catalyzes the conversion of such a further precursor into the precursor of an objective substance, which enzyme can also be referred to as a “precursor biosynthesis enzyme”. Furthermore, for example, a precursor of an objective substance can be produced from a carbon source or such a further precursor by using a microorganism having a precursor-producing ability. The phrase “microorganism having a precursor-producing ability” can refer to a microorganism that is able to generate a precursor of an objective substance from a carbon source or a further precursor thereof. For example, examples of the method for producing protocatechuic acid according to an enzymatic method or bioconversion method can include the method of converting para-cresol into protocatechuic acid using Pseudomonas putida KS-0180 (Japanese Patent Laid-open (Kokai) No. 7-75589), the method of converting para-hydroxybenzoic acid into protocatechuic acid using an NADH-dependent para-hydroxybenzoic acid hydroxylase (Japanese Patent Laid-open (Kokai) No. 5-244941), the method of producing protocatechuic acid by cultivating a transformant harboring a gene that is involved in the reaction of generating protocatechuic acid from terephthalic acid in a culture medium containing terephthalic acid (Japanese Patent Laid-open (Kokai) No. 2007-104942), and the method of producing protocatechuic acid from a precursor thereof by using a microorganism having protocatechuic acid-producing ability and having a reduced activity of protocatechuic acid 5-oxidase or being deficient in that activity (Japanese Patent Laid-open (Kokai) No. 2010-207094). Furthermore, examples of the method for producing protocatechuic acid by fermentation can include the method of producing protocatechuic acid by using a bacterium of the genus Brevibacterium and acetic acid as a carbon source (Japanese Patent Laid-open (Kokai) No. 50-89592), the method of producing protocatechuic acid by using a bacterium of the genus Escherichia or Klebsiella into which a gene encoding 3-dihydroshikimate dehydrogenase has been introduced, and glucose as a carbon source (U.S. Pat. No. 5,272,073). Furthermore, protocatechualdehyde can be produced by using protocatechuic acid as a precursor according to an enzymatic method using ACAR or a bioconversion method using a microorganism having ACAR. The produced precursor can be used for the bioconversion method as it is, or after being subjected to an appropriate treatment such as concentration, dilution, drying, dissolution, fractionation, extraction, and purification, as required. That is, as the precursor, for example, a purified product purified to a desired extent may be used, or a material containing a precursor may be used. The material containing a precursor is not particularly limited so long as the microorganism can use the precursor. Specific examples of the material containing a precursor can include a culture broth obtained by cultivating a microorganism having a precursor-producing ability, a culture supernatant separated from the culture broth, and processed products thereof such as concentrated products, such as concentrated liquid, thereof and dried products thereof.

In an embodiment, the bioconversion step can be performed by, for example, cultivating the microorganism as described herein. This embodiment can also be referred to as a “first embodiment of the bioconversion method”. That is, the bioconversion step may be, for example, a step of cultivating the microorganism in a culture medium containing a precursor of an objective substance to convert the precursor into the objective substance. The bioconversion step may be, specifically, a step of cultivating the microorganism in a culture medium containing a precursor of an objective substance to produce and accumulate the objective substance in the culture medium.

The culture medium to be used is not particularly limited, so long as the culture medium contains a precursor of an objective substance, and the microorganism can proliferate in it and produce the objective substance. Culture conditions are not particularly limited, so long as the microorganism can proliferate, and an objective substance is produced. The descriptions concerning the culture mentioned for the fermentation method, such as those concerning the culture medium and culture conditions, can be applied mutatis mutandis to the culture in the first embodiment of the bioconversion method, except that the culture medium contains the precursor in the first embodiment.

The precursor may be present in the culture medium over the whole period of the culture, or may be present in the culture medium during only a partial period of the culture. That is, the phrase “cultivating a microorganism in a culture medium containing a precursor” does not necessarily mean that the precursor is present in the culture medium over the whole period of the culture. For example, the precursor may be or may not be present in the culture medium from the start of the culture. When the precursor is not present in the culture medium at the time of the start of the culture, the precursor is added to the culture medium after the start of the culture. Timing of the addition can be appropriately determined according to various conditions such as the length of the culture period. For example, after the microorganism sufficiently grows, the precursor may be added to the culture medium. Furthermore, in any case, the precursor may be added to the culture medium as required. For example, the precursor may be added to the culture medium in proportion to decrease or depletion of the precursor accompanying generation of an objective substance. Methods for adding the precursor to the culture medium are not particularly limited. For example, the precursor can be added to the culture medium by feeding a feed medium containing the precursor to the culture medium. Furthermore, for example, the microorganism as described herein and a microorganism having a precursor-producing ability can be co-cultured to allow the microorganism having a precursor-producing ability to produce the precursor in the culture medium, and thereby add the precursor to the culture medium. These methods of addition may be independently used, or may be used in an appropriate combination. The concentration of the precursor in the culture medium is not particularly limited so long as the microorganism can use the precursor as a raw material of an objective substance. The concentration of the precursor in the culture medium, for example, may be 0.1 g/L or higher, 1 g/L or higher, 2 g/L or higher, 5 g/L or higher, 10 g/L or higher, or 15 g/L or higher, or may be 200 g/L or lower, 100 g/L or lower, 50 g/L or lower, or 20 g/L or lower, or may be within a range defined with a combination thereof, in terms of the weight of the free compound. The precursor may or may not be present in the culture medium at a concentration within the range exemplified above over the whole period of the culture. For example, the precursor may be present in the culture medium at a concentration within the range exemplified above at the time of the start of the culture, or it may be added to the culture medium so that a concentration within the range exemplified above is attained after the start of the culture. In cases where the culture is performed separately as seed culture and main culture, it is sufficient that an objective substance is produced at least during the main culture. Hence, it is sufficient that the precursor is present in the culture medium at least during the main culture, that is, over the whole period of the main culture or during a partial period of the main culture, and that is, the precursor may be or may not be present in the culture medium during the seed culture. In such cases, terms regarding the culture, such as “culture period (period of culture)” and “start of culture”, can be read as those regarding the main culture.

In another embodiment, the bioconversion step can also be performed by, for example, using cells of the microorganism as described herein. This embodiment can also be referred to as a “second embodiment of the bioconversion method”. That is, the bioconversion step may be, for example, a step of converting a precursor of an objective substance in a reaction mixture into the objective substance by using cells of the microorganism. The bioconversion step may be, specifically, a step of allowing cells of the microorganism to act on a precursor of an objective substance in a reaction mixture to generate and accumulate the objective substance in the reaction mixture. The bioconversion step performed by using such cells can also be referred to as a “conversion reaction”.

Cells of the microorganism can be obtained by cultivating the microorganism. The culture method for obtaining the cells is not particularly limited so long as the microorganism can proliferate. At the time of the culture for obtaining the cells, the precursor may or may not be present in the culture medium. Also, at the time of the culture for obtaining the cells, an objective substance may or may not be produced in the culture medium. The descriptions concerning the culture mentioned for the fermentation method, such as those concerning the culture medium and culture conditions, can be applied mutatis mutandis to the culture for obtaining the cells used for the second embodiment of the bioconversion method.

The cells may be used for the conversion reaction while being present in the culture broth (specifically, culture medium), or after being collected from the culture broth (specifically, culture medium). The cells may also be used for the conversion reaction after being subjected to a treatment as required. That is, examples of the cells can include a culture broth containing the cells, the cells collected from the culture broth, and a processed product thereof. In other words, examples of the cells can include cells present in a culture broth of the microorganism, cells collected from the culture broth, or cells present in a processed product thereof. Examples of the processed product can include products obtained by subjecting the cells to a treatment, specifically by subjecting a culture broth containing the cells, or the cells collected from the culture broth to a treatment. Cells in these forms may be independently used, or may be used in an appropriate combination.

The method for collecting the cells from the culture medium is not particularly limited, and for example, known methods can be used. Examples of such methods can include, for example, spontaneous precipitation, centrifugation, and filtration. A flocculant may also be used. These methods may be independently used, or may be used in an appropriate combination. The collected cells can be washed as required by using an appropriate medium. The collected cells can be re-suspended as required by using an appropriate medium. Examples of the medium usable for washing or suspending the cells can include, for example, aqueous media (aqueous solvents) such as water and aqueous buffer.

Examples of the treatment of the cells can include, for example, dilution, condensation, immobilization on a carrier such as acrylamide and carrageenan, freezing and thawing treatment, and treatment for increasing permeability of cell membranes. Permeability of cell membranes can be increased by, for example, using a surfactant or organic solvent. These treatments may be independently used, or may be used in an appropriate combination.

The cells used for the conversion reaction are not particularly limited so long as the cells have the objective substance-producing ability. It is preferred that the cells maintain their metabolic activities. The phrase “the cells maintain their metabolic activities” may mean that the cells have an ability to utilize a carbon source to generate or regenerate a substance required for producing an objective substance. Examples of such a substance can include, for example, ATP, electron donors such as NADH and NADPH, and methyl group donors such as SAM. The cells may have or may not have proliferation ability.

The conversion reaction can be carried out in an appropriate reaction mixture. Specifically, the conversion reaction can be carried out by allowing the cells and the precursor to coexist in an appropriate reaction mixture. The conversion reaction may be carried out by the batch method or may be carried out by the column method. In the case of the batch method, the conversion reaction can be carried out by, for example, mixing the cells of the microorganism and the precursor in a reaction mixture contained in a reaction vessel. The conversion reaction may be carried out statically, or may be carried out with stirring or shaking the reaction mixture. In the case of the column method, the conversion reaction can be carried out by, for example, passing a reaction mixture containing the precursor through a column filled with immobilized cells. Examples of the reaction mixture can include those based on an aqueous medium (aqueous solvent) such as water and aqueous buffer.

The reaction mixture may contain components other than the precursor as required, in addition to the precursor. Examples of the components other than the precursor can include ATP, electron donors such as NADH and NADPH, methyl group donors such as SAM, metal ions, buffering agents, surfactants, organic solvents, carbon sources, phosphate sources, and other various medium components. That is, for example, a culture medium containing the precursor may also be used as a reaction mixture. That is, the descriptions concerning the culture medium mentioned for the first embodiment of the bioconversion method may also be applied mutatis mutandis to the reaction mixture in the second embodiment of the bioconversion method. The types and concentrations of the components present in the reaction mixture may be determined according to various conditions such as the type of the precursor to be used and the form of the cells to be used.

Conditions of the conversion reaction, such as dissolved oxygen concentration, pH of the reaction mixture, reaction temperature, reaction time, concentrations of various components, etc., are not particularly limited so long as an objective substance is generated. The conversion reaction can be performed with, for example, typical conditions used for substance conversion using microbial cells such as resting cells. The conditions of the conversion reaction may be determined according to various conditions such as the type of chosen microorganism. The conversion reaction can be performed, for example, under an aerobic condition. The term “aerobic condition” can refer to a condition where the dissolved oxygen concentration in the reaction mixture is 0.33 ppm or higher, or 1.5 ppm or higher. The oxygen concentration can be controlled to be, for example, 1 to 50%, or about 5%, of the saturated oxygen concentration. The pH of the reaction mixture may be, for example, usually 6.0 to 10.0, or 6.5 to 9.0. The reaction temperature may be, for example, 15 to 50° C., 15 to 45° C., or 20 to 40° C. The reaction time may be, for example, 5 minutes to 200 hours. In the case of the column method, the loading rate of the reaction mixture may be, for example, such a rate that the reaction time falls within the range of the reaction time exemplified above. Furthermore, the conversion reaction can also be performed with, for example, a culture condition, such as typical conditions used for culture of microorganisms such as bacteria and yeast. During the conversion reaction, the cells may or may not proliferate. That is, the descriptions concerning the culture conditions for the first embodiment of the bioconversion method may also be applied mutatis mutandis to the conditions of the conversion reaction in the second embodiment of the bioconversion method, except that the cells may or may not proliferate in the second embodiment. In such a case, the culture conditions for obtaining the cells and the conditions of the conversion reaction may be the same or different. The concentration of the precursor in the reaction mixture, for example, may be 0.1 g/L or higher, 1 g/L or higher, 2 g/L or higher, 5 g/L or higher, 10 g/L or higher, or 15 g/L or higher, or may be 200 g/L or lower, 100 g/L or lower, 50 g/L or lower, or 20 g/L or lower, or may be within a range defined with a combination thereof, in terms of the weight of the free compound. The density of the cells in the reaction mixture, for example, may be 1 or higher, or may be 300 or lower, or may be within a range defined with a combination thereof, in terms of the optical density (OD) at 600 nm.

During the conversion reaction, the cells, the precursor, and the other components may be added to the reaction mixture independently or in any arbitrary combination thereof. For example, the precursor may be added to the reaction mixture in proportion to decrease or depletion of the precursor accompanying generation of an objective substance. These components may be added once or a plurality of times, or may be continuously added.

Methods for adding the various components such as the precursor to the reaction mixture are not particularly limited. These components each can be added to the reaction mixture by, for example, directly adding them to the reaction mixture. Furthermore, for example, the microorganism as described herein and a microorganism having a precursor-producing ability can be co-cultured to allow the microorganism having a precursor-producing ability to produce the precursor in the reaction mixture, and thereby supply the precursor to the reaction mixture. Furthermore, for example, components such as ATP, electron donors, and methyl group donors each may be generated or regenerated in the reaction mixture, may be generated or regenerated in the cells of the microorganism, or may be generated or regenerated by a coupling reaction between different cells. For example, when cells of the microorganism maintain the metabolic activities thereof, they can generate or regenerate components such as ATP, electron donors, and methyl group donors within them by using a carbon source. For example, specifically, the microorganism may have an enhanced ability for generating or regenerating SAM, and the generated or regenerated SAM by it may be used for the conversion reaction. The generation or regeneration of SAM may further be enhanced in combination with any other method for generating or regenerating SAM. In addition, examples of the method for generating or regenerating ATP can include, for example, the method of supplying ATP from a carbon source by using a Corynebacterium bacterium (Hori, H. et al., Appl. Microbiol. Biotechnol., 48(6):693-698 (1997)), the method of regenerating ATP by using yeast cells and glucose (Yamamoto, S et al., Biosci. Biotechnol. Biochem., 69(4):784-789 (2005)), the method of regenerating ATP using phosphoenolpyruvic acid and pyruvate kinase (C. Aug'e and Ch. Gautheron, Tetrahedron Lett., 29:789-790 (1988)), and the method of regenerating ATP by using polyphosphoric acid and polyphosphate kinase (Murata, K. et al., Agric. Biol. Chem., 52(6):1471-1477 (1988)).

Furthermore, the reaction conditions may be constant from the start to the end of the conversion reaction, or they may vary during the conversion reaction. The expression “the reaction conditions vary during the conversion reaction” can include not only when the reaction conditions are temporally changed, but also includes when the reaction conditions are spatially changed. The expression “the reaction conditions are spatially changed” means that, for example, when the conversion reaction is performed by the column method, the reaction conditions such as reaction temperature and cell density differ depending on position in the flow.

A culture broth (specifically, culture medium) or reaction mixture containing an objective substance is obtained by carrying out the bioconversion step as described above. Confirmation of the production of the objective substance and collection of the objective substance can be carried out in the same manners as those for the fermentation method described above. That is, the bioconversion method may further comprise the collection step, such as a step of collecting the objective substance from the culture broth (specifically, culture medium) or reaction mixture. The collected objective substance may contain, for example, microbial cells, medium components, reaction mixture components, moisture, and by-product metabolites of the microorganism, in addition to the objective substance. Purity of the collected objective substance may be, for example, 30% (w/w) or higher, 50% (w/w) or higher, 70% (w/w) or higher, 80% (w/w) or higher, 90% (w/w) or higher, or 95% (w/w) or higher.

<2-3> Method for Producing Vanillin and other Objective Substances

When an objective substance is produced by using the microorganism as described herein, that is, by the fermentation method or bioconversion method, the thus-produced objective substance can further be converted to another objective substance. The present invention thus provides a method for producing a second objective substance, that is objective substance B, comprising steps of producing a first objective substance, that is objective substance A, by using the microorganism, that is, by the fermentation method or bioconversion method, and converting the thus-produced first objective substance A to the second objective substance B.

For example, when vanillic acid is produced by using the microorganism as described herein, that is, by the fermentation method or bioconversion method, the thus-produced vanillic acid can further be converted to vanillin. The present invention thus provides a method for producing vanillin comprising steps of producing vanillic acid by using the microorganism, that is, by the fermentation method or bioconversion method, and converting thus-produced vanillic acid into vanillin. This method can also be referred to as a “vanillin production method”.

Vanillic acid produced by using the microorganism can be used for the conversion into vanillin as it is, or after being subjected to an appropriate treatment such as concentration, dilution, drying, dissolution, fractionation, extraction, and purification, as required. That is, as vanillic acid, for example, a purified product purified to a desired extent may be used, or a material containing vanillic acid may be used. The material containing vanillic acid is not particularly limited so long as a component that catalyzes the conversion, such as a microorganism and an enzyme, can use vanillic acid. Specific examples of the material containing vanillic acid can include a culture broth or reaction mixture containing vanillic acid, a supernatant separated from the culture broth or reaction mixture, and processed products thereof such as concentrated products, such as concentrated liquid, thereof and dried products thereof.

The method for converting vanillic acid into vanillin is not particularly limited.

Vanillic acid can be converted into vanillin by, for example, a bioconversion method using a microorganism having ACAR. The microorganism having ACAR may be or may not be modified so that the activity of NCgl2048 protein is reduced. The descriptions concerning the microorganism as described herein can be applied mutatis mutandis to the microorganism having ACAR, except that the microorganism having ACAR and may be or may not be modified so that the activity of NCgl2048 protein is reduced. The microorganism having ACAR may be modified so that the activity or activities of one or more of ACAR, PPT, and the vanillic acid uptake system is/are enhanced. In addition, the descriptions concerning the bioconversion method for producing an objective substance using the microorganism can be applied mutatis mutandis to the bioconversion method for converting vanillic acid into vanillin using a microorganism having ACAR.

Vanillic acid can also be converted into vanillin by, for example, an enzymatic method using ACAR.

ACAR can be produced by allowing a host having an ACAR gene to express the ACAR gene. ACAR can also be produced with a cell-free protein expression system.

A host having an ACAR gene can also be referred to as a “host having ACAR”. The host having an ACAR gene may be a host inherently having the ACAR gene or may be a host modified to have the ACAR gene. Examples of the host inherently having an ACAR gene can include organisms from which ACARs exemplified above are derived. Examples of the host modified to have an ACAR gene can include hosts into which the ACAR gene has been introduced. Also, a host inherently having an ACAR gene may be modified so that the ACAR is increased. The host to be used for expression of ACAR is not particularly limited, so long as the host can express an ACAR that can function. Examples of the host can include, for example, microorganisms such as bacteria and yeast (fungi), plant cells, insect cells, and animal cells.

An ACAR gene can be expressed by cultivating a host having the ACAR gene. The culture method is not particularly limited so long as the host having the ACAR gene can proliferate and express ACAR. The descriptions concerning the culture for the fermentation method can be applied mutatis mutandis to the culture of the host having the ACAR gene. As necessarily, expression of the ACAR gene can be induced. As a result of cultivation, a culture broth containing ACAR can be obtained. ACAR can be accumulated in cells of the host and/or the culture medium.

ACAR contained in the cells of the host, the culture medium, or the like may be used as they are for the enzymatic reaction, or ACAR purified therefrom may be used for the enzymatic reaction. Purification can be performed to a desired extent. That is, as ACAR, purified ACAR may be used, or a fraction containing ACAR may be used. Such a fraction is not particularly limited, so long as ACAR contained therein can act to vanillic acid. Examples of such a fraction can include, a culture broth of a host having an ACAR gene, that is, a host having ACAR; cells collected from the culture broth; processed products of the cells, such as cell disruptant, cell lysate, cell extract, and immobilized cells such as those immobilized with acrylamide, carrageenan, or the like; a culture supernatant collected from the culture broth; partially purified products thereof, such as a crude product; and combinations thereof. These fractions may be used independently, or in combination with purified ACAR.

The enzymatic reaction can be performed by allowing ACAR to act on vanillic acid. Conditions of the enzymatic reaction are not particularly limited so long as vanillin is generated. The enzymatic reaction can be performed with, for example, typical conditions used for substance conversion using an enzyme or microbial cells such as resting cells. For example, the descriptions concerning the conversion reaction in in the second embodiment of the bioconversion method may also be applied mutatis mutandis to the enzymatic reaction in the vanillin production method.

A reaction mixture containing vanillin is obtained by carrying out the conversion as described above. Confirmation of the production of vanillin and collection of vanillin can be carried out in the same manners as those for the fermentation method described above. That is, the vanillin production method may further comprise a step of collecting vanillin from the reaction mixture. The collected vanillin may contain, for example, microbial cells, medium components, reaction mixture components, ACAR, moisture, and by-product metabolites of the microorganism, in addition to vanillin. Purity of the collected vanillin may be, for example, 30% (w/w) or higher, 50% (w/w) or higher, 70% (w/w) or higher, 80% (w/w) or higher, 90% (w/w) or higher, or 95% (w/w) or higher.

Vanillic acid can also be converted to guaiacol by, for example, a bioconversion method using a microorganism having VDC or an enzymatic method using VDC. Ferulic acid can be converted to 4-vinylguaiacol by, for example, a bioconversion method using a microorganism having FDC or an enzymatic method using FDC. 4-vinylguaiacol can be converted to 4-ethylguaiacol by, for example, a bioconversion method using a microorganism having VPR or an enzymatic method using VPR. Ferulic acid can also be converted to 4-ethylguaiacol by a combination of these methods. Specifically, ferulic acid can be converted to 4-ethylguaiacol by, for example, using FDC or a microorganism having FDC in combination with VPR or a microorganism having VPR simultaneously or sequentially, or using a microorganism having both FDC and VPR. The aforementioned descriptions concerning the vanillin production method can be applied mutatis mutandis to methods for producing other objective substances.

### Examples

Hereafter, the present invention will be more specifically explained with reference to the following non-limiting examples.

In this example, a strain having an attenuated expression of NCgl2048 gene was constructed from the Corynebacterium glutamicum 2256 strain (ATCC 13869) as a parent strain, and vanillic acid production was performed with the constructed strain.

### <1> Construction of Strain Deficient in Vanillate Demethylase Genes (FKS0165 Strain)

It has been reported that, in coryneform bacteria, vanillin is metabolized in the order of vanillin−>vanillic acid−> protocatechuic acid, and utilized (Current Microbiology, 2005, Vol. 51, pp. 59-65). The conversion reaction from vanillic acid to protocatechuic acid is catalyzed by vanillate demethylase. The vanA gene and vanB gene encode the subunit A and subunit B of vanillate demethylase, respectively. The vanK gene encodes the vanillic acid uptake system, and constitutes the vanABK operon together with the vanAB genes (M. T. Chaudhry, et al., Microbiology, 2007, 153:857-865). Therefore, a strain deficient in utilization ability of an objective substance such as vanillin and vanillic acid (FKS0165 strain) was first constructed from C. glutamicum 2256 strain by deleting the vanABK operon. The procedure is shown below.

<1-1> Construction of Plasmid pBS4SΔvanABK56 for Deletion of vanABK Genes

PCR was performed by using the genomic DNA of the C. glutamicum 2256 strain as the template, and the synthetic DNAs of SEQ ID NOS: 51 and 52 as the primers to obtain a PCR product containing an N-terminus side coding region of the vanA gene. Separately, PCR was also performed by using the genomic DNA of the C. glutamicum 2256 strain as the template, and the synthetic DNAs of SEQ ID NOS: 53 and 54 as the primers to obtain a PCR product containing a C-terminus side coding region of the vanK gene. The sequences of SEQ ID NOS: 52 and 53 are partially complementary to each other. Then, the PCR product containing the N-terminus side coding region of the vanA gene and the PCR product containing the C-terminus side coding region of the vanK gene were mixed in approximately equimolar amounts, and inserted into the pBS4S vector (WO2007/046389) treated with BamHI and PstI by using In Fusion HD Cloning Kit (Clontech). With this DNA, competent cells of Escherichia coli JM109 (Takara Bio) were transformed, and the cells were applied to the LB medium containing 100 μM IPTG, 40 μg/mL of X-Gal, and 40 μg/mL of kanamycin, and cultured overnight. Then, white colonies that appeared were picked up, and separated into single colonies to obtain transformants. Plasmids were extracted from the obtained transformants, and one into which the target PCR product was inserted was designated as pBS4SΔvanABK56.

### <1-2> Construction of FKS0165 Strain

pBS4SΔvanABK56 obtained above does not contain the region enabling autonomous replication of the plasmid in cells of coryneform bacteria. Therefore, if coryneform bacteria are transformed with this plasmid, a strain in which this plasmid is incorporated into the genome by homologous recombination appears as a transformant, although it occurs at an extremely low frequency. Therefore, pBS4SΔvanABK56 was introduced into the C. glutamicum 2256 strain by the electric pulse method. The cells were applied to the CM-Dex agar medium (5 g/L of glucose, 10 g/L of polypeptone, 10 g/L of yeast extract, 1 g/L of KH₂PO₄, 0.4 g/L of MgSO₄-7H₂O, 0.01 g/L of FeSO₄-7H₂O, 0.01 g/L of MnSO₄-7H₂O, 3 g/L of urea, 1.2 g/L of soybean hydrolysate, 10 μg/L of biotin, 15 g/L of agar, adjusted to pH 7.5 with NaOH) containing 25 μg/mL of kanamycin, and cultured at 31.5° C. It was confirmed by PCR that the grown strain was a once-recombinant strain in which pBS4SΔvanABK56 was incorporated into the genome by homologous recombination. This once-recombinant strain had both the wild-type vanABK genes, and the deficient-type vanABK genes.

The once-recombinant strain was cultured overnight in the CM-Dex liquid medium (having the same composition as that of the CM-Dex agar medium except that it does not contain agar), and the culture broth was applied to the S10 agar medium (100 g/L of sucrose, 10 g/L of polypeptone, 10 g/L of yeast extract, 1 g/L of KH₂PO₄, 0.4 g/L of MgSO₄-7H₂O, 0.01 g/L of FeSO₄-7H₂O, 0.01 g/L of MnSO₄-4-5H₂O, 3 g/L of urea, 1.2 g/L of soybean protein hydrolysate solution, 10 μg/L of biotin, 20 g/L of agar, adjusted to pH 7.5 with NaOH, and autoclaved at 120° C. for 20 minutes), and cultured at 31.5° C. Among the colonies that appeared, a strain that showed kanamycin susceptibility was purified on the CM-Dex agar medium. By preparing genomic DNA from the purified strain, and using it to perform PCR with the synthetic DNAs of SEQ ID NOS: 55 and 56 as the primers, deletion of the vanABK genes was confirmed, and the strain was designated as FKS0165 strain.

<2> Construction of Strain Deficient in Alcohol Dehydrogenase Homologue genes (FKFC14 strain)

Subsequently, by using the Corynebacterium glutamicum FKS0165 strain as a parent strain, there was constructed a strain FKFC14, which is deficient in alcohol dehydrogenase homologue genes, i.e. NCgl0324 gene (adhC), NCgl0313 gene (adhE), and NCgl2709 gene (adhA), via the following procedure.

### <2-1> Construction of FKFCS Strain (FKS0165ΔNCgl0324 Strain)

<2-1-1> Construction of Plasmid pBS4SΔ2256adhC for Deletion of NCgl0324 Gene

PCR was performed by using the genomic DNA of the C. glutamicum 2256 strain as the template, and the synthetic DNAs of SEQ ID NOS: 57 and 58 as the primers to obtain a PCR product containing an N-terminus side coding region of the NCgl0324 gene. Separately, PCR was performed by using the genomic DNA of the C. glutamicum 2256 strain as the template, and the synthetic DNAs of SEQ ID NOS: 59 and 60 as the primers to obtain a PCR product containing a C-terminus side coding region of the NCgl0324 gene. The sequences of SEQ ID NOS: 58 and 59 are partially complementary to each other. Then, approximately equimolar amounts of the PCR product containing the N-terminus side coding region of the NCgl0324 gene and the PCR product containing the C-terminus side coding region of the NCgl0324 gene were mixed, and inserted into the pBS4S vector (WO2007/046389) treated with BamHI and PstI by using In Fusion HD Cloning Kit (Clontech). With this DNA, competent cells of Escherichia coli JM109 (Takara Bio) were transformed, and the cells were applied to the LB medium containing 100 μM IPTG, 40 μg/mL of X-Gal, and 40 μg/mL of kanamycin, and cultured overnight. Then, white colonies that appeared were picked up, and separated into single colonies to obtain transformants. Plasmids were extracted from the obtained transformants, and one in which the target PCR product was inserted was designated as pBS4SΔ2256adhC.

### <2-1-2> Construction of FKFCS Strain (FKS0165ΔNCgl0324 Strain)

Since pBS4SΔ2256adhC obtained above does not contain the region enabling autonomous replication of the plasmid in cells of coryneform bacteria, if coryneform bacteria are transformed with this plasmid, a strain in which this plasmid is incorporated into the genome by homologous recombination appears as a transformant, although it occurs at an extremely low frequency. Therefore, pBS4SΔ2256adhC was introduced into the C. glutamicum FKS0165 strain by the electric pulse method. The cells were applied to the CM-Dex agar medium containing 25 μg/mL of kanamycin, and cultured at 31.5° C. It was confirmed by PCR that the grown strain was a once-recombinant strain in which pBS4SΔ2256adhC was incorporated into the genome by homologous recombination. This once-recombinant strain had both the wild-type NCgl0324 gene, and the deficient-type NCgl0324 gene.

The once-recombinant strain was cultured overnight in the CM-Dex liquid medium, the culture broth was applied to the S10 agar medium, and culture was performed at 31.5° C. Among the colonies that appeared, a strain that showed kanamycin susceptibility was purified on the CM-Dex agar medium. Genomic DNA was prepared from the purified strain, and used to perform PCR with the synthetic DNAs of SEQ ID NOS: 61 and 62 as the primers to confirm deletion of the NCgl0324 gene, and the strain was designated as FKFCS strain.

<2-2> Construction of FKFC11 Strain (2256ΔvanABKΔNCgl0324ΔNCgl0313 Strain)
<2-2-1> Construction of Plasmid pBS4SΔ2256adhE for Deletion of NCgl0313 Gene

PCR was performed by using the genomic DNA of the C. glutamicum 2256 strain as the template, and the synthetic DNAs of SEQ ID NOS: 63 and 64 as the primers to obtain a PCR product containing an N-terminus side coding region of the NCgl0313 gene. Separately, PCR was performed by using the genomic DNA of the C. glutamicum 2256 strain as the template, and the synthetic DNAs of SEQ ID NOS: 65 and 66 as the primers to obtain a PCR product containing a C-terminus side coding region of the NCgl0313 gene. The sequences of SEQ ID NOS: 64 and 65 are partially complementary to each other. Then, approximately equimolar amounts of the PCR product containing the N-terminus side coding region of the NCgl0313 gene and the PCR product containing the C-terminus side coding region of the NCgl0313 gene were mixed, and inserted into the pBS4S vector (WO2007/046389) treated with BamHI and PstI by using In Fusion HD Cloning Kit (Clontech). With this DNA, competent cells of Escherichia coli JM109 (Takara Bio) were transformed, and the cells were applied to the LB medium containing 100 μM IPTG, 40 μg/mL of X-Gal, and 40 μg/mL of kanamycin, and cultured overnight. Then, white colonies that appeared were picked up, and separated into single colonies to obtain transformants. Plasmids were extracted from the obtained transformants, and one in which the target PCR product was inserted was designated as pBS4SΔ2256adhE.

<2-2-2> Construction of FKFC11 Strain (2256ΔvanABKΔNCgl0324ΔNCgl0313 Strain)

Since pBS4SΔ2256adhE obtained above does not contain the region enabling autonomous replication of the plasmid in cells of coryneform bacteria, if coryneform bacteria are transformed with this plasmid, a strain in which this plasmid is incorporated into the genome by homologous recombination appears as a transformant, although it occurs at an extremely low frequency. Therefore, pBS4SΔ2256adhE was introduced into the C. glutamicum FKFC5 strain by the electric pulse method. The cells were applied to the CM-Dex agar medium containing 25 μg/mL of kanamycin, and cultured at 31.5° C. It was confirmed by PCR that the grown strain was a once-recombinant strain in which pBS4SΔ2256adhE was incorporated into the genome by homologous recombination. This once-recombinant strain had both the wild-type NCgl0313 gene, and the deficient-type NCgl0313 gene.

The once-recombinant strain was cultured overnight in the CM-Dex liquid medium, the culture broth was applied to the S10 agar medium, and culture was performed at 31.5° C. Among the colonies that appeared, a strain that showed kanamycin susceptibility was purified on the CM-Dex agar medium. Genomic DNA was prepared from the purified strain, and used to perform PCR with the synthetic DNAs of SEQ ID NOS: 67 and 68 as the primers to confirm deletion of the NCgl0313 gene, and the strain was designated as FKFC11 strain.

### <2-3> Construction of FKFC14 Strain

(2256ΔvanABKΔNCgl0324ΔNCgl0313ΔNCgl2709 Strain)
<2-3-1> Construction of Plasmid pBS4SΔ2256adhA for Deletion of NCgl2709 Gene

PCR was performed by using the genomic DNA of the C. glutamicum 2256 strain as the template, and the synthetic DNAs of SEQ ID NOS: 69 and 70 as the primers to obtain a PCR product containing an N-terminus side coding region of the NCgl2709 gene. Separately, PCR was performed by using the genomic DNA of the C. glutamicum 2256 strain as the template, and the synthetic DNAs of SEQ ID NOS: 71 and 72 as the primers to obtain a PCR product containing a C-terminus side coding region of the NCgl2709 gene. The sequences of SEQ ID NOS: 70 and 71 are partially complementary to each other. Then, approximately equimolar amounts of the PCR product containing the N-terminus side coding region of the NCgl2709 gene and the PCR product containing the C-terminus side coding region of the NCgl2709 gene were mixed, and inserted into the pBS4S vector treated with BamHI and PstI by using In Fusion HD Cloning Kit (Clontech). With this DNA, competent cells of Escherichia coli JM109 (Takara Bio) were transformed, and the cells were applied to the LB medium containing 100 μM IPTG, 40 μg/mL of X-Gal, and 40 μg/mL of kanamycin, and cultured overnight. Then, white colonies that appeared were picked up, and separated into single colonies to obtain transformants. Plasmids were extracted from the obtained transformants, and one in which the target PCR product was inserted was designated as pBS4SΔ2256adhA.

### <2-3-2> Construction of FKFC14 Strain

(2256ΔvanABKΔNCgl0324ΔNCgl0313ΔNCgl2709 Strain)

Since pBS4SΔ2256adhA obtained above does not contain the region enabling autonomous replication of the plasmid in cells of coryneform bacteria, if coryneform bacteria are transformed with this plasmid, a strain in which this plasmid is incorporated into the genome by homologous recombination appears as a transformant, although it occurs at an extremely low frequency. Therefore, pBS4SΔ2256adhA was introduced into the C. glutamicum FKFC11 strain by the electric pulse method. The cells were applied to the CM-Dex agar medium containing 25 μg/mL of kanamycin, and cultured at 31.5° C. It was confirmed by PCR that the grown strain was a once-recombinant strain in which pBS4SΔ2256adhA was incorporated into the genome by homologous recombination. This once-recombinant strain had both the wild-type NCgl2709 gene, and the deficient-type NCgl2709 gene.

The once-recombinant strain was cultured overnight in the CM-Dex liquid medium, the culture broth was applied to the S10 agar medium, and culture was performed at 31.5° C. Among the colonies that appeared, a strain that showed kanamycin susceptibility was purified on the CM-Dex agar medium. Genomic DNA was prepared from the purified strain, and used to perform PCR with the synthetic DNAs of SEQ ID NOS: 73 and 74 as the primers to confirm deletion of the NCgl2709 gene, and the strain was designated as FKFC14 strain.

<3> Construction of strain deficient in protocatechuic acid dioxygenase genes (FKFC14ΔpcaGH strain)

Subsequently, by using the Corynebacterium glutamicum FKFC14 strain as a parent strain, there was constructed a strain FKFC14ΔpcaGH, which is deficient in NCgl2314 gene (pcaG) and NCgl2315 gene (pcaH) encoding the alpha subunit and beta subunit of protocatechuate 3,4-dioxygenase, by outsourcing. The FKFC14ΔpcaGH strain can also be constructed via the following procedure.

<3-1> Construction of Plasmid pBS4SΔ2256pcaGH for Deletion of NCgl2314 and NCgl2315 Genes

NCgl2314 and NCgl2315 genes are adjacent to each other, and therefore these genes can be deleted all together. PCR is performed by using the genomic DNA of the C. glutamicum 2256 strain as the template, and the synthetic DNAs of SEQ ID NOS: 75 and 76 as the primers to obtain a PCR product containing an upstream region of the NCgl2315 gene. Separately, PCR is performed by using the genomic DNA of the C. glutamicum 2256 strain as the template, and the synthetic DNAs of SEQ ID NOS: 77 and 78 as the primers to obtain a PCR product containing a downstream region of the NCgl2314 gene. The sequences of SEQ ID NOS: 76 and 77 are partially complementary to each other. Then, approximately equimolar amounts of the PCR product containing the upstream region of the NCgl2315 gene and the PCR product containing the downstream region of the NCgl2314 gene are mixed, and inserted into the pBS4S vector (WO2007/046389) treated with BamHI and PstI by using In Fusion HD Cloning Kit (Clontech). With this DNA, competent cells of Escherichia coli JM109 (Takara Bio) are transformed, and the cells are applied to the LB medium containing 100 μM IPTG, 40 μg/mL of X-Gal, and 40 μg/mL of kanamycin, and cultured overnight. Then, white colonies that appeared are picked up, and separated into single colonies to obtain transformants. Plasmids are extracted from the obtained transformants, and one in which the target PCR product is inserted is designated as pBS4SΔ2256pcaGH.

### <3-2> Construction of FKFC14ΔpcaGH Strain

Since pBS4SΔ2256pcaGH obtained above does not contain the region enabling autonomous replication of the plasmid in cells of coryneform bacteria, if coryneform bacteria are transformed with this plasmid, a strain in which this plasmid is incorporated into the genome by homologous recombination appears as a transformant, although it occurs at an extremely low frequency. Therefore, pBS4SΔ2256pcaGH is introduced into the C. glutamicum FKFC14 strain by the electric pulse method. The cells are applied to the CM-Dex agar medium containing 25 μg/mL of kanamycin, and cultured at 31.5° C. It is confirmed by PCR that the grown strain is a once-recombinant strain in which pBS4SΔ2256pcaGH is incorporated into the genome by homologous recombination. This once-recombinant strain has both the wild-type NCgl2314 and NCgl2315 genes, and the deficient-type NCgl2314 and NCgl2315 genes.

The once-recombinant strain is cultured overnight in the CM-Dex liquid medium, the culture medium is applied to the S10 agar medium, and culture is performed at 31.5° C. Among the colonies that appear, a strain that shows kanamycin susceptibility is purified on the CM-Dex agar medium. Genomic DNA is prepared from the purified strain, and used to perform PCR with the synthetic DNAs of SEQ ID NOS: 79 and 80 as the primers to confirm deletion of the NCgl2314 and NCgl2315 genes, and the strain is designated as FKFC14ΔpcaGH strain.

### <4> Construction of Ap1-0112 Strain (FKFC14ΔpcaGH P8::NCgl2048 Strain)

Subsequently, by using the Corynebacterium glutamicum FKFC14ΔpcaGH strain as a parent strain, there was constructed a strain Ap1-0112, in which the promoter region of NCgl2048 gene has been replaced with the P8 promoter, by outsourcing. The nucleotide sequence of a genomic region containing the P8 promoter in this strain is shown as SEQ ID NO: 83, wherein position 901-1046 corresponds to the P8 promoter. The Ap1-0112 strain can also be constructed via the following procedure.

<4-1> Construction of Plasmid pBS4SP8::NCgl2048 for substitution of NCgl2048 Gene Promoter

PCR is performed by using the genomic DNA of the C. glutamicum 2256 strain as the template, and the synthetic DNAs of SEQ ID NOS: 85 and 86 as the primers to obtain a PCR product containing an upstream region of the NCgl2048 gene. Separately, PCR is performed by using the genomic DNA of the C. glutamicum 2256 strain as the template, and the synthetic DNAs of SEQ ID NOS: 87 and 88 as the primers to obtain a PCR product containing an N-terminus side coding region of the NCgl2048 gene. In addition, a DNA fragment of SEQ ID NO: 89 containing P8 promoter region is obtained by artificial gene synthesis. And then, PCR is performed by using the DNA fragment of SEQ ID NO: 89 as the template, and the synthetic DNAs of SEQ ID NOS: 90 and 91 as the primers to obtain a PCR product containing the P8 promoter. The sequences of SEQ ID NOS: 86 and 90 are partially complementary to each other, and the sequences of SEQ ID NOS: 87 and 91 are partially complementary to each other. Then, approximately equimolar amounts of the PCR product containing the upstream region of the NCgl2048 gene, the PCR product containing the N-terminus side coding region of the NCgl2048 gene, and the PCR product containing the P8 promoter are mixed, and inserted into the pBS4S vector (WO2007/046389) treated with BamHI and PstI by using In Fusion HD Cloning Kit (Clontech). With this DNA, competent cells of Escherichia coli JM109 (Takara Bio) are transformed, and the cells are applied to the LB medium containing 100 μM IPTG, 40 μg/mL of X-Gal, and 40 μg/mL of kanamycin, and cultured overnight. Then, white colonies that appeared are picked up, and separated into single colonies to obtain transformants. Plasmids are extracted from the obtained transformants, and one in which the target PCR product is inserted is designated as pBS4SP8::NCgl2048.

### <4-2> Construction of Ap1-0112 Strain

Since pBS4SP8::NCgl2048 obtained above does not contain the region enabling autonomous replication of the plasmid in cells of coryneform bacteria, if coryneform bacteria are transformed with this plasmid, a strain in which this plasmid is incorporated into the genome by homologous recombination appears as a transformant, although it occurs at an extremely low frequency. Therefore, pBS4SP8::NCgl2048 is introduced into the C. glutamicum FKFC14ΔpcaGH strain by the electric pulse method. The cells are applied to the CM-Dex agar medium containing 25 μg/mL of kanamycin, and cultured at 31.5° C. It is confirmed by PCR that the grown strain is a once-recombinant strain in which pBS4SP8::NCgl2048 is incorporated into the genome by homologous recombination.

The once-recombinant strain is cultured overnight in the CM-Dex liquid medium, the culture medium is applied to the S10 agar medium, and culture is performed at 31.5° C. Among the colonies that appear, a strain that shows kanamycin susceptibility is purified on the CM-Dex agar medium. Genomic DNA is prepared from the purified strain, and used to perform nucleotide sequence analysis to confirm that P8 promoter is located upstream of the NCgl2048 gene, and the strain is designated as Ap1-0112 strain.

<5> Construction of plasmid pVK9::PcspB-hsomt for Expression of OMT Gene of Homo sapiens
<5-1> Construction of Plasmid pEPlac-COMT2

Two kinds of OMT isoforms, i.e. shorter OMT isoform (S-COMT) and longer OMT isoform (MB-COMT), are known for the OMT gene of Homo sapiens. The amino acid sequence of S-COMT is shown as SEQ ID NO: 16, and the nucleotide sequence of wild-type cDNA encoding S-COMT is shown as SEQ ID NO: 94. The wild-type cDNA of S-COMT was codon-optimized for the expression in Escherichia coli (E. coli) and chemically synthesized using the service provided by ATG Service Gen (Russian Federation, Saint-Petersburg). To facilitate further cloning, the DNA fragment of gene was synthesized with sites for the restriction enzymes NdeI and SacI at 3′ and 5′ ends respectively. The codon-optimized S-COMT cDNA can also be referred to as COMT2 gene. The nucleotide sequence of the synthesized DNA fragment containing the COMT2 gene is shown as SEQ ID NO: 95. The synthesized DNA fragment including the COMT2 gene was obtained in pUC57 vector (GenScript).

The expression of the COMT2 gene was confirmed in the T7 system. The COMT2 gene inserted in pUC57 vector was re-cloned into NdeI and SacI restriction sites of pET22(+) vector (Novagen). The obtained plasmid was introduced into E. coli BL21(DE3) cells (Novagen). Cells containing the plasmid were grown in LB medium (Tryptone, 10 g/l; yeast extract, 5 g/l; NaCl, 10 g/l) containing ampicillin, 200 mg/l, and induced by IPTG, 1 mM within 2 h in the exponential phase of growth. Cells were disrupted by sonication. The crude protein extracts were analyzed using electrophoresis in 12% SDS-PAGE. The bands corresponding to S-OMT (about 24 kDa) was identified and cut out from the gel. The objective protein was isolated from gel and treated with trypsin. The obtained tryptic hydrolysates were analyzed using mass-spectroscopy to confirm the expression of the COMT2 gene.

The COMT2 gene inserted in pUC57 vector was re-cloned into the NdeI and Sad restriction sites of the pELAC vector (SEQ ID NO: 96, Smirnov S. V. et al., Appl. Microbiol. Biotechnol,. 2010, 88(3):719-726). The pELAC vector was constructed by replacing BglII-XbaI-fragment of pET22b(+) (Novagen) with synthetic BglII-XbaI-fragment containing P_{lacUV5} promoter. To insert the COMT2 gene into the pELAC vector, ligation reaction using T4 DNA ligase (Fermentas, Lithuania) was performed as recommended by the supplier. The ligation mixture was treated with ethanol, and the obtained precipitate was dissolved in water and introduced into E. coli TG1 cells using electroporation (Micro Pulser, BioRad) under the conditions recommended by the supplier. The cells were applied onto LA plates supplemented with ampicillin (200 mg/L) (Sambrook J. and Russell D. W., Molecular Cloning: A Laboratory Manual (3^{rd} ed.), Cold Spring Harbor Laboratory Press, 2001) and cultured overnight at 37° C. The obtained colonies were tested using PCR analysis to select the required clones. Primers P1 and P2 (SEQ ID NOS: 97 and 98) were used to select colonies containing the COMT2 gene. A DNA-fragment (713 bp) was obtained when vector-specific primer P1 and the reverse primer P2 for the ending of the COMT2 gene were used. Thus, the vector pEPlac-COMT2 was constructed. The sequence of the cloned COMT2 gene was determined using primers P1 and P3 (SEQ ID NOS: 97 and 99).

<5-2> Construction of Plasmid pVK9::PcspB-hsomt

PCR was performed by using the genomic DNA of the C. glutamicum 2256 strain as the template, and the synthetic DNAs of SEQ ID NOS: 100 and 101 as the primers to obtain a PCR product containing a PCR product containing a promoter region and SD sequence of cspB gene. Separately, PCR was also performed by using the plasmid pEPlac-COMT2 as the template, and the synthetic DNAs of SEQ ID NOS: 102 and 103 as the primers to obtain a PCR product containing the COMT2 gene. Then, these PCR products were inserted into the pVK9 vector (WO2007/046389) treated with BamHI and PstI by using In Fusion HD Cloning Kit (Clontech). The pVK9 vector is a shuttle-vector for coryneform bacteria and Escherichia coli. With this DNA, competent cells of Escherichia coli JM109 (Takara Bio) were transformed, and the cells were applied to the LB medium containing 100 μM IPTG, 40 μg/mL of X-Gal, and 25 μg/mL of kanamycin, and cultured overnight. Then, white colonies that appeared were picked up, and separated into single colonies to obtain transformants. Plasmids were extracted from the obtained transformants, and one into which the target PCR product was inserted was designated as pVK9::PcspB-hsomt.

### <6> Construction of Vanillic Acid-Producing Strains

The C. glutamicum FKFC14ΔpcaGH/pVK9::PcspB-hsomt and Ap1-0112/pVK9::PcspB-hsomt strains, which harbor the plasmid pVK9::PcspB-hsomt, were constructed by outsourcing. These strains can also be constructed via the following procedure.

The plasmid pVK9::PcspB-hsomt is introduced into the C. glutamicum FKFC14ΔpcaGH and Ap1-0112 strains by the electric pulse method. The cells are applied to the CM-Dex agar medium containing 25 μg/mL of kanamycin, and cultured at 31.5° C. The grown strains are purified on the same agar medium, and designated as FKFC14ΔpcaGH/pVK9::PcspB-hsomt and Ap1-0112/pVK9::PcspB-hsomt, respectively.

These strains were each inoculated into 4 mL of the CM-Dex w/o mameno medium (5 g/L of glucose, 10 g/L of Polypeptone, 10 g/L of Yeast Extract, 1 g/L of KH₂PO₄, 0.4 g/L of MgSO₄-7H₂O, 0.01 g/L of FeSO₄-7H₂O, 0.01 g/L of MnSO₄-7H₂O, 3 g/L of urea, 10 μg/L of biotin, adjusted to pH 7.5 with KOH) containing 25 μg/mL of kanamycin present in a test tube, and cultured at 31.5° C. with shaking for about 16 hr. A 0.9 mL aliquot of the obtained culture broth was mixed with 0.6 mL of 50% glycerol aqueous solution to obtain a glycerol stock, and stored at −80° C.

<7> Vanillic Acid Production by C. glutamicum
FKFC14ΔpcaGH/pVK9::PcspB-hsomt and Ap1-0112/pVK9::PcspB-hsomt strains

A 5 μL aliquot of each of the glycerol stocks of the FKFC14ΔpcaGH/pVK9::PcspB-hsomt and Ap1-0112/pVK9::PcspB-hsomt strains was inoculated into 4 mL of the CM-Dex w/o mameno medium containing 25 μg/mL of kanamycin present in a test tube, and cultured at 31.5° C. with shaking for 20 hr as preculture. A 0.5 mL aliquot of the obtained preculture broth was inoculated into 50 mL of the CM-Dex w/o mameno medium containing 25 μg/mL of kanamycin present in a conical flask with baffles, and cultured at 31.5° C. with shaking for 20 hr. The obtained culture broth was centrifuged at 8000 rpm for 5 minutes, the supernatant was removed, and the cells were suspended in sterilized physiological saline. The optical density (OD) of the cell suspension was measured, and the cell suspension was diluted with physiological saline to obtain an OD at 600 nm of 50. A 5 mL aliquot of the diluted cell suspension was inoculated into 20 mL of a vanillic acid production medium (75 g/L of glucose, 0.6 g/L of MgSO₄-7H₂O, 6.3 g/L of (NH₄)₂SO₄, 2.5 g/L of KH₂PO₄, 12.5 mg/L of FeSO₄-7H₂O, 12.5 mg/L of MnSO₄-4-5H₂O, 2.5 g/L of Yeast Extract, 150 μg/L of Vitamin B1, 150 μg/L of Biotin, 6.9 g/L of Protocatechuic acid, adjusted to pH 7 with KOH, and then mixed with 37.5 g/L of CaCO₃ (sterilized with hot air at 180° C. for 3 hours)) containing 25 μg/mL of kanamycin present in a conical flask with baffles, and cultured at 31.5° C. with shaking for 24 hr.

At the start and completion of the culture, the concentration of glucose in the medium was analyzed with Biotech Analyzer AS-310 (Sakura SI). The concentrations of protocatechuic acid and vanillic acid in the medium were also analyzed by using Ultra Performance Liquid Chromatography NEXERA X2 System (SHIMADZU) with the following conditions.

Conditions of UPLC analysis:

Column: KINETEX 2.6 μm XB-C18, 150×30 mm (Phenomenex)

Oven temperature: 40° C.

Mobile phase (A): 0.1% Trifluoroacetic acid

Mobile phase (B): 0.1% Trifluoroacetic acid/80% acetonitrile

Gradient program (time, A (%), B (%)): (0, 90, 10)−>(3, 80, 20)

Flow rate: 1.5 ml/min

The results are shown in Table 1. The vanillic acid concentration in the medium observed for the Ap1-0112/pVK9::PcspB-hsomt strain was about 1.2 times as high as that observed for the FKFC14ΔpcaGH/pVK9::PcspB-hsomt strain.

TABLE 1
Vanillic acid production by C. glutamicum vanillic acid-producing strains

At the start of culture

| | Concentration of | Concentration of |
|---|---|---|
| | glucose | protocatechuic acid |
| Strain | (g/L) | (g/L) |
| FKFC14ΔpcaGH/ | 57.8 ± 0.5 | 5.70 ± 0.2 |
| pVK9::PcspB-hsomt | | |
| Ap1_0112/ | 61.3 ± 0.3 | 5.67 ± 0.1 |
| pVK9::PcspB-hsomt | | |

| | At the completion of culture | | |
|---|---|---|---|
| | | Concentration | Concentration |
| | Concentration | of residual | of generated |
| | of residual | protocatechuic | vanillic |
| | glucose | acid | acid |
| Strain | (g/L) | (g/L) | (mg/L) |
| FKFC14ΔpcaGH/ | 13.0 ± 0.2 | 5.64 ± 0.1 | 73.0 ± 1.8 |
| pVK9::PcspB-hsomt | | | |
| Ap1_0112/ | 19.3 ± 1.2 | 5.66 ± 0.2 | 88.1 ± 5.0 |
| pVK9::PcspB-hsomt | | | |

### <8> Analysis of Expression Amount of NCgl2048 Gene by Quantitative PCR

Subsequently, the expression amount of NCgl2048 gene in the FKFC14ΔpcaGH/pVK9::PcspB-hsomt and Ap1-0112/pVK9::PcspB-hsomt strains were analyzed by quantitative PCR.

### <8-1> Preparation of RNA

A 250 μL aliquot of the culture broth containing cells, which culture broth was obtained 5 hr after the start of the culture in Example <7> for each of the FKFC14ΔpcaGH/pVK9::PcspB-hsomt and Ap1-0112/pVK9::PcspB-hsomt strains, was mixed with 500 μL of RNA Protect Bacteria Reagent (QIAGEN), and stored at −80° C. The frozen mixture was thawed at a room temperature, added with 200 μL of TE buffer (10 mM of Tris, 1 mM of EDTA, pH 8.0) containing lysozyme and with 10 μL of protease K (20 mg/mL), mixed, and then incubated at a room temperature for 40 min. The following procedure was performed using RNeasy Mini Kit (QIAGEN). The treated product was added with 700 μL of RLT buffer containing 1% of 2-mercaptoethanol, mixed, and centrifuged to obtain a supernatant. The supernatant was added with 500 μL of ethanol, mixed, and applied to a column included in the kit, and the column was centrifuged. The column was washed with 350 μL of RW1 buffer, and then 80 μL of DNasel solution was applied to the column to perform DNase treatment at a room temperature for 15 min. Furthermore, the column was washed with 350 μL of RW1 buffer and twice with 500 μL of RPE buffer, and eluted with RNase-free sterilized water to obtain RNA. The obtained RNA was quantified using NanoDrop (Thermo Fisher Scientific) and analyzed by electrophoresis using BioAnalyer (Agilent Technologies) with RNA 6000 Nano Kit (Agilent Technologies) to confirm that the obtained RNA had a sufficient purity.

<8-2> Synthesis of cDNA by Reverse Transcription

PrimeScript RT Reagent Kit with gDNA Eraser (TAKARA BIO) was used for reverse transcription. A 1 μg aliquot of RNA was added with 1 μL of gDNA Eraser and 2 μL of 5×DNA Eraser Buffer, diluted with sterilized water up to a total volume of 10 μL, and incubated at 42° C. for 2 min to degrade the chromosomal DNA. The resultant mixture was further added with 4 μL of 5×PrimeScript Buffer2, 1 μL of PrimeScript RT Enzyme MixI, 1 μL of RT Primer Mix, and 4 μL of sterilized water, incubated at 37° C. for 15 min and 85° C. for 5 sec to obtain cDNA.

### <8-3> Quantitative PCR

NCgl2048 gene was amplified as the target gene from cDNA with the following procedure: 2 μL of cDNA, 10 μL of Power SYBR Green PCR Master Mix (Life Technologies), primers of SEQ ID NOS: 104 and 105 (500 nM each as the final concentration), and sterilized water were mixed to obtain a total volume of 20 μL; PCR was performed with denaturation at 95° C. for 10 min followed by 40 cycles of 95° C. for 15 sec and 60° C. for 1 min using 7000 Real Time PCR system (Applied Bio Systems). In addition, 16S rRNA gene was amplified as a housekeeping gene from cDNA with the same procedure as that used for the target gene amplification, except that 2 μL of 32-fold diluted cDNA was used as the template and primers of SEQ ID NOS: 106 and 107 were used. After the amplification reaction, the PCR product was subjected to the melting curve analysis to confirm the uniformity of the PCR product.

Furthermore, the PCR product was analyzed by agarose gel electrophoresis to confirm that the PCR product had a length obtainable with the primers used.

### <8-4> Analysis of Expression Amount

The ΔΔCt method (METHODS, 25, 402(2001)) was used for analysis of the expression amount of NCgl2048 gene. A value obtained by subtracting the Ct value of the housekeeping gene from the Ct value of NCgl2048 gene was provided as ΔCt value. However, as the Ct value of the housekeeping gene, a value obtained by adding 5 to the actually measured ΔCt value of the housekeeping gene was used, because 32-fold diluted, that is, 2⁵-fold diluted, cDNA was used as the template for amplification of the housekeeping gene. A value obtained by subtracting the ΔCt value of the FKFC14ΔpcaGH/pVK9::PcspB-hsomt strain from the ΔCt value of the Ap1-0112/pVK9::PcspB-hsomt strain was provided as ΔΔCt value. The relative expression amount of NCgl2048 gene in the Ap1-0112/pVK9::PcspB-hsomt strain based on the FKFC14ΔpcaGH/pVK9::PcspB-hsomt strain was calculated as 2^{-ΔΔCt}.

The results are shown in Table2. The relative expression amount of NCgl2048 gene in the Ap1-0112/pVK9::PcspB-hsomt strain was approximately one twenty-fifth (1/25) of that in the FKFC14ΔpcaGH/pVK9::PcspB-hsomt strain.

TABLE 2
Relative expression amount of NCgl2048 gene

| Strain | 2^{-ΔΔCt} |
|---|---|
| FKFC14ΔpcaGH/pVK9::PcspB-hsomt | 1.00 |
| Ap1_0112/pVK9::PcspB-hsomt | 0.04 |

### INDUSTRIAL APPLICABILITY

According to the present invention, an ability of a microorganism for producing an objective substance such as vanillin and vanillic acid can be improved, and the objective substance can be efficiently produced.

### <Explanation of Sequence Listing>

### SEQ ID NOS:

1: Nucleotide sequence of aroG gene of Escherichia coli MG1655

2: Amino acid sequence of AroG protein of Escherichia coli MG1655

3: Nucleotide sequence of aroB gene of Escherichia coli MG1655

4: Amino acid sequence of AroB protein of Escherichia coli MG1655

5: Nucleotide sequence of aroD gene of Escherichia coli MG1655

6: Amino acid sequence of AroD protein of Escherichia coli MG1655

7: Nucleotide sequence of asbF gene of Bacillus thuringiensis BMB171

8: Amino acid sequence of AsbF protein of Bacillus thuringiensis BMB171

9: Nucleotide sequence of tyrR gene of Escherichia coli MG1655

10: Amino acid sequence of TyrR protein of Escherichia coli MG1655

11-14: Nucleotide sequences of transcript variants 1 to 4 of OMT gene of Homo sapiens

15: Amino acid sequence of OMT isoform (MB-COMT) of Homo sapiens

16: Amino acid sequence of OMT isoform (S-COMT) of Homo sapiens

17: Nucleotide sequence of ACAR gene of Nocardia brasiliensis

18: Amino acid sequence of ACAR protein of Nocardia brasiliensis

19: Nucleotide sequence of ACAR gene of Nocardia brasiliensis

20: Amino acid sequence of ACAR protein of Nocardia brasiliensis

21: Nucleotide sequence of entD gene of Escherichia coli MG1655

22: Amino acid sequence of EntD protein of Escherichia coli MG1655

23: Nucleotide sequence of PPT gene of Corynebacterium glutamicum ATCC 13032

24: Amino acid sequence of PPT protein of Corynebacterium glutamicum ATCC 13032

25: Nucleotide sequence of vanK gene of Corynebacterium glutamicum 2256 (ATCC 13869)

26: Amino acid sequence of VanK protein of Corynebacterium glutamicum 2256 (ATCC 13869)

27: Nucleotide sequence of pcaK gene of Corynebacterium glutamicum 2256 (ATCC 13869)

28: Amino acid sequence of PcaK protein of Corynebacterium glutamicum 2256 (ATCC 13869)

29: Nucleotide sequence of vanA gene of Corynebacterium glutamicum 2256 (ATCC 13869)

30: Amino acid sequence of VanA protein of Corynebacterium glutamicum 2256 (ATCC 13869)

31: Nucleotide sequence of vanB gene of Corynebacterium glutamicum 2256 (ATCC 13869)

32: Amino acid sequence of VanB protein of Corynebacterium glutamicum 2256 (ATCC 13869)

33: Nucleotide sequence of pcaG gene of Corynebacterium glutamicum ATCC 13032

34: Amino acid sequence of PcaG protein of Corynebacterium glutamicum ATCC 13032

35: Nucleotide sequence of pcaH gene of Corynebacterium glutamicum ATCC 13032

36: Amino acid sequence of PcaH protein of Corynebacterium glutamicum ATCC 13032

37: Nucleotide sequence of yqhD gene of Escherichia coli MG1655

38: Amino acid sequence of YqhD protein of Escherichia coli MG1655

39: Nucleotide sequence of NCgl0324 gene of Corynebacterium glutamicum 2256 (ATCC 13869)

40: Amino acid sequence of NCgl0324 protein of Corynebacterium glutamicum 2256 (ATCC 13869)

41: Nucleotide sequence of NCgl0313 gene of Corynebacterium glutamicum 2256 (ATCC 13869)

42: Amino acid sequence of NCgl0313 protein of Corynebacterium glutamicum 2256 (ATCC 13869)

43: Nucleotide sequence of NCgl2709 gene of Corynebacterium glutamicum 2256 (ATCC 13869)

44: Amino acid sequence of NCgl2709 protein of Corynebacterium glutamicum 2256 (ATCC 13869)

45: Nucleotide sequence of NCgl0219 gene of Corynebacterium glutamicum ATCC 13032

46: Amino acid sequence of NCgl0219 protein of Corynebacterium glutamicum ATCC 13032

47: Nucleotide sequence of NCgl2382 gene of Corynebacterium glutamicum ATCC 13032

48: Amino acid sequence of NCgl2382 protein of Corynebacterium glutamicum ATCC 13032

49: Nucleotide sequence of aroE gene of Escherichia coli MG1655

50: Amino acid sequence of AroE protein of Escherichia coli MG1655

51-80: Primers

81: Nucleotide sequence containing P2 promoter

82: Nucleotide sequence containing P4 promoter

83: Nucleotide sequence containing P8 promoter

84: Nucleotide sequence containing P3 promoter

85-88: Primers

89: Nucleotide sequence of DNA fragment containing P8 promoter region

90-91: Primers

92: Nucleotide sequence of NCgl2048 gene of Corynebacterium glutamicum 2256 (ATCC 13869)

93: Amino acid sequence of NCgl2048 protein of Corynebacterium glutamicum 2256 (ATCC 13869)

94: Nucleotide sequence of cDNA encoding S-COMT of Homo sapiens

95: Nucleotide sequence of synthesized DNA fragment containing COMT2 gene

96: pELAC vector

97-107: Primers

1. A method for producing an objective substance, the method comprising the following step:
producing the objective substance by using a microorganism having an ability to produce the objective substance, wherein the microorganism has been modified so that the activity of a protein encoded by NCgl2048 gene is reduced as compared with a non-modified microorganism, and wherein the objective substance is selected from the group consisting of:
(X) metabolites the biosynthesis of which requires S-adenosylmethionine,
(Y) L-methionine, and
(Z) combinations thereof.

2. The method according to claim 1, wherein said producing comprises:
cultivating the microorganism in a culture medium containing a carbon source to produce and accumulate the objective substance in the culture medium.

3. The method according to claim 1, wherein said producing comprises:
converting a precursor of the objective substance into the objective substance by using the microorganism.

4. The method according to claim 3, wherein said converting comprises:
cultivating the microorganism in a culture medium containing the precursor to produce and accumulate the objective substance in the culture medium.

5. The method according to claim 3, wherein said converting comprises:
allowing cells of the microorganism to act on the precursor in a reaction mixture to produce and accumulate the objective substance in the reaction mixture.

6. The method according to claim 5, wherein the cells are cells present in a culture broth of the microorganism, cells collected from the culture broth, cells present in a processed product of the culture broth, cells present in a processed product of the collected cells, or a combination of these.

7. The method according to claim 3, wherein the precursor is selected from the group consisting of protocatechuic acid, protocatechualdehyde, L-tryptophan, L-histidine, L-phenylalanine, L-tyrosine, L-arginine, L-ornithine, glycine, and combinations thereof

8. The method according to claim 1, the method further comprising collecting the objective substance.

9. The method according to claim 1, wherein the NCgl2048 gene encodes a protein selected from the group consisting of:
(a) a protein comprising the amino acid sequence of SEQ ID NO: 93,
(b) a protein comprising the amino acid sequence of SEQ ID NO: 93 but that includes substitution, deletion, insertion, and/or addition of 1 to 10 amino acid residues, and wherein said protein has a property that a reduction in the activity of the protein in a microorganism results in an increased production of an objective substance, and
(c) a protein comprising an amino acid sequence having an identity of 90% or higher to the amino acid sequence of SEQ ID NO: 93, and wherein said protein has a property that a reduction in the activity of the protein in a microorganism results in an increased production of an objective substance.

10. The method according to claim 1, wherein the activity of the protein encoded by the NCgl2048 gene is reduced by attenuating the expression of the NCgl2048 gene, or by disrupting the NCgl2048 gene.

11. The method according to claim 10, wherein the expression of the NCgl2048 gene is attenuated by modifying an expression control sequence of the NCgl2048 gene.

12. The method according to claim 1, wherein the microorganism is a bacterium belonging to the family Enterobacteriaceae, a coryneform bacterium, or yeast.

13. The method according to claim 12, wherein the microorganism is a bacterium belonging to the genus Corynebacterium.

14. The method according to claim 13, wherein the microorganism is Corynebacterium glutamicum.

15. The method according to claim 12, wherein the microorganism is a bacterium belonging to the genus Escherichia.

16. The method according to claim 15, wherein the microorganism is Escherichia coli.

17. The method according to claim 1, wherein the metabolites (X) are selected from the group consisting of vanillin, vanillic acid, melatonin, ergothioneine, mugineic acid, ferulic acid, polyamine, guaiacol, 4-vinylguaiacol, 4-ethylguaiacol, and creatine.

18. The method according to claim 1, wherein the microorganism has been further modified so that the activity of an enzyme that is involved in the biosynthesis of the objective substance is increased as compared with a non-modified microorganism.

19. The method according to claim 18, wherein the enzyme that is involved in the biosynthesis of the objective substance is selected from the group consisting of 3-deoxy-D-arabino-heptulosonic acid 7-phosphate synthase, 3-dehydroquinate synthase, 3-dehydroquinate dehydratase, 3-dehydroshikimate dehydratase, O-methyltransferase, aromatic aldehyde oxidoreductase, and combinations thereof.

20. The method according to claim 1, wherein the microorganism has been further modified so that the activity of phosphopantetheinyl transferase is increased as compared with a non-modified microorganism.

21. The method according to claim 1, wherein the microorganism has been further modified so that the activity of an enzyme that is involved in the by-production of a substance other than the objective substance is reduced as compared with a non-modified microorganism.

22. The method according to claim 21, wherein the enzyme that is involved in the by-production of a substance other than the objective substance is selected from the group consisting of vanillate demethylase, protocatechuate 3,4-dioxygenase, alcohol dehydrogenase, shikimate dehydrogenase, and combinations thereof.

23. A method for producing vanillin, the method comprising:
producing vanillic acid by the method according to claim 1; and
converting said vanillic acid to vanillin.

24. The method according to claim 23, wherein the microorganism is a bacterium belonging to the genus Corynebacterium.

25. The method according to claim 23, wherein the microorganism is Corynebacterium glutamicum.


SEQUENCE LISTING (107 sequences)

SEQ ID NO: 1 - DNA, 1053 bp, Escherichia coli
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaa

SEQ ID NO: 2 - protein, 350 aa, Escherichia coli
MNYQNDDLRIKEIKELLPPVALLEKFPATENAANTVAHARKAIHKILKGNDDRLLVVIGPCSIHDPVAAKEYATRLLALREELKDELEIVMRVYFEKPRTTVGWKGLINDPHMDNSFQINDGLRIARKLLLDINDSGLPAAGEFLDMITPQYLADLMSWGAIGARTTESQVHRELASGLSCPVGFKNGTDGTIKVAIDAINAAGAPHCFLSVTKWGHSAIVNTSGNGDCHIILRGGKEPNYSAKHVAEVKEGLNKAGLPAQVMIDFSHANSSKQFKKQMDVCADVCQQIAGGEKAIIGVMVESHLVEGNQSLESGEPLAYGKSITDACIGWEDTDALLRQLANAVKARRG

SEQ ID NO: 3 - DNA, 1089 bp, Escherichia coli
atggagaggattgtcgttactctcggggaacgtagttacccaattaccatcgcatctggtttgtttaatgaaccagcttcattcttaccgctgaaatcgggcgagcaggtcatgttggtcaccaacgaaaccctggctcctctgtatctcgataaggtccgcggcgtacttgaacaggcgggtgttaacgtcgatagcgttatcctccctgacggcgagcagtataaaagcctggctgtactcgataccgtctttacggcgttgttacaaaaaccgcatggtcgcgatactacgctggtggcgcttggcggcggcgtagtgggcgatctgaccggcttcgcggcggcgagttatcagcgcggtgtccgtttcattcaagtcccgacgacgttactgtcgcaggtcgattcctccgttggcggcaaaactgcggtcaaccatcccctcggtaaaaacatgattggcgcgttctaccaacctgcttcagtggtggtggatctcgactgtctgaaaacgcttcccccgcgtgagttagcgtcggggctggcagaagtcatcaaatacggcattattcttgacggtgcgttttttaactggctggaagagaatctggatgcgttgttgcgtctggacggtccggcaatggcgtactgtattcgccgttgttgtgaactgaaggcagaagttgtcgccgccgacgagcgcgaaaccgggttacgtgctttactgaatctgggacacacctttggtcatgccattgaagctgaaatggggtatggcaattggttacatggtgaagcggtcgctgcgggtatggtgatggcggcgcggacgtcggaacgtctcgggcagtttagttctgccgaaacgcagcgtattataaccctgctcaagcgggctgggttaccggtcaatgggccgcgcgaaatgtccgcgcaggcgtatttaccgcatatgctgcgtgacaagaaagtccttgcgggagagatgcgcttaattcttccgttggcaattggtaagagtgaagttcgcagcggcgtttcgcacgagcttgttcttaacgccattgccgattgtcaatcagcgtaa

SEQ ID NO: 4 - protein, 362 aa, Escherichia coli
MERIVVTLGERSYPITIASGLFNEPASFLPLKSGEQVMLVTNETLAPLYLDKVRGVLEQAGVNVDSVILPDGEQYKSLAVLDTVFTALLQKPHGRDTTLVALGGGVVGDLTGFAAASYQRGVRFIQVPTTLLSQVDSSVGGKTAVNHPLGKNMIGAFYQPASVVVDLDCLKTLPPRELASGLAEVIKYGIILDGAFFNWLEENLDALLRLDGPAMAYCIRRCCELKAEVVAADERETGLRALLNLGHTFGHAIEAEMGYGNWLHGEAVAAGMVMAARTSERLGQFSSAETQRIITLLKRAGLPVNGPREMSAQAYLPHMLRDKKVLAGEMRLILPLAIGKSEVRSGVSHELVLNAIADCQSA

SEQ ID NO: 5 - DNA, 759 bp, Escherichia coli
atgaaaaccgtaactgtaaaagatctcgtcattggtacgggcgcacctaaaatcatcgtctcgctgatggcgaaagatatcgccagcgtgaaatccgaagctctcgcctatcgtgaagcggactttgatattctggaatggcgtgtggaccactatgccgacctctccaatgtggagtctgtcatggcggcagcaaaaattctccgtgagaccatgccagaaaaaccgctgctgtttaccttccgcagtgccaaagaaggcggcgagcaggcgatttccaccgaggcttatattgcactcaatcgtgcagccatcgacagcggcctggttgatatgatcgatctggagttatttaccggtgatgatcaggttaaagaaaccgtcgcctacgcccacgcgcatgatgtgaaagtagtcatgtccaaccatgacttccataaaacgccggaagccgaagaaatcattgcccgtctgcgcaaaatgcaatccttcgacgccgatattcctaagattgcgctgatgccgcaaagtaccagcgatgtgctgacgttgcttgccgcgaccctggagatgcaggagcagtatgccgatcgtccaattatcacgatgtcgatggcaaaaactggcgtaatttctcgtctggctggtgaagtatttggctcggcggcaacttttggtgcggtaaaaaaagcgtctgcgccagggcaaatctcggtaaatgatttgcgcacggtattaactattttacaccaggcataa

SEQ ID NO: 6 - protein, 252 aa, Escherichia coli
MKTVTVKDLVIGTGAPKIIVSLMAKDIASVKSEALAYREADFDILEWRVDHYADLSNVESVMAAAKILRETMPEKPLLFTFRSAKEGGEQAISTEAYIALNRAAIDSGLVDMIDLELFTGDDQVKETVAYAHAHDVKVVMSNHDFHKTPEAEEIIARLRKMQSFDADIPKIALMPQSTSDVLTLLAATLEMQEQYADRPIITMSMAKTGVISRLAGEVFGSAATFGAVKKASAPGQISVNDLRTVLTILHQA

SEQ ID NO: 7 - DNA, 843 bp, Bacillus thuringiensis
atgaaatattcgctatgtaccatttcatttcgtcatcaattaatttcatttactgatattgttcaatttgcatatgaaaacggttttgaaggaattgaattatgggggacgcatgcacaaaatttgtacatgcaagaacgtgaaacgacagaacgagaattgaattttctaaaggataaaaacttagaaattacgatgataagtgattacttagatatatcattatcagcagattttgaaaaaacgatagagaaaagtgaacaacttgtagtactagctaattggtttaatacgaataaaattcgcacgtttgctgggcaaaaagggagcaaggacttctcggaacaagagagaaaagagtatgtgaagcgaatacgtaagatttgtgatgtgtttgctcagaacaatatgtatgtgctgttagaaacacatcccaatacactaacggacacattgccttctactatagagttattagaagaagtaaaccatccgaatttaaaaataaatcttgattttcttcatatatgggagtctggcgcagatccaatagacagtttccatcgattaaagccgtggacactacattaccattttaagaatatatcttcagcggattatttgcatgtgtttgaacctaataatgtatatgctgcagcaggaagtcgtataggtatggttccgttatttgaaggtattgtaaattatgatgagattattcaggaagtgagaaatacggatctttttgcttccttagaatggtttggacataattcaaaagagatattaaaagaagaaatgaaagtattaataaatagaaaattagaagtagtaacttcgtaa

SEQ ID NO: 8 - protein, 280 aa, Bacillus thuringiensis
MKYSLCTISFRHQLISFTDIVQFAYENGFEGIELWGTHAQNLYMQERETTERELNFLKDKNLEITMISDYLDISLSADFEKTIEKSEQLVVLANWFNTNKIRTFAGQKGSKDFSEQERKEYVKRIRKICDVFAQNNMYVLLETHPNTLTDTLPSTIELLEEVNHPNLKINLDFLHIWESGADPIDSFHRLKPWTLHYHFKNISSADYLHVFEPNNVYAAAGSRIGMVPLFEGIVNYDEIIQEVRNTDLFASLEWFGHNSKEILKEEMKVLINRKLEVVTS

SEQ ID NO: 9 - DNA, 1542 bp, Escherichia coli
atgcgtctggaagtcttttgtgaagaccgactcggtctgacccgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtggatatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtgaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 10 - protein, 513 aa, Escherichia coli
MRLEVFCEDRLGLTRELLDLLVLRGIDLRGIEIDPIGRIYLNFAELEFESFSSLMAEIRRIAGVTDVRTVPWMPSEREHLALSALLEALPEPVLSVDMKSKVDMANPASCQLFGQKLDRLRNHTAAQLINGFNFLRWLESEPQDSHNEHVVINGQNFLMEITPVYLQDENDQHVLTGAVVMLRSTIRMGRQLQNVAAQDVSAFSQIVAVSPKMKHVVEQAQKLAMLSAPLLITGDTGTGKDLFAYACHQASPRAGKPYLALNCASIPEDAVESELFGHAPEGKKGFFEQANGGSVLLDEIGEMSPRMQAKLLRFLNDGTFRRVGEDHEVHVDVRVICATQKNLVELVQKGMFREDLYYRLNVLTLNLPPLRDCPQDIMPLTELFVARFADEQGVPRPKLAADLNTVLTRYAWPGNVRQLKNAIYRALTQLDGYELRPQDILLPDYDAATVAVGEDAMEGSLDEITSRFERSVLTQLYRNYPSTRKLAKRLGVSHTAIANKLREYGLSQKKNEE

SEQ ID NO: 11 - DNA, 2304 bp, Homo sapiens
cggcctgcgtccgccaccggaagcgccctcctaatccccgcagcgccaccgccattgccgccatcgtcgtggggcttctggggcagctagggctgcccgccgcgctgcctgcgccggaccggggcgggtccagtcccgggcgggccgtcgcgggagagaaataacatctgctttgctgccgagctcagaggagaccccagacccctcccgcagccagagggctggagcctgctcagaggtgctttgaagatgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctgactgcccccccggcccccctctcgggctctctcacccagcctggtactgaaggtgccagacgtgctcctgctgaccttctgcggctccgggctgtgtcctaaatgcaaagcacacctcggccgaggcctgcgccctgacatgctaacctctctgaactgcaacactggattgttcttttttaagactcaatcatgacttctttactaacactggctagctatattatcttatatactaatatcatgttttaaaaatataaaatagaaattaagaatctaaatatttagatataactcgacttagtacatccttctcaactgccattcccctgctgcccttgacttgggcaccaaacattcaaagctccccttgacggacgctaacgctaagggcggggcccctagctggctgggttctgggtggcacgcctggcccactggcctcccagccacagtggtgcagaggtcagccctcctgcagctaggccaggggcacctgttagccccatggggacgactgccggcctgggaaacgaagaggagtcagccagcattcacacctttctgaccaagcaggcgctggggacaggtggaccccgcagcagcaccagcccctctgggccccatgtggcacagagtggaagcatctccttccctactccccactgggccttgcttacagaagaggcaatggctcagaccagctcccgcatccctgtagttgcctccctggcccatgagtgaggatgcagtgctggtttctgcccacctacacctagagctgtccccatctcctccaaggggtcagactgctagccacctcagaggctccaagggcccagttcccaggcccaggacaggaatcaaccctgtgctagctgagttcacctgcaccgagaccagcccctagccaagattctactcctgggctcaaggcctggctagcccccagccagcccactcctatggatagacagaccagtgagcccaagtggacaagtttggggccacccagggaccagaaacagagcctctgcaggacacagcagatgggcacctgggaccacctccacccagggccctgccccagacgcgcagaggcccgacacaagggagaagccagccacttgtgccagacctgagtggcagaaagcaaaaagttcctttgctgctttaatttttaaattttcttacaaaaatttaggtgtttaccaatagtcttattttggcttatttttaa

SEQ ID NO: 12 - DNA, 2262 bp, Homo sapiens
ctcccacgggaggagcaagaacacagaacagagggggcaagacagctccaccaggagtcaggagtgaatcccctctgggaacgaggcactaggaagaagaacttccagcccaggagaaataacatctgctttgctgccgagctcagaggagaccccagacccctcccgcagccagagggctggagcctgctcagaggtgctttgaagatgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctgactgcccccccggcccccctctcgggctctctcacccagcctggtactgaaggtgccagacgtgctcctgctgaccttctgcggctccgggctgtgtcctaaatgcaaagcacacctcggccgaggcctgcgccctgacatgctaacctctctgaactgcaacactggattgttcttttttaagactcaatcatgacttctttactaacactggctagctatattatcttatatactaatatcatgttttaaaaatataaaatagaaattaagaatctaaatatttagatataactcgacttagtacatccttctcaactgccattcccctgctgcccttgacttgggcaccaaacattcaaagctccccttgacggacgctaacgctaagggcggggcccctagctggctgggttctgggtggcacgcctggcccactggcctcccagccacagtggtgcagaggtcagccctcctgcagctaggccaggggcacctgttagccccatggggacgactgccggcctgggaaacgaagaggagtcagccagcattcacacctttctgaccaagcaggcgctggggacaggtggaccccgcagcagcaccagcccctctgggccccatgtggcacagagtggaagcatctccttccctactccccactgggccttgcttacagaagaggcaatggctcagaccagctcccgcatccctgtagttgcctccctggcccatgagtgaggatgcagtgctggtttctgcccacctacacctagagctgtccccatctcctccaaggggtcagactgctagccacctcagaggctccaagggcccagttcccaggcccaggacaggaatcaaccctgtgctagctgagttcacctgcaccgagaccagcccctagccaagattctactcctgggctcaaggcctggctagcccccagccagcccactcctatggatagacagaccagtgagcccaagtggacaagtttggggccacccagggaccagaaacagagcctctgcaggacacagcagatgggcacctgggaccacctccacccagggccctgccccagacgcgcagaggcccgacacaagggagaagccagccacttgtgccagacctgagtggcagaaagcaaaaagttcctttgctgctttaatttttaaattttcttacaaaaatttaggtgtttaccaatagtcttattttggcttatttttaa

SEQ ID NO: 13 - DNA, 2279 bp, Homo sapiens
tggagataacacggatcgctgtgtacactgtgtgctccggttgttgcatccgagggttgatcggatggtggttcccatccagatccaagtcctggcccctgatcacagagaaacacagctggacattaaagtgaaataacatctgctttgctgccgagctcagaggagaccccagacccctcccgcagccagagggctggagcctgctcagaggtgctttgaagatgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctgactgcccccccggcccccctctcgggctctctcacccagcctggtactgaaggtgccagacgtgctcctgctgaccttctgcggctccgggctgtgtcctaaatgcaaagcacacctcggccgaggcctgcgccctgacatgctaacctctctgaactgcaacactggattgttcttttttaagactcaatcatgacttctttactaacactggctagctatattatcttatatactaatatcatgttttaaaaatataaaatagaaattaagaatctaaatatttagatataactcgacttagtacatccttctcaactgccattcccctgctgcccttgacttgggcaccaaacattcaaagctccccttgacggacgctaacgctaagggcggggcccctagctggctgggttctgggtggcacgcctggcccactggcctcccagccacagtggtgcagaggtcagccctcctgcagctaggccaggggcacctgttagccccatggggacgactgccggcctgggaaacgaagaggagtcagccagcattcacacctttctgaccaagcaggcgctggggacaggtggaccccgcagcagcaccagcccctctgggccccatgtggcacagagtggaagcatctccttccctactccccactgggccttgcttacagaagaggcaatggctcagaccagctcccgcatccctgtagttgcctccctggcccatgagtgaggatgcagtgctggtttctgcccacctacacctagagctgtccccatctcctccaaggggtcagactgctagccacctcagaggctccaagggcccagttcccaggcccaggacaggaatcaaccctgtgctagctgagttcacctgcaccgagaccagcccctagccaagattctactcctgggctcaaggcctggctagcccccagccagcccactcctatggatagacagaccagtgagcccaagtggacaagtttggggccacccagggaccagaaacagagcctctgcaggacacagcagatgggcacctgggaccacctccacccagggccctgccccagacgcgcagaggcccgacacaagggagaagccagccacttgtgccagacctgagtggcagaaagcaaaaagttcctttgctgctttaatttttaaattttcttacaaaaatttaggtgtttaccaatagtcttattttggcttatttttaa

SEQ ID NO: 14 - DNA, 2035 bp, Homo sapiens
gctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctgactgcccccccggcccccctctcgggctctctcacccagcctggtactgaaggtgccagacgtgctcctgctgaccttctgcggctccgggctgtgtcctaaatgcaaagcacacctcggccgaggcctgcgccctgacatgctaacctctctgaactgcaacactggattgttcttttttaagactcaatcatgacttctttactaacactggctagctatattatcttatatactaatatcatgttttaaaaatataaaatagaaattaagaatctaaatatttagatataactcgacttagtacatccttctcaactgccattcccctgctgcccttgacttgggcaccaaacattcaaagctccccttgacggacgctaacgctaagggcggggcccctagctggctgggttctgggtggcacgcctggcccactggcctcccagccacagtggtgcagaggtcagccctcctgcagctaggccaggggcacctgttagccccatggggacgactgccggcctgggaaacgaagaggagtcagccagcattcacacctttctgaccaagcaggcgctggggacaggtggaccccgcagcagcaccagcccctctgggccccatgtggcacagagtggaagcatctccttccctactccccactgggccttgcttacagaagaggcaatggctcagaccagctcccgcatccctgtagttgcctccctggcccatgagtgaggatgcagtgctggtttctgcccacctacacctagagctgtccccatctcctccaaggggtcagactgctagccacctcagaggctccaagggcccagttcccaggcccaggacaggaatcaaccctgtgctagctgagttcacctgcaccgagaccagcccctagccaagattctactcctgggctcaaggcctggctagcccccagccagcccactcctatggatagacagaccagtgagcccaagtggacaagtttggggccacccagggaccagaaacagagcctctgcaggacacagcagatgggcacctgggaccacctccacccagggccctgccccagacgcgcagaggcccgacacaagggagaagccagccacttgtgccagacctgagtggcagaaagcaaaaagttcctttgctgctttaatttttaaattttcttacaaaaatttaggtgtttaccaatagtcttattttggcttatttttaa

SEQ ID NO: 15 - protein, 271 aa, Homo sapiens
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

SEQ ID NO: 16 - protein, 221 aa, Homo sapiens
MGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

SEQ ID NO: 17 - DNA, 3453 bp, Nocardia brasiliensis
ttgttcgccgaggacgagcaggtgaaagccgcggtgccggaccaggaggtggtcgaggcgatccgggcgcccggcctgcgcctggcacagatcatggccaccgtgatggagcgctatgcggaccgccccgcggtgggacagcgggcgagcgagccggtcaccgagagcggtcgcaccaccttccggctgctcccggaattcgagaccctgacctaccgcgagctgtgggcgcgcgtccgcgcggtggccgccgcgtggcacggagatgccgaaaggcctttgcgggccggggatttcgttgctctgctgggtttcgccggcatcgattacggcaccctcgatctcgcgaacatccatctcggcctcgtcacggtgccgctgcaatccggcgccacggccccgcaactcgccgcgatcctggccgagaccacgccccgggtgctggccgcgacacccgaccatctcgatatcgccgtcgaattgctgaccgggggagcctcgccggaacggctggtggtattcgactaccgccccgcggacgacgatcaccgggcggcgctcgagtccgcgcgcagacggttgagcgacgcgggcagtgcggtggtggtcgagacgctcgacgcggtccgcgcccgcggcagcgaattgccggccgcgccgctgttcgttcccgccgcggacgaggacccgctggctctgctcatctacacctccggcagcaccggcacgcctaagggcgccatgtacaccgaaagactgaaccgcacgacgtggctgagcggggcgaaaggcgtcggcctcacgctcggctacatgccgatgagtcatattgccgggcgggcctcgttcgccggtgtgctggcccgcggcggcacggtctacttcaccgcccgcagcgatatgtcgacgctgttcgaagatctggccctggtgcggccgaccgagatgttcttcgtcccgcgcgtgtgcgacatgatcttccagcgctatcaggccgaactgtcgcggcgcgcgcccgccgcggccgcgagcccggaactcgagcaggaactgaagaccgaactgcgcttgtccgcggtcggggaccgcttactcggggcgatcgcgggcagcgcgccgctgtcggccgagatgcgggagttcatggagtcgctgctggatctggaactgcacgacggctacggctcgaccgaggcgggtatcggcgtactgcaagacaatatcgtccagcgtccgccggtcatcgattacaagctcgtcgacgtgccggaattgggctacttccggacggaccagccgcatccccgcggtgagttgctgttgaaaaccgaagggatgattccgggctacttccggcggcccgaggtgaccgcggagatcttcgacgaggacggtttctacaggaccggtgacatcgtcgccgaactcgaaccggatcggctgatctacctggaccgccgcaacaatgtgctgaaactggcccagggcgagttcgtcacggtcgcccatctggaagcggtgttcgcgaccagtccgctgatccggcagatctacatctacggcaacagcgagcgctcgttcctgctggcggtgatcgtgcccaccgcggacgcgctggccgacggtgtcaccgacgcgctgaacacggcgctgaccgaatccttgcgacagctcgcgaaagaagccgggctgcaatcctatgagctgccgcgcgagttcctggtcgaaaccgaaccgttcaccgtcgagaacggtctgctctccggtatcgcgaaactgttgcggcccaagctcaaggagcactacggcgagcgactcgagcagctgtaccgcgatatcgaggcgaaccgcaacgacgagctgatcgagctgcggcgcaccgcggccgagctgccggtgctcgaaaccgtcacgcgggctgcacgttcgatgctcggactggccgcgtcggagttgcggccggacgcgcatttcaccgatctcggcggtgattcactgtccgcgctgtcgttttcgaccctgctgcaggacatgctcgaggtcgaggtcccggtcggtgtcatcgtgagccccgccaactcgctcgccgatctggcgaaatacatcgaggccgaacggcattcgggggtgcggcggccgagcctgatctcggtgcacggtcccggcaccgagatccgtgccgccgatctcaccctggacaagttcatcgacgagcgcaccctcgctgccgcgaaagcggttccggccgcgccggcccaggcgcagaccgtcctgctcaccggggcgaacggctatctcggccgcttcctgtgcctggaatggctgcagcgactggaccagaccggcggcacgctggtctgcatcgtgcgcggtaccgacgcggccgccgcgcggaagcgcctggatgcggtgttcgacagcggtgatccggagctgctcgaccactaccggaagctggccgccgagcacctcgaggtgctcgcgggcgatatcggcgacccgaatctcggcctggacgaagcgacttggcagcggctcgccgcgaccgtcgacctgatcgtgcaccccgccgccctcgtcaaccatgtgctgccgtacagccagctgttcgggccgaatgtggtcggcaccgccgagatcatccggctggccatcaccgagcgccgtaagcccgtgacgtacctgtcgacggtcgcggtggccgcacaggtcgatcccgccggcttcgacgaggagcgcgatatccgggagatgagcgcggtgcgctccatcgacgccgggtacgcgaacggttacggcaacagcaagtgggccggcgaggtgctgctgcgcgaggcccatgatctgtgcgggctgccggtcgccgtgttccgctcggacatgatcctggcgcacagcaaatacgtcggtcagctcaacgtccccgatgtgttcacccggctcatcctgagcctggcgctcaccggcatcgcaccgtattcgttctacgggacggacagcgccgggcagcgcaggcgggcccactacgacggtctgcccgccgatttcgtcgccgaggcgatcaccaccctcggcgcgcgagccgagtcggggttccatacctacgacgtgtggaacccgtacgacgacggcatctcgctggacgaattcgtcgactggctcggcgatttcggcgtgccgatccagcggatcgacgactacgacgaatggttccggcgtttcgagaccgcgatccgcgcgctgcccgaaaagcagcgcgatgcttcgctgctaccgctgctggacgcacaccggcggccactgcgcgcggtgcgcggttcgctgttgcccgccaagaacttccaggcggcggtgcagtccgcgcggatcggccccgatcaggacatcccgcatctttccccgcagttgatcgacaagtacgtcaccgacctgcgccacctcggcctgctctga

SEQ ID NO: 18 - protein, 1150 aa, Nocardia brasiliensis
MFAEDEQVKAAVPDQEVVEAIRAPGLRLAQIMATVMERYADRPAVGQRASEPVTESGRTTFRLLPEFETLTYRELWARVRAVAAAWHGDAERPLRAGDFVALLGFAGIDYGTLDLANIHLGLVTVPLQSGATAPQLAAILAETTPRVLAATPDHLDIAVELLTGGASPERLVVFDYRPADDDHRAALESARRRLSDAGSAVVVETLDAVRARGSELPAAPLFVPAADEDPLALLIYTSGSTGTPKGAMYTERLNRTTWLSGAKGVGLTLGYMPMSHIAGRASFAGVLARGGTVYFTARSDMSTLFEDLALVRPTEMFFVPRVCDMIFQRYQAELSRRAPAAAASPELEQELKTELRLSAVGDRLLGAIAGSAPLSAEMREFMESLLDLELHDGYGSTEAGIGVLQDNIVQRPPVIDYKLVDVPELGYFRTDQPHPRGELLLKTEGMIPGYFRRPEVTAEIFDEDGFYRTGDIVAELEPDRLIYLDRRNNVLKLAQGEFVTVAHLEAVFATSPLIRQIYIYGNSERSFLLAVIVPTADALADGVTDALNTALTESLRQLAKEAGLQSYELPREFLVETEPFTVENGLLSGIAKLLRPKLKEHYGERLEQLYRDIEANRNDELIELRRTAAELPVLETVTRAARSMLGLAASELRPDAHFTDLGGDSLSALSFSTLLQDMLEVEVPVGVIVSPANSLADLAKYIEAERHSGVRRPSLISVHGPGTEIRAADLTLDKFIDERTLAAAKAVPAAPAQAQTVLLTGANGYLGRFLCLEWLQRLDQTGGTLVCIVRGTDAAAARKRLDAVFDSGDPELLDHYRKLAAEHLEVLAGDIGDPNLGLDEATWQRLAATVDLIVHPAALVNHVLPYSQLFGPNVVGTAEIIRLAITERRKPVTYLSTVAVAAQVDPAGFDEERDIREMSAVRSIDAGYANGYGNSKWAGEVLLREAHDLCGLPVAVFRSDMILAHSKYVGQLNVPDVFTRLILSLALTGIAPYSFYGTDSAGQRRRAHYDGLPADFVAEAITTLGARAESGFHTYDVWNPYDDGISLDEFVDWLGDFGVPIQRIDDYDEWFRRFETAIRALPEKQRDASLLPLLDAHRRPLRAVRGSLLPAKNFQAAVQSARIGPDQDIPHLSPQLIDKYVTDLRHLGLL

SEQ ID NO: 19 - DNA, 3501 bp, Nocardia brasiliensis
atggcgactgattcgcgaagcgatcggctacggcgtcgaattgcacagttgttcgccgaggacgagcaggtgaaagccgcggtgccggaccaggaggtggtcgaggcgatccgggcgcccggcctgcgcctggcacagatcatggccaccgtgatggagcgctatgcggaccgccccgcggtgggacagcgggcgagcgagccggtcaccgagagcggtcgcaccaccttccggctgctcccggaattcgagaccctgacctaccgcgagctgtgggcgcgcgtccgcgcggtggccgccgcgtggcacggagatgccgaaaggcctttgcgggccggggatttcgttgctctgctgggtttcgccggcatcgattacggcaccctcgatctcgcgaacatccatctcggcctcgtcacggtgccgctgcaatccggcgccacggccccgcaactcgccgcgatcctggccgagaccacgccccgggtgctggccgcgacacccgaccatctcgatatcgccgtcgaattgctgaccgggggagcctcgccggaacggctggtggtattcgactaccgccccgcggacgacgatcaccgggcggcgctcgagtccgcgcgcagacggttgagcgacgcgggcagtgcggtggtggtcgagacgctcgacgcggtccgcgcccgcggcagcgaattgccggccgcgccgctgttcgttcccgccgcggacgaggacccgctggctctgctcatctacacctccggcagcaccggcacgcctaagggcgccatgtacaccgaaagactgaaccgcacgacgtggctgagcggggcgaaaggcgtcggcctcacgctcggctacatgccgatgagtcatattgccgggcgggcctcgttcgccggtgtgctggcccgcggcggcacggtctacttcaccgcccgcagcgatatgtcgacgctgttcgaagatctggccctggtgcggccgaccgagatgttcttcgtcccgcgcgtgtgcgacatgatcttccagcgctatcaggccgaactgtcgcggcgcgcgcccgccgcggccgcgagcccggaactcgagcaggaactgaagaccgaactgcgcttgtccgcggtcggggaccgcttactcggggcgatcgcgggcagcgcgccgctgtcggccgagatgcgggagttcatggagtcgctgctggatctggaactgcacgacggctacggctcgaccgaggcgggtatcggcgtactgcaagacaatatcgtccagcgtccgccggtcatcgattacaagctcgtcgacgtgccggaattgggctacttccggacggaccagccgcatccccgcggtgagttgctgttgaaaaccgaagggatgattccgggctacttccggcggcccgaggtgaccgcggagatcttcgacgaggacggtttctacaggaccggtgacatcgtcgccgaactcgaaccggatcggctgatctacctggaccgccgcaacaatgtgctgaaactggcccagggcgagttcgtcacggtcgcccatctggaagcggtgttcgcgaccagtccgctgatccggcagatctacatctacggcaacagcgagcgctcgttcctgctggcggtgatcgtgcccaccgcggacgcgctggccgacggtgtcaccgacgcgctgaacacggcgctgaccgaatccttgcgacagctcgcgaaagaagccgggctgcaatcctatgagctgccgcgcgagttcctggtcgaaaccgaaccgttcaccgtcgagaacggtctgctctccggtatcgcgaaactgttgcggcccaagctcaaggagcactacggcgagcgactcgagcagctgtaccgcgatatcgaggcgaaccgcaacgacgagctgatcgagctgcggcgcaccgcggccgagctgccggtgctcgaaaccgtcacgcgggctgcacgttcgatgctcggactggccgcgtcggagttgcggccggacgcgcatttcaccgatctcggcggtgattcactgtccgcgctgtcgttttcgaccctgctgcaggacatgctcgaggtcgaggtcccggtcggtgtcatcgtgagccccgccaactcgctcgccgatctggcgaaatacatcgaggccgaacggcattcgggggtgcggcggccgagcctgatctcggtgcacggtcccggcaccgagatccgtgccgccgatctcaccctggacaagttcatcgacgagcgcaccctcgctgccgcgaaagcggttccggccgcgccggcccaggcgcagaccgtcctgctcaccggggcgaacggctatctcggccgcttcctgtgcctggaatggctgcagcgactggaccagaccggcggcacgctggtctgcatcgtgcgcggtaccgacgcggccgccgcgcggaagcgcctggatgcggtgttcgacagcggtgatccggagctgctcgaccactaccggaagctggccgccgagcacctcgaggtgctcgcgggcgatatcggcgacccgaatctcggcctggacgaagcgacttggcagcggctcgccgcgaccgtcgacctgatcgtgcaccccgccgccctcgtcaaccatgtgctgccgtacagccagctgttcgggccgaatgtggtcggcaccgccgagatcatccggctggccatcaccgagcgccgtaagcccgtgacgtacctgtcgacggtcgcggtggccgcacaggtcgatcccgccggcttcgacgaggagcgcgatatccgggagatgagcgcggtgcgctccatcgacgccgggtacgcgaacggttacggcaacagcaagtgggccggcgaggtgctgctgcgcgaggcccatgatctgtgcgggctgccggtcgccgtgttccgctcggacatgatcctggcgcacagcaaatacgtcggtcagctcaacgtccccgatgtgttcacccggctcatcctgagcctggcgctcaccggcatcgcaccgtattcgttctacgggacggacagcgccgggcagcgcaggcgggcccactacgacggtctgcccgccgatttcgtcgccgaggcgatcaccaccctcggcgcgcgagccgagtcggggttccatacctacgacgtgtggaacccgtacgacgacggcatctcgctggacgaattcgtcgactggctcggcgatttcggcgtgccgatccagcggatcgacgactacgacgaatggttccggcgtttcgagaccgcgatccgcgcgctgcccgaaaagcagcgcgatgcttcgctgctaccgctgctggacgcacaccggcggccactgcgcgcggtgcgcggttcgctgttgcccgccaagaacttccaggcggcggtgcagtccgcgcggatcggccccgatcaggacatcccgcatctttccccgcagttgatcgacaagtacgtcaccgacctgcgccacctcggcctgctctga

SEQ ID NO: 20 - protein, 1166 aa, Nocardia brasiliensis
MATDSRSDRLRRRIAQLFAEDEQVKAAVPDQEVVEAIRAPGLRLAQIMATVMERYADRPAVGQRASEPVTESGRTTFRLLPEFETLTYRELWARVRAVAAAWHGDAERPLRAGDFVALLGFAGIDYGTLDLANIHLGLVTVPLQSGATAPQLAAILAETTPRVLAATPDHLDIAVELLTGGASPERLVVFDYRPADDDHRAALESARRRLSDAGSAVVVETLDAVRARGSELPAAPLFVPAADEDPLALLIYTSGSTGTPKGAMYTERLNRTTWLSGAKGVGLTLGYMPMSHIAGRASFAGVLARGGTVYFTARSDMSTLFEDLALVRPTEMFFVPRVCDMIFQRYQAELSRRAPAAAASPELEQELKTELRLSAVGDRLLGAIAGSAPLSAEMREFMESLLDLELHDGYGSTEAGIGVLQDNIVQRPPVIDYKLVDVPELGYFRTDQPHPRGELLLKTEGMIPGYFRRPEVTAEIFDEDGFYRTGDIVAELEPDRLIYLDRRNNVLKLAQGEFVTVAHLEAVFATSPLIRQIYIYGNSERSFLLAVIVPTADALADGVTDALNTALTESLRQLAKEAGLQSYELPREFLVETEPFTVENGLLSGIAKLLRPKLKEHYGERLEQLYRDIEANRNDELIELRRTAAELPVLETVTRAARSMLGLAASELRPDAHFTDLGGDSLSALSFSTLLQDMLEVEVPVGVIVSPANSLADLAKYIEAERHSGVRRPSLISVHGPGTEIRAADLTLDKFIDERTLAAAKAVPAAPAQAQTVLLTGANGYLGRFLCLEWLQRLDQTGGTLVCIVRGTDAAAARKRLDAVFDSGDPELLDHYRKLAAEHLEVLAGDIGDPNLGLDEATWQRLAATVDLIVHPAALVNHVLPYSQLFGPNVVGTAEIIRLAITERRKPVTYLSTVAVAAQVDPAGFDEERDIREMSAVRSIDAGYANGYGNSKWAGEVLLREAHDLCGLPVAVFRSDMILAHSKYVGQLNVPDVFTRLILSLALTGIAPYSFYGTDSAGQRRRAHYDGLPADFVAEAITTLGARAESGFHTYDVWNPYDDGISLDEFVDWLGDFGVPIQRIDDYDEWFRRFETAIRALPEKQRDASLLPLLDAHRRPLRAVRGSLLPAKNFQAAVQSARIGPDQDIPHLSPQLIDKYVTDLRHLGLL

SEQ ID NO: 21 - DNA, 621 bp, Escherichia coli
atgaaaactacgcatacctccctcccctttgccggacatacgctgcattttgttgagttcgatccggcgaatttttgtgagcaggatttactctggctgccgcactacgcacaactgcaacacgctggacgtaaacgtaaaacagagcatttagccggacggatcgctgctgtttatgctttgcgggaatatggctataaatgtgtgcccgcaatcggcgagctacgccaacctgtctggcctgcggaggtatacggcagtattagccactgtgggactacggcattagccgtggtatctcgtcaaccgattggcattgatatagaagaaattttttctgtacaaaccgcaagagaattgacagacaacattattacaccagcggaacacgagcgactcgcagactgcggtttagccttttctctggcgctgacactggcattttccgccaaagagagcgcatttaaggcaagtgagatccaaactgatgcaggttttctggactatcagataattagctggaataaacagcaggtcatcattcatcgtgagaatgagatgtttgctgtgcactggcagataaaagaaaagatagtcataacgctgtgccaacacgattaa

SEQ ID NO: 22 - protein, 206 aa, Escherichia coli
MKTTHTSLPFAGHTLHFVEFDPANFCEQDLLWLPHYAQLQHAGRKRKTEHLAGRIAAVYALREYGYKCVPAIGELRQPVWPAEVYGSISHCGTTALAVVSRQPIGIDIEEIFSVQTARELTDNIITPAEHERLADCGLAFSLALTLAFSAKESAFKASEIQTDAGFLDYQIISWNKQQVIIHRENEMFAVHWQIKEKIVITLCQHD

SEQ ID NO: 23 - DNA, 654 bp, Corynebacterium glutamicum
atgctggatgagtctttgtttccaaattcggcaaagttttctttcattaaaactggcgatgctgttaatttagaccatttccatcagttgcatccgttggaaaaggcactggtagcgcactcggttgatattagaaaagcagagtttggagatgccaggtggtgtgcacatcaggcactccaagctttgggacgagatagcggtgatcccattttgcgtggggaacgaggaatgccattgtggccttcttcggtgtctggttcattgacccacactgacggattccgagctgctgttgtggcgccacgattgttggtgcgttctatgggattggatgccgaacctgcggagccgttgcccaaggatgttttgggttcaatcgctcgggtgggggagattcctcaacttaagcgcttggaggaacaaggtgtgcactgcgcggatcgcctgctgttttgtgccaaggaagcaacatacaaagcgtggttcccgctgacgcataggtggcttggttttgaacaagctgagatcgacttgcgtgatgatggcacttttgtgtcctatttgctggttcgaccaactccagtgccgtttatttcaggtaaatgggtactgcgtgatggttatgtcatagctgcgactgcagtgacttga

SEQ ID NO: 24 - protein, 217 aa, Corynebacterium glutamicum
MLDESLFPNSAKFSFIKTGDAVNLDHFHQLHPLEKALVAHSVDIRKAEFGDARWCAHQALQALGRDSGDPILRGERGMPLWPSSVSGSLTHTDGFRAAVVAPRLLVRSMGLDAEPAEPLPKDVLGSIARVGEIPQLKRLEEQGVHCADRLLFCAKEATYKAWFPLTHRWLGFEQAEIDLRDDGTFVSYLLVRPTPVPFISGKWVLRDGYVIAATAVT

SEQ ID NO: 25 - DNA, 1428 bp, Corynebacterium glutamicum
atgcgcctgcgtgtctcgagtagtctcctccccttcctcgtccccaacctcgaccattacggtcgccctctcctaaaggagcctggcatggatatccgccaaacaattaacgacacagcaatgtcgagatatcagtggttcattgtatttatcgcagtgctgctcaacgcactggacggctttgatgtcctcgccatgtcttttactgcgaatgcagtgaccgaagaatttggactgagtggcagccagcttggtgtgctgctgagttccgcgctgttcggcatgaccgctggatctttgctgttcggtccgatcggtgaccgtttcggccgtaagaatgccctgatgatcgcgctgctgttcaacgtggtgggattggtattgtccgccaccgcgcagtccgcaggccagttgggcgtgtggcgtttgatcactggtatcggcatcggcggaatcctcgcctgcatcacagtggtgatcagtgagttctccaacaacaaaaaccgcggcatggccatgtccatctacgctgctggttacggcatcggcgcgtccttgggcggattcggcgcagcgcagctcatcccaacatttggatggcgctccgtgttcgcagccggtgcgatcgcaactggtatcgccaccatcgctactttcttcttcctgccagaatccgttgattggctgagcactcgccgccctgcgggcgctcgcgacaagatcaattacattgcgcgccgcctgggcaaagtcggtacctttgagcttccaggcgaacaaagcttgtcgacgaaaaaagccggtctccaatcgtatgcagtgctcgttaacaaagagaaccgtggaaccagcatcaagctgtgggttgcgttcggcatcgtgatgttcggcttctacttcgccaacacttggaccccgaagctgctcgtggaaaccggaatgtcagaacagcagggcatcatcggtggtttgatgttgtccatgggtggagcattcggttccctgctctacggtttcctcaccaccaagttcagctcccgaaacacactgatgaccttcatggtgctgtccggcctgacgctgatcctgttcatttcctccacctctgttccatccatcgcgtttgccagcggcgttgtcgtgggcatgctgatcaatggttgtgtggctggtctgtacaccctgtccccacagctgtactccgctgaagtacgcaccactggtgtgggcgctgcgattggtatgggtcgtgtcggtgcgatttccgcgccactgctggtgggtagcctgctggattctggctggtccccaacgcagctgtatgttggtgtggcagtgattgttattgccggtgcaaccgcattgattgggatgcgcactcaggcagtagccgtcgaaaagcagcctgaagccctagcgaccaaatag

SEQ ID NO: 26 - protein, 475 aa, Corynebacterium glutamicum
MRLRVSSSLLPFLVPNLDHYGRPLLKEPGMDIRQTINDTAMSRYQWFIVFIAVLLNALDGFDVLAMSFTANAVTEEFGLSGSQLGVLLSSALFGMTAGSLLFGPIGDRFGRKNALMIALLFNVVGLVLSATAQSAGQLGVWRLITGIGIGGILACITVVISEFSNNKNRGMAMSIYAAGYGIGASLGGFGAAQLIPTFGWRSVFAAGAIATGIATIATFFFLPESVDWLSTRRPAGARDKINYIARRLGKVGTFELPGEQSLSTKKAGLQSYAVLVNKENRGTSIKLWVAFGIVMFGFYFANTWTPKLLVETGMSEQQGIIGGLMLSMGGAFGSLLYGFLTTKFSSRNTLMTFMVLSGLTLILFISSTSVPSIAFASGVVVGMLINGCVAGLYTLSPQLYSAEVRTTGVGAAIGMGRVGAISAPLLVGSLLDSGWSPTQLYVGVAVIVIAGATALIGMRTQAVAVEKQPEALATK

SEQ ID NO: 27 - DNA, 1296 bp, Corynebacterium glutamicum
gtgtcaacgaccaccccaacccgcgcaaccaaaagtgtcggaacagttctcgcactcctgtggttcgcaattgtcctcgacggctttgacctagtcgtcctgggcgcaacaatcccgtccatgctggaggatcccgcgtgggatctcactgctggacaggccacacagatttccaccatcggcctcgtcggcatgaccatcggcgcactgaccattggtttcttaactgaccgtctgggtcgacgccgcgtcatgctgttctctgtggcagtgttttctgtattcaccctcctgctggcattcaccaccaacgtccagctcttcagcctgtggcgtttcctcgcaggtgttggccttggtggagcactccccaccgcaattgccatggtgaccgagtttcgccccggcaccaaagcgggctctgcatcaactaccttgatgaccggataccacgtcggggcagtagcaaccgctttccttggtctcttccttatcgacggctttggttggcactccatgttcatcgcaggcgctgtgccaggactactcctgctgccactgctgtatttcttccttccagaatccccgcagtacctcaaaatctccggcaagttggatgaggcgcaggcagttgcagcatcttatggactttccctggatgatgatcttgatcgcgaacacgaagaagaacttggcgagtcctcctcactttcctccctgttcaagccctcgttccgccgcaacaccctggcgatttggggcacctcattcatgggactcctcctggtctacggcctgaacacatggctgccacaaatcatgcgccaagcagactacgacatgggtaactccctgggcttcctcatggttcttaacatcggcgcagtgatcggcctttatattgcagggcgaattgccgataagaactcccctcgcaaaacagcactcgtatggttcgtgttctctgcatttttcctcgcactacttgctgtccggatgccactgatcggtctgtatggcatcgtgctgctcaccggcatctttgtgttcagctcccaggtactcatctacgccttcgttggtgagaatcaccctgccaagatgcgtgcaactgccatgggattctccgcaggaattggtcgcctcggcgcgatctcgggtccgttgctgggcggcctgcttgtcagtgccaaccttgcttacccatggggcttcttcgccttcgctggcgttggactgctgggcgcgctgattttctccgcatcgaagactctgaggcatcgcgagaacgcttag

SEQ ID NO: 28 - protein, 431 aa, Corynebacterium glutamicum
MSTTTPTRATKSVGTVLALLWFAIVLDGFDLVVLGATIPSMLEDPAWDLTAGQATQISTIGLVGMTIGALTIGFLTDRLGRRRVMLFSVAVFSVFTLLLAFTTNVQLFSLWRFLAGVGLGGALPTAIAMVTEFRPGTKAGSASTTLMTGYHVGAVATAFLGLFLIDGFGWHSMFIAGAVPGLLLLPLLYFFLPESPQYLKISGKLDEAQAVAASYGLSLDDDLDREHEEELGESSSLSSLFKPSFRRNTLAIWGTSFMGLLLVYGLNTWLPQIMRQADYDMGNSLGFLMVLNIGAVIGLYIAGRIADKNSPRKTALVWFVFSAFFLALLAVRMPLIGLYGIVLLTGIFVFSSQVLIYAFVGENHPAKMRATAMGFSAGIGRLGAISGPLLGGLLVSANLAYPWGFFAFAGVGLLGALIFSASKTLRHRENA

SEQ ID NO: 29 - DNA, 1131 bp, Corynebacterium glutamicum
atgacactgtccgaacgcaagctcaccaccaccgccaagattcttccccacccactcaacgcctggtacgtcgccgcttgggattatgaagtcacatctaaaaagcccatggccaggacaatcgccaacaaaccactcgctttgtaccgcaccaaagatggccgagccgttgcccttgcagacgcctgctggcaccgcctcgcaccgctatccaagggaaaactcgtgggcacagacggaatccaatgcccttatcacggcttggagtacaactccgcgggccgctgcatgaaaatgcccgcgcaggaaaccctcaacccgtcagcagccgtcaactcctaccccgtggtggaagcccaccgctttgtgtgggtgtggctgggcgatcccacattggcagatcccacccaagtacccgatatgcaccagatgagccaccccgaatgggcaggcgatggacgcaccatctccgctgactgcaactaccaattagtgctggacaacttgatggacctcacccacgaagaattcgtgcactcctccagcatcggccaagacgaacttagtgaatcagagttcgtggtcacccacactgaagattccgtgacggtcacccgctggatgcatgacatagatgcaccaccgttttggcaaaagaacatgaatgataagttcccaggatttgaaggcaaggtggatcgttggcagatcatccactactactacccttccaccatctgcattgatgttggtgtagcaaaggctggaaccggcgcgcaggaaggcgaccgcagccagggcgttaatgggtatgtaatgaacaccattaccccagattcagatcgttcctctcattacttctgggcattcatgcgcaactaccgcctggaaagccaaaccatcaccacccagctgcgcgacggtgtatccggtgtattcaaagaagacgaagacatgctgaccgctcagcaagatgccatcgacgccaacaccgactatgagttttacagcctcaacattgatgccggtggcatgtgggtgcgccgaatcctcgaggaagcactctccaaggaaggccgactggatatccccaccacattcccccgcgcaacaccgaagccggaggcataa

SEQ ID NO: 30 - protein, 376 aa, Corynebacterium glutamicum
MTLSERKLTTTAKILPHPLNAWYVAAWDYEVTSKKPMARTIANKPLALYRTKDGRAVALADACWHRLAPLSKGKLVGTDGIQCPYHGLEYNSAGRCMKMPAQETLNPSAAVNSYPVVEAHRFVWVWLGDPTLADPTQVPDMHQMSHPEWAGDGRTISADCNYQLVLDNLMDLTHEEFVHSSSIGQDELSESEFVVTHTEDSVTVTRWMHDIDAPPFWQKNMNDKFPGFEGKVDRWQIIHYYYPSTICIDVGVAKAGTGAQEGDRSQGVNGYVMNTITPDSDRSSHYFWAFMRNYRLESQTITTQLRDGVSGVFKEDEDMLTAQQDAIDANTDYEFYSLNIDAGGMWVRRILEEALSKEGRLDIPTTFPRATPKPEA

SEQ ID NO: 31 - DNA, 978 bp, Corynebacterium glutamicum
atgaactcgcaatggcaagatgcacatgttgtttccagcgaaatcatcgctgcagacattcgacgaatagaactatccccgaaatttgcgattccagtaaaacccggcgaacatctcaagatcatggtgcccctaaaaactggacaggaaaagagatcgtactccatcgttgacgctcgtcacgacggttcgactctcgccctgagcgtactcaaaaccagaaactcccgtggaggatctgagttcatgcatacgcttcgagctggagacacagttactgtctccaggccgtctcaggattttcctctccgcgtgggtgcgcctgagtatgtacttgttgccggcggaattggaatcacagcgatccgttcaatggcatctttattaaagaaattgggagcaaactaccgcattcatttcgcagcacgcagccttgatgccatggcttacaaagatgagctcgtggcagaacacggcgacaagctgcacctgcatctagattctgaaggcaccaccatcgatgtcccagcattgatcgaaaccttaaacccccacactgagctttatatgtgcggccccatccgcttgatggatgccatccggcgcgcatggaacacccgcggacttgaccccaccaatctgcgtttcgaaacgtttggaaacagtggatggttctccccagaggttttccacatccaagtaccagagctggggcttcacgccacagtcaacaaggatgaaagcatgctggaggctttgcaaaaggctggggcgaatatgatgtttgattgtcgaaaaggcgaatgtggtttgtgccaggttcgcgttctagaagtcgatggccaggttgatcaccgcgatgtgttcttctctgatcgtcaaaaagaatccgacgcaaaggcatgcgcctgcgtgtctcgagtagtctcctccccttcctcgtccccaacctcgaccattacggtcgccctctcctaa

SEQ ID NO: 32 - protein, 325 aa, Corynebacterium glutamicum
MNSQWQDAHVVSSEIIAADIRRIELSPKFAIPVKPGEHLKIMVPLKTGQEKRSYSIVDARHDGSTLALSVLKTRNSRGGSEFMHTLRAGDTVTVSRPSQDFPLRVGAPEYVLVAGGIGITAIRSMASLLKKLGANYRIHFAARSLDAMAYKDELVAEHGDKLHLHLDSEGTTIDVPALIETLNPHTELYMCGPIRLMDAIRRAWNTRGLDPTNLRFETFGNSGWFSPEVFHIQVPELGLHATVNKDESMLEALQKAGANMMFDCRKGECGLCQVRVLEVDGQVDHRDVFFSDRQKESDAKACACVSRVVSSPSSSPTSTITVALS

SEQ ID NO: 33 - DNA, 615 bp, Corynebacterium glutamicum
atgattgatacagggaagaacggcgagttccgctacgagcagtcgaatatcatcgatcagaacgaagccgagttcggcatcactccttcacagaccgtgggcccttacgtccacatcggtttgacccttgaaggtgcggagcatctcgtggagccaggttcggaaggcgcggtgtcctttactgtttccgcaactgatggcaacggcgaccccatcgcggatgccatgtttgaactgtggcaggccgatccagagggcatccacaactctgatttggatccaaaccgcacagcaccagcaaccgcagatggcttccgcgggcttggtcgcgcgatggcaaacgcgcagggtgaggcaacgttcaccactttggttccgggagcattcgcagatgaggcaccacacttcaaggttggtgtgttcgcccgtggcatgctggagcgtctgtacactcgcgcatacctgccagacgccgatttgagcaccgacccagttttggctgtggtcccagctgatcgacgtgacctcctggtggctcaaaagaccgatgatggattccgcttcgacatcactgtccaggctgaagacaatgaaaccccattttttggactctaa

SEQ ID NO: 34 - protein, 204 aa, Corynebacterium glutamicum
MIDTGKNGEFRYEQSNIIDQNEAEFGITPSQTVGPYVHIGLTLEGAEHLVEPGSEGAVSFTVSATDGNGDPIADAMFELWQADPEGIHNSDLDPNRTAPATADGFRGLGRAMANAQGEATFTTLVPGAFADEAPHFKVGVFARGMLERLYTRAYLPDADLSTDPVLAVVPADRRDLLVAQKTDDGFRFDITVQAEDNETPFFGL

SEQ ID NO: 35 - DNA, 693 bp, Corynebacterium glutamicum
atggacatcccacacttcgccccgacgggaggcgaatactccccactgcacttcccggagtaccggaccaccatcaagcgcaacccaagcaacgatctcatcatggttcctagtcgcctcggcgagtccacgggacctgtcttcggcgaccgcgacttgggagacatcgacaacgacatgaccaaggtgaacggtggcgaggctatcggccagcgcatcttcgttcacggccgtgtcctcggtttcgatggcaagccagttccgcacaccttggtcgaggcgtggcaggcaaacgccgcaggccgttaccgccacaagaatgactcctggccagcgccactggatccacacttcaacggtgttgcacgtactctcaccgacaaggacggccagtaccacttctggaccgttatgccaggtaattacccttggggtaaccaccacaacgcatggcgcccggcgcacattcacttctcgctctatggtcgtcagtttacggagcgtctggtcacccagatgtacttcccgaacgatccattgttcttccaggatccgatctacaacgcggtgccaaagggtgcacgtgagcgcatgatcgcaacgttcgactatgacgagacccgtgaaaacttcgcgcttggttacaagttcgacatcgtccttcgtggccgcaacgccaccccatttgagtaa

SEQ ID NO: 36 - protein, 230 aa, Corynebacterium glutamicum
MDIPHFAPTGGEYSPLHFPEYRTTIKRNPSNDLIMVPSRLGESTGPVFGDRDLGDIDNDMTKVNGGEAIGQRIFVHGRVLGFDGKPVPHTLVEAWQANAAGRYRHKNDSWPAPLDPHFNGVARTLTDKDGQYHFWTVMPGNYPWGNHHNAWRPAHIHFSLYGRQFTERLVTQMYFPNDPLFFQDPIYNAVPKGARERMIATFDYDETRENFALGYKFDIVLRGRNATPFE

SEQ ID NO: 37 - DNA, 1164 bp, Escherichia coli
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 38 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 39 - DNA, 1062 bp, Corynebacterium glutamicum
atgagcatccaagtaaaagcactccagaaaaccggccccgaagcacctttcgaggtcaaaatcattgagcgtcgtgagcctcgcgctgacgacgtagttatcgacatcaaagctgccggcatctgccacagcgatatccacaccatccgcaacgaatggggcgaggcacacttcccgctcaccgtcggccacgaaatcgcaggcgttgtctctgcggttggctccgatgtaaccaagtggaaagtcggcgaccgcgttggcgtcggctgcctagttaactcctgcggcgaatgtgaacagtgtgtcgcgggatttgaaaacaactgccttcgcggaaacgtcggaacctacaactccgacgacgtcgacggcaccatcacgcaaggtggctacgccgaaaaggtagtggtcaacgaacgtttcctctgcagcatcccagaggaactcgacttcgatgtcgcagcaccactgctgtgcgcaggcatcaccacctactccccgatcgctcgctggaacgttaaagaaggcgacaaagtagcagtcatgggcctcggcgggctcggccacatgggtgtccaaatcgccgcagccaagggcgctgacgttaccgttctgtcccgttccctgcgcaaggctgaacttgccaaggaactcggcgcagctcgcacgcttgcgacttctgatgaggatttcttcaccgaacacgccggtgaattcgacttcatcctcaacaccattagcgcatccatcccagtcgacaagtacctgagccttctcaagccacacggtgtcatggctgttgtcggtctgccaccagagaagcagccactgagcttcggtgcgctcatcggcggcggaaaagtcctcaccggatccaacattggcggcatccctgaaacccaggaaatgctcgacttctgtgcaaaacacggcctcggcgcgatgatcgaaactgtcggcgtcaacgatgttgatgcagcctacgaccgcgttgttgccggcgacgttcagttccgcgttgtcattgatactgcttcgtttgcagaggtagaggcggtttag

SEQ ID NO: 40 - protein, 353 aa, Corynebacterium glutamicum
MSIQVKALQKTGPEAPFEVKIIERREPRADDVVIDIKAAGICHSDIHTIRNEWGEAHFPLTVGHEIAGVVSAVGSDVTKWKVGDRVGVGCLVNSCGECEQCVAGFENNCLRGNVGTYNSDDVDGTITQGGYAEKVVVNERFLCSIPEELDFDVAAPLLCAGITTYSPIARWNVKEGDKVAVMGLGGLGHMGVQIAAAKGADVTVLSRSLRKAELAKELGAARTLATSDEDFFTEHAGEFDFILNTISASIPVDKYLSLLKPHGVMAVVGLPPEKQPLSFGALIGGGKVLTGSNIGGIPETQEMLDFCAKHGLGAMIETVGVNDVDAAYDRVVAGDVQFRVVIDTASFAEVEAV

SEQ ID NO: 41 - DNA, 1113 bp, Corynebacterium glutamicum
gtgtccatgagcactgtcgtgcctggaattgtcgccctgtccaagggggcaccggtagaaaaagtaaacgttgttgtccctgatccaggtgctaacgatgtcatcgtcaagattcaggcctgcggtgtgtgccacaccgacttggcctaccgcgatggcgatatttcagatgagttcccttacctcctcggccacgaggcagcaggtattgttgaggaggtaggcgagtccgtcacccacgttgaggtcggcgatttcgtcatcttgaactggcgtgcagtgtgcggcgagtgccgtgcatgtaagaagggcgagccaaagtactgctttaacacccacaacgcatctaagaagatgaccctggaagacggcaccgagctgtccccagcactgggtattggcgcgttcttggaaaagaccctggtccacgaaggccagtgcaccaaggttaaccctgaggaagatccagcagcagctggccttctgggttgcggcatcatggcaggtcttggtgctgcggtaaacaccggtgatattaagcgcggcgagtccgtggcagtcttcggccttggtggcgtgggcatggcagctattgctggcgccaagattgctggtgcatcgaagattattgctgttgatatcgatgagaagaagttggagtgggcgaaggaattcggcgcaacccacaccattaattcctctggtcttggtggcgagggtgatgcctctgaggtcgtggcaaaggttcgtgagctcactgatggtttcggtactgacgtctccatcgatgcggtaggcatcatgccgacctggcagcaggcgttttactcccgtgatcatgcaggccgcatggtgatggtgggcgttccaaacctgacgtctcgcgtagatgttcctgcgattgatttttacggtcgcggtggctctgtgcgccctgcatggtacggcgactgcctgcctgagcgtgatttcccaacttatgtggatctgcacctgcagggtcgtttcccgctggataagtttgtttctgagcgtattggtcttgatgatgttgaagaggctttcaacaccatgaaggctggcgacgtgctgcgttctgtggtggagatctaa

SEQ ID NO: 42 - protein, 370 aa, Corynebacterium glutamicum
MSMSTVVPGIVALSKGAPVEKVNVVVPDPGANDVIVKIQACGVCHTDLAYRDGDISDEFPYLLGHEAAGIVEEVGESVTHVEVGDFVILNWRAVCGECRACKKGEPKYCFNTHNASKKMTLEDGTELSPALGIGAFLEKTLVHEGQCTKVNPEEDPAAAGLLGCGIMAGLGAAVNTGDIKRGESVAVFGLGGVGMAAIAGAKIAGASKIIAVDIDEKKLEWAKEFGATHTINSSGLGGEGDASEVVAKVRELTDGFGTDVSIDAVGIMPTWQQAFYSRDHAGRMVMVGVPNLTSRVDVPAIDFYGRGGSVRPAWYGDCLPERDFPTYVDLHLQGRFPLDKFVSERIGLDDVEEAFNTMKAGDVLRSVVEI

SEQ ID NO: 43 - DNA, 1047 bp, Corynebacterium glutamicum
gtgagttttatgaccactgctgcaccccaagaatttaccgctgctgttgttgaaaaattcggtcatgacgtgaccgtgaaggatattgaccttccaaagccagggccacaccaggcattggtgaaggtactcacctccggcatctgccacaccgacctccacgccttggagggcgattggccagtaaagccggaaccaccattcgtaccaggacacgaaggtgtaggtgaagttgttgagctcggaccaggtgaacacgatgtgaaggtcggcgatattgtcggcaatgcgtggctctggtcagcgtgcggcacctgcgaatactgcatcacaggcagggaaactcagtgtaacgaagctgagtacggtggctacacccaaaatggatccttcggccagtacatgctggtggatacccgatacgccgctcgcatcccagacggcgtggactacctcgaagcagcgccaattctgtgtgcaggcgtgactgtctacaaggcactcaaagtctctgaaacccgcccgggccaattcatggtgatctccggtgtcggcggacttggccacatcgcagtccaatacgcagcggcgatgggcatgcgtgtcattgcggtagatattgccgaggacaagctggaacttgcccgtaagcacggtgcggaatttaccgtgaatgcgcgtaatgaagatccaggcgaagctgtacagaagtacaccaacggtggcgcacacggcgtgcttgtgactgcagttcacgaggcagcattcggccaggcactggatatggctcgacgtgcaggaacaattgtgttcaacggtctgccaccgggagagttcccagcatccgtgttcaacatcgtattcaagggcctgaccatccgtggatccctcgtgggaacccgccaagacttggccgaagcgctcgatttctttgcacgcggactaatcaagccaaccgtgagtgagtgctccctcgatgaggtcaatggagttcttgaccgcatgcgaaacggcaagatcgatggtcgtgtggcgattcgtttctaa

SEQ ID NO: 44 - protein, 348 aa, Corynebacterium glutamicum
MSFMTTAAPQEFTAAVVEKFGHDVTVKDIDLPKPGPHQALVKVLTSGICHTDLHALEGDWPVKPEPPFVPGHEGVGEVVELGPGEHDVKVGDIVGNAWLWSACGTCEYCITGRETQCNEAEYGGYTQNGSFGQYMLVDTRYAARIPDGVDYLEAAPILCAGVTVYKALKVSETRPGQFMVISGVGGLGHIAVQYAAAMGMRVIAVDIAEDKLELARKHGAEFTVNARNEDPGEAVQKYTNGGAHGVLVTAVHEAAFGQALDMARRAGTIVFNGLPPGEFPASVFNIVFKGLTIRGSLVGTRQDLAEALDFFARGLIKPTVSECSLDEVNGVLDRMRNGKIDGRVAIRF

SEQ ID NO: 45 - DNA, 1020 bp, Corynebacterium glutamicum
atgcccaaatacattgccatgcaggtatccgaatccggtgcaccgttagccgcgaatctcgtgcaacctgctccgttgaaatcgagggaagtccgcgtggaaatcgctgctagtggtgtgtgccatgcagatattggcacggcagcagcatcggggaagcacactgtttttcctgttacccctggtcatgagattgcaggaaccatcgcggaaattggtgaaaacgtatctcggtggacggttggtgatcgcgttgcaatcggttggtttggtggcaattgcggtgactgcgctttttgtcgtgcaggtgatcctgtgcattgcagagagcggaagattcctggcgtttcttatgcgggtggttgggcacagaatattgttgttccagcggaggctcttgctgcgattccagatggcatggacttttacgaggccgccccgatgggctgcgcaggtgtgacaacattcaatgcgttgcgaaacctgaagctggatcccggtgcggctgtcgcggtctttggaatcggcggtttagtgcgcctagctattcagtttgctgcgaaaatgggttatcgaaccatcaccatcgcccgcggtttagagcgtgaggagctagctaggcaacttggcgccaaccactacatcgatagcaatgatctgcaccctggccaggcgttatttgaacttggcggggctgacttgatcttgtctactgcgtccaccacggagcctctttcggagttgtctaccggtctttctattggcgggcagctaaccattatcggagttgatgggggagatatcaccgtttcggcagcccaattgatgatgaaccgtcagatcatcacaggtcacctcactggaagtgcgaatgacacggaacagactatgaaatttgctcatctccatggcgtgaaaccgcttattgaacggatgcctctcgatcaagccaacgaggctattgcacgtatttcagctggtaaaccacgtttccgtattgtcttggagccgaattcataa

SEQ ID NO: 46 - protein, 339 aa, Corynebacterium glutamicum
MPKYIAMQVSESGAPLAANLVQPAPLKSREVRVEIAASGVCHADIGTAAASGKHTVFPVTPGHEIAGTIAEIGENVSRWTVGDRVAIGWFGGNCGDCAFCRAGDPVHCRERKIPGVSYAGGWAQNIVVPAEALAAIPDGMDFYEAAPMGCAGVTTFNALRNLKLDPGAAVAVFGIGGLVRLAIQFAAKMGYRTITIARGLEREELARQLGANHYIDSNDLHPGQALFELGGADLILSTASTTEPLSELSTGLSIGGQLTIIGVDGGDITVSAAQLMMNRQIITGHLTGSANDTEQTMKFAHLHGVKPLIERMPLDQANEAIARISAGKPRFRIVLEPNS

SEQ ID NO: 47 - DNA, 879 bp, Corynebacterium glutamicum
atgcaaacccttgctgctattgttcgtgccacgaagcaaccttttgagatcaccaccattgatctggatgcaccacgaccagatgaagttcaaatccgtgttattgctgccggagtgcgccacactgacgcaattgttcgtgatcagatttacccaacttttcttcccgcagttttcggccacgaaggcgccggagtagttgtcgccgtgggttctgcagtcacctcggtgaaaccagatgacaaggtagtgctgggattcaactcttgtggccagtgcttgaagtgtttgggcggtaagcctgcgtactgtgagaaattctatgaccgcaacttcgcatgcacccgcgatgccgggcacactactttgtttacccgtgcaacaaaagagcaggcagaggccatcatcgacacccttgatgatgttttctacgatgcggatgcgggtttcctggcatacccagcaactcccccagaggcttcgggagtaagcgtgttggttgtcgcggctggtacctctgatctcccccaagcaaaggaagcactacacactgcctcctacttggggcgctccacctcactgattgttgattttggagtggctggcatccaccgcctgctttcatacgaagaagaactccgcgctgcgggcgtgctcatcgttgccgctggaatggatggtgcgctacccggagttgtcgcaggcttagtgtccgcacctgtcgtcgcactgccaacctccgtgggatacggcgcaggtgctggaggaatcgcaccacttctgaccatgcttaacgcctgcgcgccgggagttggagtggtcaacattgataacggctatggagcaggacacctggctgcgcagattgcggcgaggtaa

SEQ ID NO: 48 - protein, 292 aa, Corynebacterium glutamicum
MQTLAAIVRATKQPFEITTIDLDAPRPDEVQIRVIAAGVRHTDAIVRDQIYPTFLPAVFGHEGAGVVVAVGSAVTSVKPDDKVVLGFNSCGQCLKCLGGKPAYCEKFYDRNFACTRDAGHTTLFTRATKEQAEAIIDTLDDVFYDADAGFLAYPATPPEASGVSVLVVAAGTSDLPQAKEALHTASYLGRSTSLIVDFGVAGIHRLLSYEEELRAAGVLIVAAGMDGALPGVVAGLVSAPVVALPTSVGYGAGAGGIAPLLTMLNACAPGVGVVNIDNGYGAGHLAAQIAAR

SEQ ID NO: 49 - DNA, 819 bp, Escherichia coli
atggaaacctatgctgtttttggtaatccgatagcccacagcaaatcgccattcattcatcagcaatttgctcagcaactgaatattgaacatccctatgggcgcgtgttggcacccatcaatgatttcatcaacacactgaacgctttctttagtgctggtggtaaaggtgcgaatgtgacggtgccttttaaagaagaggcttttgccagagcggatgagcttactgaacgggcagcgttggctggtgctgttaataccctcatgcggttagaagatggacgcctgctgggtgacaataccgatggtgtaggcttgttaagcgatctggaacgtctgtcttttatccgccctggtttacgtattctgcttatcggcgctggtggagcatctcgcggcgtactactgccactcctttccctggactgtgcggtgacaataactaatcggacggtatcccgcgcggaagagttggctaaattgtttgcgcacactggcagtattcaggcgttgagtatggacgaactggaaggtcatgagtttgatctcattattaatgcaacatccagtggcatcagtggtgatattccggcgatcccgtcatcgctcattcatccaggcatttattgctatgacatgttctatcagaaaggaaaaactccttttctggcatggtgtgagcagcgaggctcaaagcgtaatgctgatggtttaggaatgctggtggcacaggcggctcatgcctttcttctctggcacggtgttctgcctgacgtagaaccagttataaagcaattgcaggaggaattgtccgcgtga

SEQ ID NO: 50 - protein, 272 aa, Escherichia coli
METYAVFGNPIAHSKSPFIHQQFAQQLNIEHPYGRVLAPINDFINTLNAFFSAGGKGANVTVPFKEEAFARADELTERAALAGAVNTLMRLEDGRLLGDNTDGVGLLSDLERLSFIRPGLRILLIGAGGASRGVLLPLLSLDCAVTITNRTVSRAEELAKLFAHTGSIQALSMDELEGHEFDLIINATSSGISGDIPAIPSSLIHPGIYCYDMFYQKGKTPFLAWCEQRGSKRNADGLGMLVAQAAHAFLLWHGVLPDVEPVIKQLQEELSA

SEQ ID NO: 51 - DNA, 35 bp, Artificial Sequence
  primer
cggtacccggggatccttacttccgcgtatccaac

SEQ ID NO: 52 - DNA, 41 bp, Artificial Sequence
  primer
ctaggaatcgcggccggtgaactcctaaagaactatataac

SEQ ID NO: 53 - DNA, 20 bp, Artificial Sequence
  primer
ggccgcgattcctagcatgc

SEQ ID NO: 54 - DNA, 35 bp, Artificial Sequence
  primer
ccaagcttgcatgccagtcatcatcaacggtgccg

SEQ ID NO: 55 - DNA, 21 bp, Artificial Sequence
  primer
atctccgcagaagacgtactg

SEQ ID NO: 56 - DNA, 20 bp, Artificial Sequence
  primer
tccgatcatgtatgacctcc

SEQ ID NO: 57 - DNA, 36 bp, Artificial Sequence
  primer
cggtacccggggatcggcatagtgcttccaacgctc

SEQ ID NO: 58 - DNA, 36 bp, Artificial Sequence
  primer
tagctccactcaagattcctcgatattacctacagg

SEQ ID NO: 59 - DNA, 20 bp, Artificial Sequence
  primer
tcttgagtggagctagggcc

SEQ ID NO: 60 - DNA, 37 bp, Artificial Sequence
  primer
ccaagcttgcatgcccatatagagcccaggagctctc

SEQ ID NO: 61 - DNA, 23 bp, Artificial Sequence
  primer
cgccgcaaagtccaaatagaaag

SEQ ID NO: 62 - DNA, 20 bp, Artificial Sequence
  primer
ggattcttcctgaactcagc

SEQ ID NO: 63 - DNA, 36 bp, Artificial Sequence
  primer
cggtacccggggatcgggctcgtcctgaaattgcac

SEQ ID NO: 64 - DNA, 35 bp, Artificial Sequence
  primer
tccgtcgtgagccatgttgtgcccacgagactacc

SEQ ID NO: 65 - DNA, 20 bp, Artificial Sequence
  primer
atggctcacgacggattgcg

SEQ ID NO: 66 - DNA, 36 bp, Artificial Sequence
  primer
ccaagcttgcatgcccggttgcagccttcataaacg

SEQ ID NO: 67 - DNA, 21 bp, Artificial Sequence
  primer
agaccaatgagtacccaaccg

SEQ ID NO: 68 - DNA, 20 bp, Artificial Sequence
  primer
tcagcgtctggctcagctac

SEQ ID NO: 69 - DNA, 36 bp, Artificial Sequence
  primer
cggtacccggggatcaaccccagctcaaataacacc

SEQ ID NO: 70 - DNA, 37 bp, Artificial Sequence
  primer
tttcaacacaatccgtccttctcgcttggattacttg

SEQ ID NO: 71 - DNA, 23 bp, Artificial Sequence
  primer
cggattgtgttgaaattgctctg

SEQ ID NO: 72 - DNA, 35 bp, Artificial Sequence
  primer
ccaagcttgcatgcctcaccacgggaatcttcagg

SEQ ID NO: 73 - DNA, 20 bp, Artificial Sequence
  primer
ccggactggggtgtgttttg

SEQ ID NO: 74 - DNA, 20 bp, Artificial Sequence
  primer
cccggaaaatacggtatagc

SEQ ID NO: 75 - DNA, 35 bp, Artificial Sequence
  primer
ccaagcttgcatgccccatcgcattgccgaaaagc

SEQ ID NO: 76 - DNA, 35 bp, Artificial Sequence
  primer
aaagatcgggtcaatgcagttcgcggggcgaacat

SEQ ID NO: 77 - DNA, 35 bp, Artificial Sequence
  primer
cgccccgcgaactgcattgacccgatctttatacc

SEQ ID NO: 78 - DNA, 35 bp, Artificial Sequence
  primer
cggtacccggggatcaacgttgacggtgatgccat

SEQ ID NO: 79 - DNA, 25 bp, Artificial Sequence
  primer
gaaatgtcatacttcagccatcagg

SEQ ID NO: 80 - DNA, 25 bp, Artificial Sequence
  primer
tgcgagtgatgaaatcctgaaactt

SEQ ID NO: 81 - DNA, 2180 bp, Artificial Sequence
  promoter P2
tttcgcggtgaatcaacccacccgaacggcgatccttcgaagtattttgccgattgatcaattagcgtcggcaacatcactgaatatgctgctcatgcagaccggcgcaatcgttggcccgctgatcgcaggtgcgttgattccgctgatcggtttcgggtggctgtatttccttgatgttgtctccatcatccccacactgtgggctgtatggtcactgccttcaatcaagccatccggcaaggtcatgaaggccggtttcgccagtgtggtggatggcctgaagtatttggctggccaacccgtgttgttgatggtgatggtgctggatcttatcgccatgattttcggcatgccacgtgcgctttaccccgagatcgcggaagtgaacttcggtggtggtgacgccggtgcaacgatgctggcgttcatgtactcatccatggctgttggcgcagttcttggcggcgtgctgtctggttgggtttcccggattagccgccagggtgttgcagtttattggtgcatcatcgcctggggcgcagccgttgctttgggtggcgtagcaattgttgtcagccccggcgctgtgaccgcgtgggcgtggatgttcatcatcatgatggtcattggtggcatggctgacatgtttagctcggctgttcgaaatgctattttgcagcagtctgcagcggaacatgtgcagggccgaatccaaggtgtgtggatcatcgtcgtggtgggtggacctcgtttagctgacgtccttcacggttgggccgctgagcccttgggtgcaggttggacggtattatggggcggagtagcggtggttgtactcactgcaatttgtatggtggcggtgcctaaattctggaaatacgagaaaccaaaaattaccggcatctaaatacttatccatgcccatttacagacaatgccttagctttgacctgcacaaatagttgcaaattgtcccacatacacataaagtagcttgcgtatttaaaattatgaacctaaggggtttagcaatgcccaatcaggcccacttctctgcgtcctttgcccgcccctctaccccggctgcaaagtgcatgcaccatatccgcctcggccagcaactcattagaaatgagctggtcgaggccacaggtctgtcccaaccgactgtcacccgcgcagtcaccgctttaatgcaggcaggtttggttcgtgaacgccctgatctcacactctcatcgggccctggtcgtcccaatattcctctagaactcgctccaagtccatggattcatgcaggcgtggcaatcggcaccaagtcttcctacgtcgctttgtttgataccaagggtcgcacccttcgtgatgccatactggaaatctcagcagctgatttagatccagacaccttcatcgaacacctcatcgctggtgtcaaccgcctcaccactggtcttgatctaccactggtaggtattggtgtggctacctcaggaaaagtcaccaacgcgggcgttgtcaccgcaagcaacttgggctgggatggcgttgatatcgccggccgtctgaactaccaattcagcgttccagcaaccgtggcatcagcaattcctgccatcgcagcttctgaactgcaggcttccccacttccccaccctgagcagccaactcccatcaccttgaccttctacgccgatgactctgtgggcgcggcctacagcaatgatttgggagtacatgtcattggaccactggctacaactcgtggatcaggtttggatactttgggcatggctgctgaagatgcgctgagcacccaaggtttcttaagcagggtttctgatcagggtatctttgccaacagccttggtgagctagtcaccattgctaaagacaatgaaaccgcacgggaattcctcaacgatcgcgcgaccctgctggctcacactgccgcagaagctgctgaaacagttaagccatccaccctggttctctcgggatcggcgttttccgaagatccacaaggtcggttggtgttcgcttcccaattgaagaaggaatacgacgcagacattgagctccgcttgatccccacccaccgggaaaatgtccgcgcagcagctcgcgcagtcgcacttgatcgactactcaacgagccacttaccctcgtaccctaa

SEQ ID NO: 82 - DNA, 2247 bp, Artificial Sequence
  promoter P4
ccctcatgagttcaggggttagaaaagcaatgggatttggatgcggttcggttttggccgtcatcatggttatctcatttgttggatgggcgcttagcttcatggatggaacggcacctattcgccaactccagcaaatccctgaagatgttccgccggcgcgtggtgtagaagttccgcaaattgatacaggggcagatggacgcacgtcagatcatttgcgtttttgggcggaaccaattgctcaagatgctggtgtgtccgctcaagcgattgcggcttatggaaacgcagagctcattgcgagtactgcgtggcctggctgcaatctggggtggaataccttggcaggtatcggccaggtggaaacccgtcacggtacctacaacggcaaaatgttcgggggcagttccctggatgaaaatggagttgcaacccctccaatcatcggcgttccacttgatggttcaccggggtttgcggaaattcccgacactgatggtggggaattagatggcgatactgaatatgatcgcgcggtaggtcccatgcagttcattccggaaacgtggcgacttatgggattggatgcaaacggtgatggggtagcggaccccaaccaaattgatgacgcagcattgagtgccgcaaacctgttgtgttccaacgatcgtgacttgtccactcctgaaggatggaccgcagctgttcattcttacaacatgtctaatcagtatttgatggacgttcgagatgctgccgcgtcctacgctttacgacagccggcgatctaaaacttaacaagcgcaacccccgaaaatgtgagattatgtccggtcggacacgtgcgggctggggatatgggtagtttaataaatttataccacacagtctattgcaatagaccaagctgttcagtagggtgcatgggagaagaatttcctaataaaaactcttaaggacctccaagtggctgaaatcatgcacgtattcgctcgcgaaattctcgactcccgcggtaacccaaccgtcgaggcagaggttttccttgatgacggttcccacggtgtcgcaggtgttccatccggcgcatccaccggcgtccacgaggctcatgagctgcgtgacggtggcgatcgctacctgggcaagggcgttttgaaggcagttgaaaacgtcaacgaagaaatcggcgacgagctcgctggcctagaggctgacgatcagcgcctcatcgacgaagcaatgatcaagcttgatggcaccgccaacaagtcccgcctgggtgcaaacgcaatccttggtgtttccatggctgttgcaaaggctgctgctgattccgcaggcctcccactgttccgctacatcggtggaccaaacgcacacgttcttccagttccaatgatgaacatcatcaacggtggcgctcacgctgactccggtgttgacgttcaggaattcatgatcgctccaatcggtgcagagaccttctctgaggctctccgcaacggcgcagaggtctaccacgcactgaagtccgtcatcaaggaaaagggcctgtccaccggacttggcgatgagggcggcttcgctccttccgtcggctccacccgtgaggctcttgaccttatcgttgaggcaatcgagaaggctggcttcaccccaggcaaggacatcgctcttgctctggacgttgcttcctctgagttcttcaaggacggcacctaccacttcgaaggtggccagcactccgcagctgagatggcaaacgtttacgctgagctcgttgacgcgtacccaatcgtctccatcgaggacccactgcaggaagatgactgggagggttacaccaacctcaccgcaaccatcggcgacaaggttcagatcgttggcgacgacttcttcgtcaccaaccctgagcgcctgaaggagggcatcgctaagaaggctgccaactccatcctggttaaggtgaaccagatcggtaccctcaccgagaccttcgacgctgtcgacatggctcaccgcgcaggctacacctccatgatgtcccaccgttccggtgagaccgaggacaccaccattgctgacctcgcagttgcactcaactgtggccagatcaagactggtgctccagcacgttccgaccgtgtcgcaaagtacaaccagcttctccgcatcgagcagttgcttggcgacgccggcgtctacgcaggtcgcagcgcattcccacgctttcagggctaa

SEQ ID NO: 83 - DNA, 2192 bp, Artificial Sequence
  promoter P8
tcaccgagtctttgatcaagggtggcgcttttgactcccttggacacgcacgaaaaggcctcatgctggtcttcgaagatgccgttgattccgtcatcgctaccaaaaaagctgctgacaagggacaatttgatctctttgcagctttcgactcggataacaacgacgatgtggcaagtttcttccagatcaccgttcctgatgacgaatgggaccgtaagcatgagctcgcactcgagcgagaaatgctgggtctgtatgtttctggacacccactcgatggctatgaagatgccattgctgcccaggttgatacagcactgaccaccattgttgccggtgaactcaagcacggcgcagaagtgaccgtgggtggcattatctctggtgtggatcgacggttctccaagaaggacggttccccttgggcgattgtcaccattgaagatcacaacggcgcgtccgttgaattgttggtcttcaacaaggtgtattccatcgttggatccatgattgtggaagacaacatcattttggccaaggcacacatctccattcgagatgatcgtatgagccttttctgtgatgatctccgcgttccagagcttgggccaggaaacgggcaaggacttccgcttcgtttgtccatgcgtactgatcagtgcaccatgtccaacattgccaagctcaagcaggtgctggtggacaacaagggtgaatctgatgtgtacctcaatttgatcgatggggataactccacggtcatgattttgggtgatcacttaagagtcaaccgatccgcaagtttgatgggcgacctcaaggcaacgatggggccaggcatcctcggttaatcacatcacactgggattaccccgtgtaggggtgaaaacccgaatgtggctaaaacttttggaaacttaagttacctttaatcggaaacttattgaattcgggtgaggcaactgcaactctggacttaaagcatgagccagaaccgcatcaggaccactcacgttggttccttgccccgtaccccagagctacttgatgcaaacatcaagcgctctaacggtgagattggggaggaggaattcttccagatcctgcagtcttctgtagatgacgtgatcaagcgccaggttgacctgggtatcgacatcctcaacgagggcgaatacggccacgtcacctccggtgcagttgacttcggtgcatggtggaactactccttcacccgcctgggcggactgaccatgaccgataccgaccgttgggcaagccaggaagcagtgcgttccacccctggcaacatcaagctgaccagcttctctgatcgtcgcgaccgcgcattgttcagcgaagcatacgaggatccagtatctggcatcttcaccggccgcgcttctgtgggcaacccagagttcaccggacctattacctacattggccaggaagaaactcagacggatgttgatctgctgaagaagggcatgaacgcagcgggagctaccgacggcttcgttgcagcactatccccaggatctgcagctcgattgaccaacaagttctacgacactgatgaagaagtcgtcgcagcatgtgccgatgcgctttcccaggaatacaagatcatcaccgatgcaggtctgaccgttcagctcgacgcaccggacttggcagaagcatgggatcagatcaacccagagccaagcgtgaaggattacttagactggatcggtacacgcatcgatgccatcaacagtgcagtgaagggccttccaaaggaacagacccgcctgcacatctgctggggctcttggcacggaccacacgtcactgacatcccattcggtgacatcattggtgagatcctgcgcgcagaggtcggtggcttctccttcgaaggcgcatctcctcgtcacgcacacgagtggcgtgtatgggaagaaaacaagcttcctgaaggctctgttatctaccctggtgttgtgtctcactccatcaacgctgtggagcacccacgcctggttgctgatcgtatcgttcagttcgccaagcttgttggccctgagaacgtcattgcgtccactgactgtggtctgggcggacgtctgcattcccagatcgcatgggcaaagctggagtccctagtagagggcgctcgcattgcatcaaaggaactgttctaa

SEQ ID NO: 84 - DNA, 97 bp, Artificial Sequence
  promoter P3
tgccgtttctcgcgttgtgtgtggtactacgtggggacctaagcgtgtaagatggaaacgtctgtatcggataagtagcgaggagtgttcgttaaaa

SEQ ID NO: 85 - DNA, 35 bp, Artificial Sequence
  primer
ccaagcttgcatgcctcaccgagtctttgatcaag

SEQ ID NO: 86 - DNA, 35 bp, Artificial Sequence
  primer
caaaagttttagccacattcgggttttcaccccta

SEQ ID NO: 87 - DNA, 35 bp, Artificial Sequence
  primer
ctctggacttaaagcatgagccagaaccgcatcag

SEQ ID NO: 88 - DNA, 35 bp, Artificial Sequence
  primer
cggtacccggggatcttagaacagttcctttgatg

SEQ ID NO: 89 - DNA, 87 bp, Artificial Sequence
  DNA fragment of P8 promoter
gtggctaaaacttttggaaacttaagttacctttaatcggaaacttattgaattcgggtgaggcaactgcaactctggacttaaagc

SEQ ID NO: 90 - DNA, 35 bp, Artificial Sequence
  primer
gtgaaaacccgaatgtggctaaaacttttggaaac

SEQ ID NO: 91 - DNA, 35 bp, Artificial Sequence
  primer
gcggttctggctcatgctttaagtccagagttgca

SEQ ID NO: 92 - DNA, 1206 bp, Corynebacterium glutamicum
atgagccagaaccgcatcaggaccactcacgttggttccttgccccgtaccccagagctacttgatgcaaacatcaagcgttctaacggtgagattggggaggaggaattcttccagattctgcagtcttctgtagatgacgtgatcaagcgccaggttgacctgggtatcgacatccttaacgagggcgaatacggccacgtcacctccggtgcagttgacttcggtgcatggtggaactactccttcacccgcctgggcggactgaccatgaccgataccgaccgttgggcaagccaggaagcagtgcgttccacccctggcaacatcaagctgaccagcttctctgatcgtcgcgaccgcgcattgttcagcgaagcatacgaggatccagtatctggcatcttcaccggtcgcgcttctgtgggcaacccagagttcaccggacctattacctacattggccaggaagaaactcagacggatgttgatctgctgaagaagggcatgaacgcagcgggagctaccgacggcttcgttgcagcactatccccaggatctgcagctcgattgaccaacaagttctacgacactgatgaagaagtcgtcgcagcatgtgctgatgcgctttcccaggaatacaagatcatcaccgatgcaggtctgaccgttcagctcgacgcaccggacttggcagaagcatgggatcagatcaacccagagccaagcgtgaaggattacttggactggatcggtacacgcatcgatgccatcaacagtgcagtgaagggccttccaaaggaacagacccgcctgcacatctgctggggctcttggcacggaccacacgtcactgacatcccattcggtgacatcattggtgagatcctgcgcgcagaggtcggtggcttctccttcgaaggcgcatctcctcgtcacgcacacgagtggcgtgtatgggaagaaaacaagcttcctgaaggctctgttatctaccctggtgttgtgtctcactccatcaacgctgtggagcacccacgcctggttgctgatcgtatcgttcagttcgccaagcttgttggccctgagaacgtcattgcgtccactgactgtggtctgggcggacgtctgcattcccagatcgcatgggcaaagctggagtccctagtagagggcgctcgcattgcatcaaaggaactgttctaa

SEQ ID NO: 93 - protein, 401 aa, Corynebacterium glutamicum
MSQNRIRTTHVGSLPRTPELLDANIKRSNGEIGEEEFFQILQSSVDDVIKRQVDLGIDILNEGEYGHVTSGAVDFGAWWNYSFTRLGGLTMTDTDRWASQEAVRSTPGNIKLTSFSDRRDRALFSEAYEDPVSGIFTGRASVGNPEFTGPITYIGQEETQTDVDLLKKGMNAAGATDGFVAALSPGSAARLTNKFYDTDEEVVAACADALSQEYKIITDAGLTVQLDAPDLAEAWDQINPEPSVKDYLDWIGTRIDAINSAVKGLPKEQTRLHICWGSWHGPHVTDIPFGDIIGEILRAEVGGFSFEGASPRHAHEWRVWEENKLPEGSVIYPGVVSHSINAVEHPRLVADRIVQFAKLVGPENVIASTDCGLGGRLHSQIAWAKLESLVEGARIASKELF

SEQ ID NO: 94 - DNA, 666 bp, Homo sapiens
atgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccttaa

SEQ ID NO: 95 - DNA, 675 bp, Homo sapiens
catatgggggataccaaagaacagcggattctgaatcatgttttacagcacgctgaaccgggcaatgctcagagcgttttagaagcaatagatacctattgtgaacagaaggaatgggcaatgaatgttggcgataaaaagggcaaaattgttgatgcagttatccaggaacatcagccgagcgttttacttgaactgggcgcatattgcggttattccgcagttcggatggcacggctgctgtcccctggcgctcgcttaattaccattgaaattaatccggattgcgcagcaattacccagcggatggttgactttgcaggtgttaaagataaagtcaccttggttgtcggcgctagccaggatattattccgcagctgaaaaaaaaatacgatgttgataccctggatatggtctttttagatcattggaaagatcggtatctgcccgatactctgttattggaagagtgcggtctgctgcggaaaggcaccgttctactggcagataatgttatttgtcctggggctcctgattttctagctcatgttcggggcagcagctgtttcgaatgtacccattaccaatcgtttctggaatatcgcgaagttgttgatggtctggaaaaggcaatttataaaggtcctggtagcgaggctggcccgtaagagctc

SEQ ID NO: 96 - DNA, 6557 bp, Artificial Sequence
  pELAC vector
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctgcgggcagtgagcgcaacgcaattaatgtgagttagctcactcattaggcaccccaggctttacactttatgcttccggctcgtataatgtgtggaattgtgagcggataacaatttcacacaggatctagatttaagaaggagatatacatatggccgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaacaggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaaggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 97 - DNA, 22 bp, Artificial Sequence
  primer
gtgagttagctcactcattagg

SEQ ID NO: 98 - DNA, 22 bp, Artificial Sequence
  primer
cgattggtaatgggtacattcg

SEQ ID NO: 99 - DNA, 20 bp, Artificial Sequence
  primer
ggatctcagtggtggtggtg

SEQ ID NO: 100 - DNA, 44 bp, Artificial Sequence
  primer
ccaagcttgcatgccaaattcctgtgaattagctgatttagtac

SEQ ID NO: 101 - DNA, 38 bp, Artificial Sequence
  primer
tttggtatcccccatagaggcgaaggctccttgaatag

SEQ ID NO: 102 - DNA, 22 bp, Artificial Sequence
  primer
atgggggataccaaagaacagc

SEQ ID NO: 103 - DNA, 38 bp, Artificial Sequence
  primer
cggtacccggggatcatgctagttattgctcagcggtg

SEQ ID NO: 104 - DNA, 20 bp, Artificial Sequence
  primer
tgacatcccattcggtgaca

SEQ ID NO: 105 - DNA, 20 bp, Artificial Sequence
  primer
ttcttcccatacacgccact

SEQ ID NO: 106 - DNA, 20 bp, Artificial Sequence
  primer
aggtggggatgacgtcaaat

SEQ ID NO: 107 - DNA, 20 bp, Artificial Sequence
  primer
gaactgaggccggctttaag